(19) United States
(12) Patent Application Publication
(10) Pub. No.: US 2010/0120716 A1
(43) Pub. Date: May 13, 2010

(54) MACROCYCLIC HEPATITIS C SERINE PROTEASE INHIBITORS AND USES THEREFOR

(75) Inventors: David A. Campbell, San Diego, CA (US); Michael E. Hepperle, San Diego, CA (US); David T. Winn, San Diego, CA (US); Juan M. Betancort, San Diego, CA (US)

Correspondence Address: SCHWEGMAN, LUNDBERG & WOESSNER, P.A., P.O. BOX 2938, MINNEAPOLIS, MN, 55402, US

(73) Assignee: Phenomix Corporation, San Diego, CA (US)

(21) Appl. No.: 12/515,941

(22) PCT Filed: Dec. 5, 2007
(86) PCT No.: PCT/US07/86530

§ 371 Date: Jan. 8, 2010

Related U.S. Application Data
(60) Provisional application No. 60/873,146, filed on Dec. 6, 2006.
(60) Provisional application No. 60/917,314, filed on May 10, 2007.

(51) Int. Cl. A61K 31/69; C07D 267/00; C07D 245/04; A61P 31/12

(52) U.S. Cl. 514/64; 540/452

(57) ABSTRACT

Macrocyclic inhibitors of Hepatitis C protease are provided, the inhibitors including a boronic acid or ester group, a macrocyclic ring of about 13 to 25 atoms including at least two amide linkages, a proline-analogous group, and a connecting segment joining moieties on either side of the proline-analogous group. Methods of making the HCV protease-inhibitory compounds, methods of using the compounds, formulations of the compounds, and pharmaceutical combinations including the compounds, are provided.

### CROSS-REFERENCE TO RELATED APPLICATIONS

This application claims the priority of U.S. Provisional Applications Ser. Nos. 60/873,146, filed Dec. 6, 2006, and 60/917,314, filed May 10, 2007, which are incorporated by reference herein in their entireties.

### FIELD OF THE INVENTION

The present invention relates to novel macrocyclic compounds that are useful as protease inhibitors, particularly as inhibitors of serine proteases, and more particularly as inhibitors of the NS3 serine protease from hepatitis C virus. Because these inhibitors interfere with protease activity necessary for hepatitis C virus survival, the compounds find utility as antiviral agents, especially for treatment of hepatitis C virus infections.

### BACKGROUND OF THE INVENTION

Hepatitis C virus (“HCV”) is the causative agent for hepatitis C, a chronic infection characterized by jaundice, fatigue, abdominal pain, loss of appetite, nausea, and darkening of the urine. HCV, belonging to the hepacivirus genus of the Flaviviriae family, is an enveloped, single-stranded positive-sense RNA-containing virus. The long-term effects of hepatitis C infection as a percentage of infected subjects include chronic infection (55-85%), chronic liver disease (70%), and death (1-5%). Furthermore, HCV is the leading indication for liver transplant. In chronic infection, there usually presents progressively worsening liver inflammation, which often leads to more severe disease states such as cirrhosis and hepatocellular carcinoma.

The HCV genome (Choo et al., Science 1989, 244, 359-362; Simmonds et al., Hepatology 1995, 21, 570-583) is a highly variable sequence exemplified by GenBank accession NC_{—}004102 as a 9646 base single-stranded RNA comprising the following constituents at the parenthetically indicated positions: 5′ NTR (i.e., non-transcribed region) (1-341); core protein (i.e., viral capsid protein involved in diverse processes including viral morphogenesis or regulation of host gene expression) (342-914); E1 protein (i.e., viral envelope) (915-1490); E2 protein (i.e., viral envelope) (1491-2579); p7 protein (2580-2768); NS2 protein (i.e., non-structural protein 2) (2769-3419); NS3 protease (3420-5312); NS4a protein (5313-5474); NS4b protein (5475-6257); NS5a protein (6258-7601); NS5b RNA-dependent RNA polymerase (7602-9372); and 3′ NTR (9375-9646). Additionally, a 17-1(Dalton −2/+1 frameshift protein, “protein F”, comprising the joining of positions (342-369) with (371-828) may provide functionality originally ascribed to the core protein.

The NS3 (i.e., non-structural protein 3) protein of HCV exhibits serine protease activity, the N-terminus of which is produced by the action of a NS2-NS3 metal-dependent protease, and the C-terminus of which is produced by auto-proteolysis. The HCV NS3 serine protease and its associated cofactor, NS4a, process all of the other non-structural viral proteins of HCV. Accordingly, the HCV NS3 protease is essential for viral replication.

Several compounds have been shown to inhibit the hepatitis C serine protease, but all of these have limitations in relation to the potency, stability, selectivity, toxicity, and/or pharmacodynamic properties. Such compounds have been disclosed, for example, in published U.S. Patent Application Nos. 2004/0266731, 2002/0032175, 2005/0137139, 2005/0119189, and 2004/0077600A1, and in published PCT patent applications WO 2005/037214 and WO 2005/035525. Macrocyclic inhibitors of HCV have been disclosed by the inventors herein in patent application U.S. Ser. No. 60/883,946. Accordingly, a need exists for new compounds that are useful for inhibiting the serine protease of HCV.

### SUMMARY OF THE INVENTION

The present invention is directed to compounds of Formula I, the compounds being adapted to inhibit the viral protease NS3 of the Hepatitis C Virus (HCV), to the use of compounds of compounds of Formula I in the treatment of malconditions for which inhibition of HCV protease is medically indicated, such as in the treatment of HCV infections, and to pharmaceutical compositions and combinations including a compound of Formula I as defined herein. The compounds of Formula I are adapted to bind to, and thus block the action of, an HCV-encoded protease enzyme that is required by the virus for the production of intact, mature, functional viral proteins from the viral polyprotein as translated from the viral RNA, and therefore for the formation of infectious particles, and ultimately for viral replication. The compounds of the invention are mimics or analogs of the peptide domain immediately N-terminal of the substrate site where the viral protease cleaves its native substrate viral polyprotein, and are believed to bind to and inhibit the protease by virtue of this mimicry or analogy.

An embodiment of the present invention provides a compound of Formula (I):

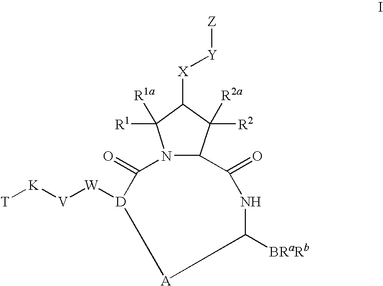

and stereoisomers, solvates, hydrates, tautomers, prodrugs, salts, pharmaceutically acceptable salts, and mixtures thereof, wherein:

R^{a} and R^{b} are independently a hydroxyl or a group that can be hydrolyzed to hydroxyl, or R^{a} and R^{b} together with the boron atom to which they are attached form a cyclic group which can be hydrolyzed to a B(OH)₂ group;

R¹, R^{1a}, R² and R^{2a} are independently H or a substituted or unsubstituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heteroaryl, or heteroarylalkyl group, wherein any carbon atom can be substituted with J;

D is CH₂, CH or N;

when D is CH₂, then W, V, K and T are absent;

when D is CH, then W is C(R⁶)₂, O, S, or NR⁷, and V, K, and T are as defined below;

when D is N then W, V and K are bonds, the bonds taken together forming a single bond, T is as defined below, such that T is bonded directly to D;

wherein R⁶ is independently at each occurrence hydrogen, alkyl, alkenyl, cycloalkyl, cycloalkylalkyl, aryl, aralkyl, heterocyclyl, heterocyclylalkyl, heteroaryl, or heteroarylalkyl, wherein any alkyl, alkenyl, aryl, aralkyl, heterocyclyl, heterocyclylalkyl, heteroaryl or heteroarylalkyl group is substituted with 0-3 J groups;

R⁷ is independently at each occurrence hydrogen, alkyl, cycloalkyl, cycloalkylalkyl, aryl, aralkyl, heterocyclyl, heterocyclylalkyl, heteroaryl, heteroarylalkyl, aralkanoyl, heteroaralkanoyl, C(O)R⁸, SO₂R⁸ or carboxamido, wherein any alkyl, aryl, aralkyl, heterocyclyl, heterocyclylalkyl, heteroaryl, heteroarylalkyl, aralkanoyl, or heteroaralkanoyl is substituted with 0-3 J groups;

R⁸ is alkyl, cycloalkyl, cycloalkylalkyl, aryl, aralkyl, heterocyclyl, heterocyclylalkyl, heteroaryl or heteroarylalkyl;

V is a bond,)C(R¹⁰)₂, C(O), S(O), or S(O)₂;

K is a bond, O, S, C(O), S(O), S(O)₂, S(O)(NR¹⁰)), or N(R¹⁰);

except when V and K are both bonds, the bonds taken together form a single bond;

R¹⁰ is independently at each occurrence hydrogen, alkyl, cycloalkyl, cycloalkylalkyl, aryl, aralkyl, heterocyclyl, heterocyclylalkyl, heteroaryl or heteroarylalkyl; wherein any R¹⁰ group except hydrogen is substituted with 0-3 J groups;

T is R¹¹, alkyl-R¹¹, alkenyl-R¹¹, alkynyl-R¹¹, OR¹¹, N(R¹¹)₂, C(O)R¹¹, or C(═NOalkyl)-R¹¹;

R¹¹ is independently hydrogen, alkyl, aryl, aralkyl, alkoxy, aryloxy, alkylamino, arylamino, cycloalkyl, cycloalkylidenyl, heterocyclyl, heterocyclylalkyl, heterocyclylalkylidenyl, heteroaryl, or heteroarylalkyl, wherein any R¹¹ except hydrogen is substituted with 0-3 J groups, or a first R¹¹ and a second R¹¹ together with a nitrogen atom to which they are bound form a mono- or bicyclic ring system substituted with 0-3 J groups;

J is halogen, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R, C(S)R′, C(O)OR′, OC(O)R′, C(O)N(R′)₂, OC(O)N(R′)₂, C(S)N(R)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein;

each R′ is independently at each occurrence hydrogen, (C₁-C₁₂)-alkyl, (C₃-C₁₀)-cycloalkyl or (C₃-C₁₀)-cycloalkenyl, [(C₃-C₁₀)cycloalkyl or (C₃-C₁₀)-cycloalkenyl]-(C₁-C₁₂)-alkyl, (C₆-C₁₀)-aryl, (C₆-C₁₀)-aryl-(C₁-C₁₂)-alkyl, (C₃-C₁₀)-heterocyclyl, (C₃-C₁₀)-heterocyclyl-(C₁-C₁₂)-alkyl, (C₅-C₁₀)-heteroaryl, or (C₅-C₁₀)-heteroaryl-(C₁-C₁₂)-alkyl, wherein R′ is substituted with 0-3 substituents selected independently from J; or, two R′ groups together with a nitrogen atom to which both R′ groups are attached or with two adjacent nitrogen atoms to which each R′ group is respectively attached form a mono- or bicyclic ring system;

A is a connecting segment comprising a chain of about 6 to about 17 carbon atoms comprising 0 or 1 double bond, wherein any chain carbon atom can bear a C₁-C₆ alkyl group, the chain further comprising 0-2 heteroatoms independently selected from O, S, S(O), S(O)₂, and NR⁷, the chain further comprising 0-3 J groups; and

when W is C(R⁶)₂, a bond, or absent:

X is a bond, O, S, C(R⁶)₂ or N(R⁷);

Y is a bond, C(R⁶)₂, C(O), C(O)C(O), S(O), S(O)₂, or S(O)(NR⁷);

except when X and Y are both bonds, the bonds taken together form a single bond;

Z is
a) hydrogen, alkyl, aryl, aralkyl, cycloalkyl, cycloalkylalkyl, heterocyclyl, heterocyclylalkyl, heteroaryl, heteroarylalkyl, OR⁹, or N(R⁹)₂, wherein any carbon atom is unsubstituted or is substituted with J, and wherein R⁹ is independently at each occurrence hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroarylalkyl, or two R⁹ groups which are bound to a nitrogen atom can form together with the nitrogen atom a 5-11 membered mono- or bicyclic heterocyclic ring system substituted with 0-3 J groups;
b) a substituted aryl or heteroaryl group; wherein any aryl or heteroaryl is substituted with 1-3 J groups;
c) a group of the formula:

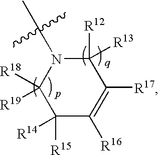

wherein R¹², R¹³, R¹⁴, R¹⁵, R¹⁸, and R¹⁹ are independently H, F, or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or R¹⁴ and R¹⁵ or R¹⁸ and R¹⁹, together with the carbon to which they are attached, can form a C₃₋₆ cycloalkyl group;

R¹⁶ and R¹⁷ are independently H or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹⁶ and R¹⁷ together with the atoms to which they are attached can form a fused substituted or unsubstituted aryl or heteroaryl group;

p is 0 or 1; and

q is 0 or 1;
d) a group of the formula:

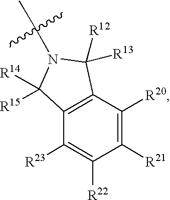

wherein R¹², R¹³, R¹⁴, and R¹⁵ are independently H, F, or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or R¹⁴ and R¹⁵, together with the carbon to which they are attached, can form a C₃₋₆ cycloalkyl group;

wherein R²⁰, R²¹, R²², R²³ are independently H, F, Cl, Br, I, CN, CF₃, OR²⁴, O—(CH₂)ᵣ—NR²⁵R²⁶, O—(CH₂)ᵣ—OC(O)NR²⁵R²⁶, O—(CH₂)ᵣ—NR²⁵C(O)OR²⁶, (CH₂)ᵣOR²⁴, OCF₃, NR²⁵R²⁶, (CH₂)ᵣNR²⁵R²⁶, SR²⁴, (CH₂)ᵣ—SR²⁴, C(O)R²⁴, C(O)OR²⁴, NR²⁷C(O)R²⁴, (C(O)NR²⁵R²⁶, NR²⁷C(O)NR²⁵R²⁶, OC(O)NR²⁵R²⁶, NR²⁷C(O)OR²⁴, NR²⁷SO₂R²⁴, SO₂NR²⁵R²⁶, or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group, wherein r is 1, 2, 3, 4, 5, or 6; and

each R²⁴, R²⁵, R²⁶, and R²⁷ is independently H or a substituted or unsubstituted alkyl, cycloalkyl, cycloalkenyl, cycloalkylalkyl, cycloalkenylalkyl, cycloalkylalkenyl, aryl, aralkyl, arylalkenyl, heterocyclyl, heterocyclylalkyl, heterocyclylalkenyl, heteroaryl, heteroarylalkyl, or heteroarylalkenyl group; or, when R²⁵ and R²⁶ are both bound to a nitrogen atom, R²⁵ and R²⁶ together with the nitrogen atom to which they are attached can form a 3-7 membered heterocyclic ring;
e) a group of the formula

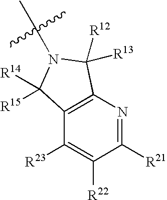

wherein R¹², R¹³, R¹⁴, R¹⁵, R²¹, R²² and R²³ are as defined above; or
f) a group of the formula

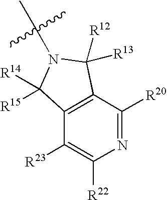

wherein R¹², R¹³, R¹⁴, R¹⁵, R²⁰, R²² and R²³ are as defined above;

wherein a wavy line signifies a point of attachment; or when W is NR⁷, O, or S:

X is O, CH₂, or NH;

Y is C(R⁶)₂ or absent;

Z is a substituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl; wherein any alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl is substituted with 1-3 J groups, provided that K and V are both bonds, taken together forming a single bond such that T is bonded directly to W, and T is not C(O)R¹¹; or

X is O;

Y is C(O); and

Z is:
aa) a group of the formula

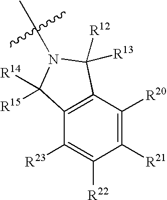

wherein R¹², R¹³, R¹⁴, R¹⁵, R²⁰, R²¹, R²² and R²³ are as defined above; or
bb) a group of the formula

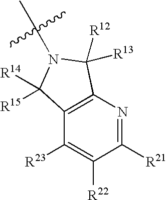

wherein R¹², R¹³, R¹⁴, R¹⁵, R²¹, R²² and R²³ are as defined above; or
cc) a group of the formula

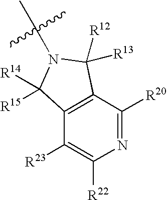

wherein R¹², R¹³, R¹⁴, R¹⁵, R²⁰, R²² and R²³ are as defined above; or

wherein a wavy line signifies a point of attachment.

An embodiment of the invention is directed to a method for synthesis of a compound of Formula I.

An embodiment of the invention is further directed to a pharmaceutical composition comprising a compound of Formula I and a suitable excipient.

An embodiment of the invention is further directed to a pharmaceutical combination comprising a compound of Formula I in a therapeutically effective amount and a second medicament in a therapeutically effective amount. The pharmaceutical combination of the invention may be formulated as a pharmaceutical composition of the invention.

An embodiment of the present invention is further directed to a method of treatment of a HCV infection in a patient in need thereof, or in a patient when inhibition of an HCV viral protease is medically indicated, comprising administering a therapeutically effective amount of a compound of Formula I to the patient, or a pharmaceutical combination to the patient.

### DETAILED DESCRIPTION OF THE INVENTION

### Definitions

The terms “HCV NS3 serine protease”, “HCV NS3 protease”, “NS3 serine protease”, and “NS3 protease” denote all active forms of the serine protease encoded by the NS3 region of the hepatitis C virus, including all combinations thereof with other proteins in either covalent or noncovalent association. For example, other proteins in this context include without limitation the protein encoded by the NS4a region of the hepatitis C virus. Accordingly, the terms “NS3/4a” and “NS3/4a protease” denote the NS3 protease in combination with the HCV NS4a protein.

The term “other type(s) of therapeutic agents” as employed herein refers to one or more antiviral agents, other than HCV NS3 serine protease inhibitors of the invention.

“Subject” as used herein, includes mammals such as humans, non-human primates, rats, mice, dogs, cats, horses, cows and pigs.

The term “treatment” is defined as the management and care of a patient for the purpose of combating the disease, condition, or disorder and includes administering a compound of the present invention to prevent the onset of the symptoms or complications, or alleviating the symptoms or complications, or eliminating the disease, condition, or disorder.

“Treating” within the context of the instant invention means an alleviation of symptoms associated with a disorder or disease, or inhibition of further progression or worsening of those symptoms, or prevention or prophylaxis of the disease or disorder. Thus, treating a hepatitis C viral infection includes slowing, halting or reversing the growth of the virus and/or the control, alleviation or prevention of symptoms of the infection. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result by inhibition of HCV NS3 serine protease activity. A therapeutically effective amount is also one in which any toxic or detrimental effects of compounds of the invention are outweighed by the therapeutically beneficial effects. For example, in the context of treating HCV infection, a therapeutically effective amount of a HCV NS3 serine protease inhibitor of the invention is an amount sufficient to control HCV viral infection.

All chiral, diastereomeric, racemic forms of a structure are intended, unless the specific stereochemistry or isomeric form is specifically indicated. Compounds used in the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these are all within the scope of the invention.

The term “amino protecting group” or “N-protected” as used herein refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc and Cbz. It is well within the skill of the ordinary artisan to select and use the appropriate amino protecting group for the synthetic task at hand.

In general, “substituted” refers to an organic group as defined herein in which one or more bonds to a hydrogen atom contained therein are replaced by a bond to a non-hydrogen atom such as, but not limited to, a halogen (i.e., F, Cl, Br, and I); an oxygen atom in groups such as hydroxyl groups, alkoxy groups, aryloxy groups, aralkyloxy groups; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, N-oxides, hydrazides, azides, and enamines; and other heteroatoms in various other groups. Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups may also be substituted with alkyl, alkenyl, and alkynyl groups as defined herein.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. Representative substituted alkyl groups may be substituted one or more times with any of the groups listed above, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which may be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic” and “carbocycle” denote a ring structure wherein the atoms of the ring are carbon. In some embodiments, the carbocycle has 3 to 8 ring members, whereas in other embodiments the number of ring carbon atoms is 4, 5, 6, or 7. Unless specifically indicated to the contrary, the carbocyclic ring may be substituted with as many as N-1 substituents wherein N is the size of the carbocyclic ring with for example, alkyl, alkenyl, alkynyl, amino, aryl, hydroxy, cyano, carboxy, heteroaryl, heterocyclyl, nitro, thio, alkoxy, and halogen groups.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C═C(CH₃), and —CH₂C≡C(CH₂CH₃) among others.

Aryl groups are cyclic aromatic hydrocarbons that do not contain heteroatoms. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halogen groups, bonded to one of the ring members. Rather, groups such as tolyl are referred to as substituted aryl groups. Representative substituted aryl groups may be mono-substituted or substituted more than once, such as, but not limited to, 2-, 3-, 4-, 5-, or 6-substituted phenyl or 2-8 substituted naphthyl groups, which may be substituted with carbon or non-carbon groups such as those listed above.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups include aromatic and non-aromatic ring compounds containing 3 or more ring members, of which, one or more is a heteroatom such as, but not limited to, N, O, and S. In some embodiments, heterocyclyl groups include 3 to 20 ring members, whereas other such groups have 3 to 15 ring members. The phrase “heterocyclyl group” includes fused ring species including those comprising fused aromatic and non-aromatic groups. The phrase also includes polycyclic ring systems containing a heteroatom such as, but not limited to, quinuclidyl. However, the phrase does not include heterocyclyl groups that have other groups, such as alkyl or halogen groups, including all the substituent groups listed above as well as any other chemically feasible groups, bonded to one of the ring members. Rather, these are referred to as “substituted heterocyclyl groups”. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups may be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups”. Representative substituted heteroaryl groups may be substituted one or more times with groups such as those listed above.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl; 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

More specifically, aryl and heteroaryl groups can include phenyl, isoindolidinyl, imidazolyl, oxazolyl, benzimidazolyl, and benzoxazolyl; wherein any aryl or heteroaryl can be unsubstituted, mono-substituted, or independently pluri-substituted, for example with J groups as defined herein.

Heterocyclylalkyl groups are alkyl groups as defined above in which a hydrogen or carbon bond of an alkyl group is replaced with a bond to a heterocyclyl group as defined above. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

Heteroarylalkyl groups are alkyl groups as defined above in which a hydrogen or carbon bond of an alkyl group is replaced with a bond to a heteroaryl group as defined above.

The term “alkoxy” refers to an oxygen atom connected to an alkyl group as defined above. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like.

The terms “aryloxy” and “arylalkoxy” refer to, respectively, an aryl group bonded to an oxygen atom and an aralkyl group bonded to the oxygen atom at the alkyl. Examples include but are not limited to phenoxy, naphthyloxy, and benzyloxy.

The term “amine” (or “amino”) includes primary, secondary, and tertiary amines having, e.g., the formula —NR₂. Amines include but are not limited to —NH₂, alkylamines, dialkylamines, arylamines, alkylarylamines, diarylamines, aralkylamines, heterocyclylamines and the like.

The term “amide” (or “amido”) includes C- and N-amide groups, i.e., —C(O)NR₂, and —NRC(O)R groups, respectively. Amide groups therefore include but are not limited to carbamoyl groups (—C(O)NH₂) and formamide groups (—NHC(O)H).

The term “urethane” (or “carbamyl”) includes N- and O-urethane groups, i.e., —NRC(O)OR and —OC(O)NR₂ groups, respectively.

The term “sulfonamide” (or “sulfonamido”) includes S- and N-sulfonamide groups, i.e., —SO₂NR₂ and —NRSO₂R groups, respectively. Sulfonamide groups therefore include but are not limited to sulfamoyl groups (—SO₂NH₂). An organosulfur structure represented by the formula —S(O)(NR)— is understood to refer to a sulfoximine, wherein both the oxygen and the nitrogen atoms are bonded to the sulfur atom, which is also bonded to two carbon atoms.

The term “amidine” or “amidino” includes groups of the formula —C(NR)NR₂. Typically, an amidino group is —C(NH)NH₂.

The term “guanidine” or “guanidino” includes groups of the formula —NRC(NR)NR₂. Typically, a guanidino group is —NHC(NH)NH₂.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

Without wishing to be bound by theory, the standard nomenclature of Schechter & Berger (Biochem. Biophys. Res. Comm., 1967, 27, 157-162) regarding the identification of residues in the polypeptide substrate of serine proteases will be employed herein unless other indicia of identification are specifically provided. Within the nomenclature of Schechter & Berger, the residues of the substrate, in the direction from the N-terminal toward the C-terminal, are labeled (Pi, . . . , P3, P2, P1, P1′, P2′, Pr′ . . . , Pj), wherein cleavage is catalyzed between P1 and P1′. Within the context of this nomenclature, compounds of Formulas I can be considered as mimics of at least the tripeptide P3-Pro-P1, wherein the analog of P1, as a moiety of the macrocyclic structure, is:

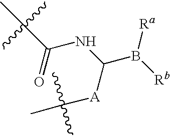

wherein R^{a} and R^{b} are as defined below and the wavy lines indicate points of attachment, and those points of attachment are ultimately connected to each other via a macrocyclic ring as described below.

An embodiment of the present invention is directed to a compound of Formula I:

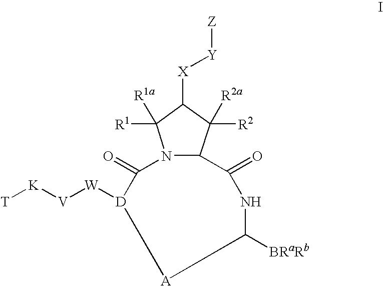

and stereoisomers, solvates, hydrates, tautomers, prodrugs, salts, pharmaceutically acceptable salts, and mixtures thereof, wherein the variables R^{a}, R^{b}, R¹, R^{1a}, R², R^{2a}, D, W, R⁶, R⁷, R⁸, V, K, R¹⁰, T, R¹¹, J, R′, A, X, Y, and Z, and further variables of structures comprised by Z, are as defined in the claims.

For example, R^{a} and R^{b} can each be hydroxyl such that the boron-containing group is a boronic acid. The boronic acid is believed to be a mimic of the carboxylic acid group of the native peptide substrate at the C-terminal segment of the enzyme binding site. Alternatively, R^{a} and R^{b}, and the boron atom to which they are attached, can together form a cyclic boronate ester, such as a boronate pinanediol ester. Or, the compound of Formula I can be a monobasic or dibasic salt of a boronic acid, wherein each corresponding cation is independently either a metallic or a non-metallic molecular entity.

An embodiment of the invention provides a compound of Formula I wherein D is CH₂ and W-K-V-T is absent. In another embodiment, D is N and V-K are a bond such that T is bonded directly to D. In another embodiment, D is CH, and W-V-K-T are as defined herein.

An embodiment of the invention provides a compound of Formula I wherein, when W is C(R⁶)₂, a bond, or absent, Z can be hydrogen, alkyl, aryl, aralkyl, cycloalkyl, cycloalkylalkyl, heterocyclyl, heterocyclylalkyl, heteroaryl, heteroarylalkyl, OR⁹, or N(R⁹)₂, wherein any carbon atom is unsubstituted or is substituted with J, and wherein R⁹ is independently at each occurrence hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroarylalkyl, or two R⁹ groups which are bound to a nitrogen atom can form together with the nitrogen atom a 5-11 membered mono- or bicyclic heterocyclic ring system substituted with 0-3 J groups. In another embodiment wherein when W is C(R⁶)₂, a bond, or absent, Z can be a group of the formula:

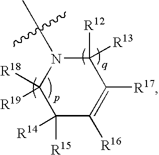

wherein R¹², R¹³, R¹⁴, R¹⁵, R¹⁸, and R¹⁹ are independently H, F, or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or ^{R}¹⁴ and R¹⁵ or R¹⁸ and R¹⁹, together with the carbon to which they are attached, can form a C₃₋₆ cycloalkyl group; R¹⁶ and R¹⁷ are independently H or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹⁶ and R¹⁷ together with the atoms to which they are attached can form a fused substituted or unsubstituted aryl or heteroaryl group; p is 0 or 1; and q is 0 or 1.

An embodiment of the invention provides a compound of Formula I, wherein Z, when when W is C(R⁶)₂, a bond, or absent, is a substituted aryl or heteroaryl group; wherein any aryl or heteroaryl can be substituted with 1-3 J groups.

An embodiment of the invention provides a compound of Formula I, wherein, when W is NR⁷, O, or S:

X is O, CH ₂, or NH;

Y is C(R⁶)₂ or absent;

Z is a substituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl; wherein any alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl is substituted with 1-3 J groups, provided that K and V are both bonds, taken together forming a single bond such that T is bonded directly to W, and T is not C(O)R¹¹; or; X is O; Y is C(O); and Z is: aa) a group of the formula

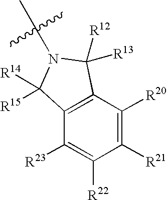

wherein R¹², R¹³, R¹⁴, R¹⁵, R²⁰, R²¹, R²² and R²³ are as defined above; or bb) a group of the formula

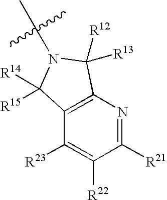

wherein R¹², R¹³, R¹⁴, R¹⁵, R²¹, R²² and R²³ are as defined above; or cc) a group of the formula

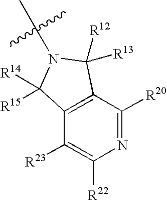

wherein R¹², R¹³, R¹⁴, R¹⁵, R²⁰, R²² and R²³ are as defined above; wherein a wavy line signifies a point of attachment.

Another embodiment of the invention provides a compound of Formula I, wherein Z, for any recited value of W, can be a group of the formula:

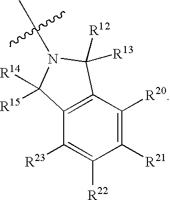

wherein R¹²-R¹⁵ and R²⁰-R²³ are as defined; and even more specifically, wherein R²⁰ is fluorine. For example, the invention provides a compound of Formula I wherein Z is a group of the formula:

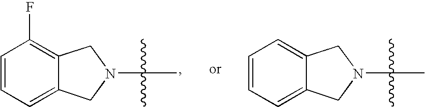

so a compound of the invention can include either of these groups linked by a —C(O)O— group (X═O, Y═C(O)) to the proline-analogous pyrrolidine ring. In the case of the second isoindoline structure of Z, all of R¹², R¹³, R¹⁴, R¹⁵, R²⁰, R²¹, R²² and R²³ are hydrogen, whereas in the case of the first, all these variables are H except R²⁰ is F.

Another embodiment of the invention provides a compound of Formula I, wherein Z, for any recited value of W, can be a group of the formula:

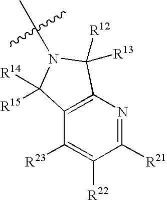

wherein R¹², R¹³, R¹⁴, R¹⁵, R²² and R²³ are as defined; or can be a group of the formula:

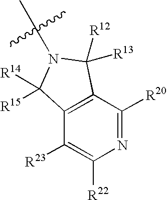

wherein R¹², R¹³, R¹⁴, R¹⁵, R²⁰, R²² and R²³ are as defined; wherein a wavy line signifies a point of attachment.

An embodiment of the invention further provides a compound of Formula I, wherein W is NR⁷, for example wherein W is NH. The invention further provides a compound of Formula I wherein V is also C(O), or wherein K is O, or wherein R¹¹ is alkyl or cycloalkyl, or any combination thereof. More specifically R¹¹ can be tert-butyl, or neopentyl, or cyclopentyl.

In an embodiment, T, defined as R¹¹, can be bonded directly to the NR⁷ group of W. For example, when W is NR⁷, X can be O, CH₂, or NH; Y can be C(R⁶)₂ or absent; and Z can be a substituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl; wherein any alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl is substituted with 1-3 J groups, provided that K and V are both bonds, taken together forming a single bond such that T is bonded directly to W, and T is not C(O)R¹¹.

In an embodiment of the invention, Z can be:

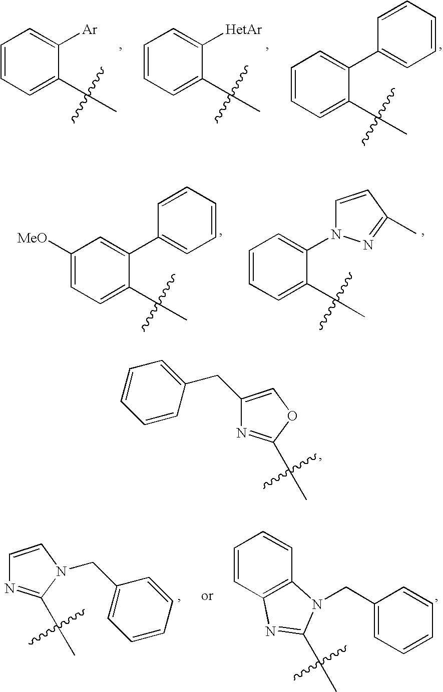

wherein a wavy line signifies a point of attachment, wherein any carbon atom of Z can be substituted with J, wherein Ar is substituted or unsubstituted aryl and HetAr is substituted or unsubstituted heteroaryl.

While the inventive compounds include all the stereoisomers of formula I, in an embodiment, the proline-analogous pyrrolidine ring is substituted with the carboxyl group and the 4-substituent (X-Y-Z) being disposed in a trans orientation on the ring, thus, a compound of Formula (IA):

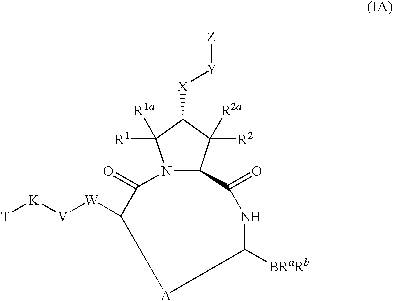

wherein the relative stereochemistry of the proline analog carboxyl group, and the proline analog 4-substituent, is trans. In an embodiment, both enantiomers of the compound of Formula (IA) are provided; in another embodiment a single enantiomer of the compound of Formula (IA) is provided; in another embodiment mixtures of the two enantiomers of the compound of Formula (IA) are provided wherein the two enantiomers are present in any possible ratio.

### Methods of Use

In one aspect, the invention provides methods of inhibiting HCV NS3 protease. The methods include contacting the hepatitis C viral serine protease with a compound as described herein. In other embodiments, the methods of inhibiting HCV NS3 protease include administering a compound as described herein to a subject infected with hepatitis C virus.

In another aspect, the invention provides methods for treating hepatitis C viral infection. The methods include administering to a subject in need of such treatment an effective amount of a compound of the invention as described herein. As used herein, “a compound” can refer to a single compound or a plurality of compounds. In some embodiments, the methods for treating hepatitis C viral infection include administering to a subject in need of such treatment an effective amount of a composition comprising a compound of the invention and a pharmaceutically acceptable carrier.

In another embodiment, the invention provides methods for treating hepatitis C viral infection comprising administering to a subject in need of such treatment an effective amount of a compound of the invention in combination with another anti-viral agent. The term “anti-viral agent” as used herein denotes a compound which interferes with any stage of the viral life cycle to slow or prevent HCV reproduction. Representative anti-viral agents include, without limitation, NS3 protease inhibitors, INTRON-A, (interferon alfa-2b available from Schering Corporation, Kenilworth, N.J.), PEG-INTRON (peginteferon alfa-2b, available from Schering Corporation, Kenilworth, N.J.), ROFERON-A (recombinant interferon alfa-2a available Hoffmann-La Roche, Nutley, N.J.), PEGASYS (peginterferon alfa-2a available Hoffmann-La Roche, Nutley, N.J.), INFERGEN A (Schering Plough, inteferon-alpha 2B+Ribavirin), WELLFERON (interferon alpha-n1), nucleoside analogues, IRES inhibitors, NS5b inhibitors, E1 inhibitors, E2 inhibitors, IMPDH inhibitors, NS5 polymerase inhibitors and/or NTPase/helicase inhibitors. In certain embodiments, the methods of treating HCV infection include administering to a subject in need of such treatment an effective amount of a compound of the invention in combination with another NS3 protease inhibitor. Examples of other NS3 protease inhibitors which can be administered in combination with compounds of the present invention include, without limitation, VX950 and BILN2061 (Lin C, Lin K, Luong Y, Rao B G, Wei Y Y, Brennan D L, Fulghum J R, Hsiao H M, Ma S, Maxwell J P, Cottrell K M, Perri R B, Gates C A, Kwong A D, “In Vitro Resistance Studies of Hepatitis C Virus Serine Protease Inhibitors VX950 and BILN2061”, J. Biol. Chem. (2004), 279, 17508-514).

Still other antiviral agents that may be used in conjunction with inventive compounds for the treatment of HCV infection include, but are not limited to, ribavirin (1-beta-D-ribofuranosy1-1H-1,2,-4-triazole-3-carboxamide, available from ICN Pharmaceuticals, Inc., Costa Mesa, Calif.; described in the Merck Index, entry 8365, Twelfth Edition); REBETROL® (Schering Corporation, Kenilworth, N.J.), COPEGASUS® (Hoffmann-La Roche, Nutley, N.J.); BEREFOR® (interferon alfa 2 available from Boehringer Ingelheim Pharmaceutical, Inc., Ridgefield, Conn.); SUMIFERON® (a purified blend of natural alpha interferons such as Sumiferon available from Sumitomo, Japan); ALFERON® (a mixture of natural alpha interferons made by Interferon Sciences, and available from Purdue Frederick Co., Conn.); .alpha.-interferon; natural alpha interferon 2a; natural alpha interferon 2b; pegylated alpha interferon 2a or 2b; consensus alpha interferon (Amgen, Inc., Newbury Park, Calif.); VIRAFERON®; INFERGEN®; REBETRON® (Schering Plough, Inteferon-alpha 2B+Ribavirin); pegylated interferon alpha (Reddy, K. R. et al. “Efficacy and Safety of Pegylated (40-kd) Interferon alpha-2a Compared with Interferon alpha-2a in Noncirrhotic Patients with Chronic Hepatitis C (Hepatology, 33, pp. 433-438 (2001); consensus interferon (Kao, J. H., et al., “Efficacy of Consensus Interferon in the Treatment of Chronic Hepatitis” J. Gastroenterol. Hepatol. 15, pp. 1418-1423 (2000); lymphoblastoid or “natural” interferon; interferon tau (Clayette, P. et al., “IFN-tau, A New Interferon Type I with Antiretroviral activity” Pathol. Biol. (Paris) 47, pp. 553-559 (1999); interleukin 2 (Davis, G. L. et al., “Future Options for the Management of Hepatitis C.” Seminars in Liver Disease, 19, pp. 103-112 (1999); Interleukin 6 (Davis et al. “Future Options for the Management of Hepatitis C.” Seminars in Liver Disease 19, pp. 103-112 (1999); interleukin 12 (Davis, G. L. et al., “Future Options for the Management of Hepatitis C.” Seminars in Liver Disease, 19, pp. 103-112 (1999); and compounds that enhance the development of type 1 helper T cell response (Davis et al., “Future Options for the Management of hepatitis C.” Seminars in Liver Disease, 19, pp. 103-112 (1999)). Also included are compounds that stimulate the synthesis of interferon in cells (Tazulakhova, E. B. et al., “Russian Experience in Screening, analysis, and Clinical Application of Novel Interferon Inducers” J. Interferon Cytokine Res., 21 pp. 65-73) including, but are not limited to, double stranded RNA, alone or in combination with tobramycin, and Imiquimod (3M Pharmaceuticals; Sauder, D. N. “Immunomodulatory and Pharmacologic Properties of Imiquimod” J. Am. Acad. Dermatol., 43 pp. S6-11 (2000)

In another embodiment, the invention provides a method for treating hepatitis C viral infection, comprising administering to a subject in need of such treatment an effective amount of a compound of the invention in combination with an anti-proliferative agent. The term “anti-proliferative agent” as used herein denotes a compound which inhibits cellular proliferation. Cellular proliferation can occur, for example without limitation, during carcinogenesis, metastasis, and immune responses. Representative anti-proliferative agents include, without limitation, 5-fluorouracil, daunomycin, mitomycin, bleomycin, dexamethasone, methotrexate, cytarabine, mercaptopurine.

In another embodiment, the invention provides a method for treating hepatitis C viral infection, comprising administering to a subject in need of such treatment an effective amount of a compound of the invention in combination with an immune modulator. The term “immune modulator” as used herein denotes a compound or composition comprising a plurality of compounds which changes any aspect of the functioning of the immune system. In this context, immune modulator includes without limitation anti-inflammatory agents and immune suppressants. Representative immune modulator include without limitation steroids, non-steroidal anti-inflammatories, COX2 inhibitors, anti-TNF compounds, anti-IL-1 compounds, methotrexate, leflunomide, cyclosporin, FK506 and combinations of any two or more thereof. Representative steroids in this context include without limitation prednisone, prednisolone, and dexamethasone. Representative non-steroidal anti-inflammatory agents in this context include without limitation ibuprofen, naproxen, diclofenac, and indomethacin. Representative COX2 inhibitors in this context include without limitation rofecoxib and celecoxib. Representative Anti-TNF compounds in this context include without limitation enbrel, infliximab, and adalumimab. Representative anti-IL-1 compounds in this context include without limitation anakinra. Representative immune suppressants include without limitation cyclosporin and FK506.

Compounds of the invention include mixtures of stereoisomers such as mixtures of diastereomers and/or enantiomers. In some embodiments, the compound, e.g. of Formula I, is 90 weight percent (wt %) or greater of a single diastereomer of enantiomer. In other embodiments, the compound is 92, 94, 96, 98 or even 99 wt % or more of a single diastereomer or single enantiomer.

A variety of uses of the invention compounds are possible along the lines of the various methods of treating a subject as described above. Exemplary uses of the invention methods include, without limitation, use of a compound of the invention in a medicament or for the manufacture of a medicament for treating a condition that is regulated or normalized via inhibition of the HCV NS3 serine protease.

### Biochemical Methods

Fluorescence resonance energy transfer (FRET; see e.g., Heim et al., (1996) Curr. Biol. 6:178-182; Mitra et al., (1996) Gene 173:13-17; and Selvin et al., (1995) Meth. Enzymol. 246:300-345) is an exquisitely sensitive method for detecting energy transfer between two fluorophoric probes. As known in the art, such probes are given the designations “donor” and “acceptor” depending on the relative positions of the maxima in the absorption and emission spectra characterizing the probes. If the emssion spectrum of the acceptor overlaps the absorption spectrum of the donor, energy transfer can occur. Because of the known and highly non-linear relationship of energy transfer and distance between fluorophores, approximated by an inverse sixth power dependence on distance, FRET measurements correlate with distance. For example, when the probes are in proximity, such as when the probes are attached to the N- and C-termini of a peptide substrate, and the sample is illuminated in a spectrofluorometer, resonance energy can be transferred from one excited probe to the other resulting in observable signal. Upon scission of the peptide linking the probes, the average distance between probes increases such that energy transfer between donor and accept probe is not observed. As a result, the degree of hydrolysis of the peptide substrate, and the level of activity of the protease catalyzing hydrolysis of the peptide substrate, can be quantitated. Accordingly, using methods known in the arts of chemical and biochemical kinetics and equilibria, the effect of inhibitor on protease activity can be quantitated.

Compounds of the invention will be found to have activity in this assay when employed to evaluate the inhibition of the HCV NS3 protease.

### Compositions and Combination Treatments

### A. Compositions.

Another aspect of the invention provides compositions of the compounds of the invention, alone or in combination with another NS3 protease inhibitor or another type of antiviral agent and/or another type of therapeutic agent. As set forth herein, compounds of the invention include stereoisomers, tautomers, solvates, prodrugs, pharmaceutically acceptable salts and mixtures thereof. Compositions containing a compound of the invention may be prepared by conventional techniques, e.g. as described in Remington: The Science and Practice of Pharmacy, 21st Ed., (2005). The compositions may appear in conventional forms, for example capsules, tablets, aerosols, solutions, suspensions or topical applications.

Typical compositions include a compound of the invention which inhibits the enzymatic activity of the HCV NS3 protease, and a pharmaceutically acceptable excipient which may be a carrier or a diluent. For example, the active compound will usually be mixed with a carrier, or diluted by a carrier, or enclosed within a carrier which may be in the form of an ampoule, capsule, sachet, paper, or other container. When the active compound is mixed with a carrier, or when the carrier serves as a diluent, it may be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration may be any route which effectively transports the active compound of the invention which inhibits the enzymatic activity of the HCV NS3 protease to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation may be tabletted, placed in a hard gelatin capsule in powder or pellet form or it can be in the form of a troche or lozenge. If a liquid carrier is used, the preparation may be in the form of a syrup, emulsion, soft gelatin capsule or sterile injectable liquid such as an aqueous or non-aqueous liquid suspension or solution.

Injectable dosage forms generally include aqueous suspensions or oil suspensions which may be prepared using a suitable dispersant or wetting agent and a suspending agent Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the formulation may also be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these. The compounds may be formulated for parenteral administration by injection such as by bolus injection or continuous infusion. A unit dosage form for injection may be in ampoules or in multi-dose containers.

The formulations of the invention may be designed to provide quick, sustained, or delayed release of the active ingredient after administration to the patient by employing procedures well known in the art. Thus, the formulations may also be formulated for controlled release or for slow release.

Compositions contemplated by the present invention may comprise, for example, micelles or liposomes, or some other encapsulated form, or may be administered in an extended release form to provide a prolonged storage and/or delivery effect. Therefore, the formulations may be compressed into pellets or cylinders and implanted intramuscularly or subcutaneously as depot injections or as implants such as stents. Such implants may employ known inert materials such as silicones and biodegradable polymers, e.g., polylactide-polyglycolide. Examples of other biodegradable polymers include poly(orthoesters) and poly(anhydrides).

For nasal administration, the preparation may contain a compound of the invention which inhibits the enzymatic activity of the HCV NS3 protease, dissolved or suspended in a liquid carrier, preferably an aqueous carrier, for aerosol application. The carrier may contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet that may be prepared by conventional tabletting techniques may contain:

| | |
|---|---|
| Core: | |
| Active compound (as free compound or salt thereof) | 250 mg |
| Colloidal silicon dioxide (Aerosil) ® | 1.5 mg |
| Cellulose, microcryst. (Avicel) ® | 70 mg |
| Modified cellulose gum (Ac-Di-Sol) ® | 7.5 mg |
| Magnesium stearate | Ad. |
| Coating: | |
| HPMC approx. | 9 mg |
| *Mywacett 9-40 T approx. | 0.9 mg |

*Acylated monoglyceride used as plasticizer for film coating.

A typical capsule for oral administration contains compounds of the invention (250 mg), lactose (75 mg) and magnesium stearate (15 mg). The mixture is passed through a 60 mesh sieve and packed into a No. 1 gelatin capsule. A typical injectable preparation is produced by aseptically placing 250 mg of compounds of the invention into a vial, aseptically freeze-drying and sealing. For use, the contents of the vial are mixed with 2 mL of sterile physiological saline, to produce an injectable preparation.

The compounds of the invention may be administered to a mammal, especially a human in need of such treatment, prevention, elimination, alleviation or amelioration of the various diseases as mentioned above, e.g., HCV infection. Such mammals include also animals, both domestic animals, e.g. household pets, farm animals, and non-domestic animals such as wildlife.

The compounds of the invention are effective over a wide dosage range. For example, in the treatment of adult humans, dosages from about 0.05 to about 5000 mg, preferably from about 1 to about 2000 mg, and more preferably between about 2 and about 2000 mg per day may be used. A typical dosage is about 10 mg to about 1000 mg per day. In choosing a regimen for patients it may frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge. HCV NS3 protease inhibitor activity of the compounds of the invention may be determined by use of an in vitro assay system which measures the potentiation of inhibition of the HCV NS3 protease. Inhibition constants (i.e., Kᵢ or IC₅₀ values as known in the art) for the HCV NS3 protease inhibitors of the invention may be determined by the method described in the Examples.

Generally, the compounds of the invention are dispensed in unit dosage form comprising from about 0.05 mg to about 1000 mg of active ingredient together with a pharmaceutically acceptable carrier per unit dosage.

Usually, dosage forms suitable for oral, nasal, pulmonal or transdermal administration comprise from about 125 μg to about 1250 mg, preferably from about 250 μg to about 500 mg, and more preferably from about 2.5 mg to about 250 mg, of the compounds admixed with a pharmaceutically acceptable carrier or diluent.

The invention also encompasses prodrugs of a compound of the invention which on administration undergo chemical conversion by metabolic or other physiological processes before becoming active pharmacological substances. Conversion by metabolic or other physiological processes includes without limitation enzymatic (e.g, specific enzymatically catalyzed) and non-enzymatic (e.g., general or specific acid or base induced) chemical transformation of the prodrug into the active pharmacological substance. In general, such prodrugs will be functional derivatives of a compound of the invention which are readily convertible in vivo into a compound of the invention. Conventional procedures for the selection and preparation of suitable prodrug derivatives are described, for example, in Design of Prodrugs, ed. H. Bundgaard, Elsevier, 1985.

In another aspect, there are provided methods of making a composition of a compound described herein comprising formulating a compound of the invention with a pharmaceutically acceptable carrier or diluent. In some embodiments, the pharmaceutically acceptable carrier or diluent is suitable for oral administration. In some such embodiments, the methods may further comprise the step of formulating the composition into a tablet or capsule. In other embodiments, the pharmaceutically acceptable carrier or diluent is suitable for parenteral administration. In some such embodiments, the methods further comprise the step of lyophilizing the composition to form a lyophilized preparation.

### B. Combinations.

The compounds of the invention may be used in combination with i) one or more other NS3 protease inhibitors and/or ii) one or more other types of antiviral agents (employed to treat viral infection and related diseases) and/or one or more other types of therapeutic agents which may be administered orally in the same dosage form, in a separate oral dosage form (e.g., sequentially or non-sequentially) or by injection together or separately (e.g., sequentially or non-sequentially).

Accordingly, in another aspect the invention provides combinations, comprising:
a) a compound of the invention as described herein; and
b) one or more compounds comprising:
   i) other compounds of the present invention
   ii) anti-viral agents including, but not limited to, other NS3 protease inhibitors
   iii) anti-proliferative agents
   iv) immune modulators.

Combinations of the invention include mixtures of compounds from (a) and (b) in a single formulation and compounds from (a) and (b) as separate formulations. Some combinations of the invention may be packaged as separate formulations in a kit. In some embodiments, two or more compounds from (b) are formulated together while a compound of the invention is formulated separately.

Combinations of the invention can further comprise a pharmaceutically acceptable carrier. In some embodiments, the compound of the invention is 90 wt % or more of a single diastereomer or single enantiomer. Alternatively, the compound of the invention can be 91, 92, 93, 94, 95, 96, 97, 98, or 99 wt % or more of a single diastereomer or single enantiomer.

The dosages and formulations for the other antiviral agent to be employed, where applicable, will be as set out in the latest edition of the Physicians' Desk Reference.

In carrying out the methods of the invention, a composition may be employed containing the compounds of the invention, with or without another antiviral agent and/or other type therapeutic agent, in association with a pharmaceutical vehicle or diluent. The composition can be formulated employing conventional solid or liquid vehicles or diluents and pharmaceutical additives of a type appropriate to the mode of desired administration. The compounds can be administered to mammalian species including humans, monkeys, dogs, etc. by an oral route, for example, in the form of tablets, capsules, granules or powders, or they can be administered by a parenteral route in the form of injectable preparations. The dose for adult humans is preferably between 10 and 1,000 mg per day, which can be administered in a single dose or in the form of individual doses from 1-4 times per day.

### Methods of Preparation

The invention also provides a method of preparing a compound of Formula (I) of the invention. For example, the invention provides a synthetic method for a compound of Formula (I) wherein the connecting segment A is a carbon chain, which can optionally contain heteroatoms or be substituted with alkyl or J groups, comprising a single double bond, comprising contacting a compound of Formula (II):

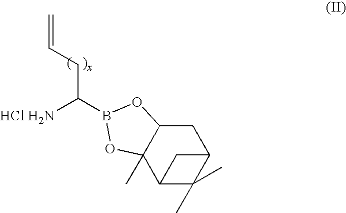

wherein x is 1 to about 7; and a compound of formula (III):

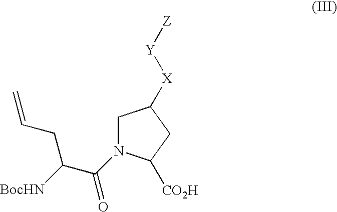

wherein X, Y, and Z are as defined herein, under conditions suitable to bring about formation of an amide bond; to provide a compound of Formula (IV):

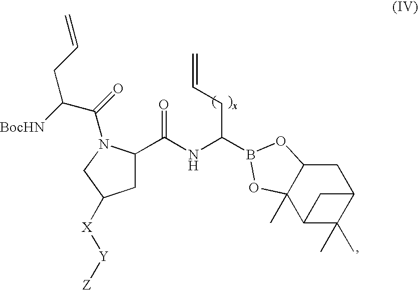

then, contacting the compound of Formula (IV) with a Ring-Closure-Metathesis (RCM) catalyst, for example a Grubbs-Hoveyda 1^{st} generation catalyst, to provide a compound of Formula (I) comprising a newly formed ethylenic bond, comprising:

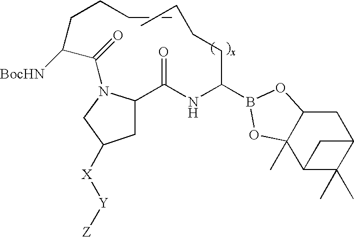

wherein the ethylenic bond can be cis or trans. A Grubbs-Hoveyda 1^{st} generation catalyst comprises ruthenium, being of the formula [RuCl(2)(=CH-(2-iPrO-) C(6)H(4))(IMesH(2))] where (IMesH(2)=1,3-bis(2,4,6-trimethylphenyl)-4,5-dihydroimidazol-2-ylidene).

It is within the knowledge of a person of ordinary skill to prepare a compound of Formula (I) with other forms of connecting segment A without undue experimentation, such as wherein A is a chain of about 6 to about 17 carbon atoms comprising 0 or 1 double bond, wherein any chain carbon atom can bear a C₁-C₆ alkyl group, the chain can further comprise 0-2 heteroatoms independently selected from O, S, S(O), S(O)₂, and NR⁷, the chain further comprising 0-3 7 groups; as defined herein. When the connecting segment A is a chain of 6 atoms, the entire macrocycle contains a total of 13 atoms; when the connecting segment A is a chain of 17 carbon atoms, the entire macrocycle contains a total of 24 atoms. In the condensation of the compounds of Formula (II) and Formula (III) and the subsequent ring closure metathesis reaction, a compound of Formula (I) wherein A is a linear alkyl chain with one double bond will be formed. A person of ordinary skill can, without undue experimentation, select the appropriate reagents in the preparation of the compounds of formula (II) and (III). Treatment of this double bond with hydrogen in the presence of a catalyst will provide a compound of Formula (I) wherein A is a linear alkyl chain with zero double bonds. For example, contacting a compound of Formula (I):

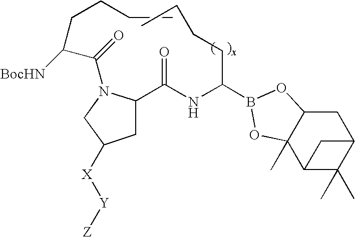

and hydrogen gas in the presence of a hydrogenation catalyst will provide a compound of Formula (I), comprising:

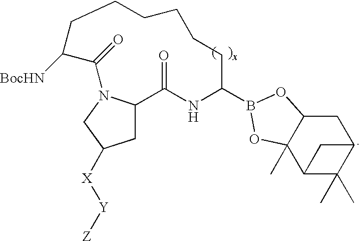

The compound of Formula (II) can be:

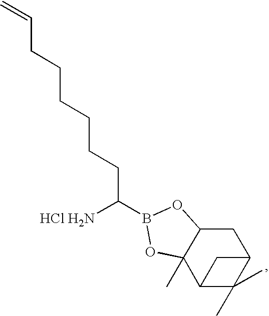

which can be prepared by contacting a compound of Formula (V):

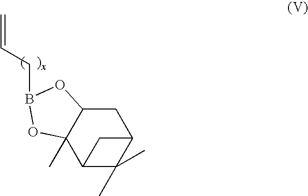

with dichloromethane and strong base to provide a compound of Formula (VI):

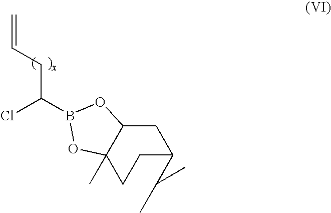

and then, contacting the compound of Formula (VI) and lithium hexamethyldisilazide, followed by aqueous hydrochloric acid, to provide the compound of Formula (11).

Thus, to prepare a compound of Formula (II) of the structure:

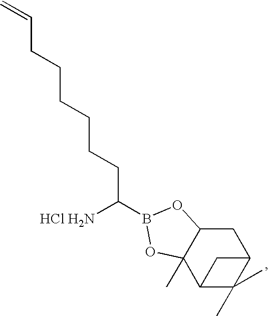

one can contact the compound of Formula (V) of the structure:

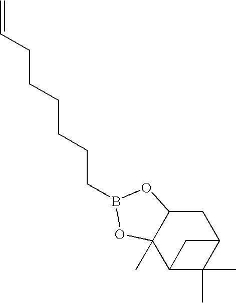

with dichloromethane and strong base, for example formed by contacting dichloromethane with n-butyllithium in THF at −78° C., to provide the compound of Formula (VI) of the structure:

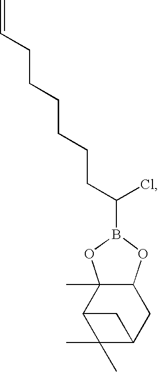

and then contacting this compound with lithium hexamethyldisilazide, then with aqueous hydrochloric acid, to provide a compound of the structure:

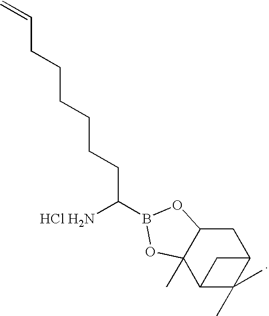

The compounds and processes of the present invention will be better understood in connection with the following examples, which are intended as an illustration only and not to limit the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

### Examples

The following abbreviations are used throughout this document.
BOP Benzotriazol-1-yl-oxy-tris-(dimethylamino)phosphonium hexafluorophosphate
CDI Carbonyl diimidazole
DBU Diazabicycloundecane
DCM Dichloromethane
DIEA, ⁱPr₂EtN N,N-Diisoproylethylamine
DMAP 4-(N,N-dimethylamino)pyridine
DMF N,N-Dimethylformamide
DMSO Dimethylsulfoxide
EDC 1-Ethyl-3-(3-dimethylaminopropyl)carbodiimide hydrochloride
eq Equivalents
Et₂0 Diethyl ether
EtOAc Ethyl acetate
h Hours
HATU O-(7-Azabenzotriazole-1-yl)-N,N,N′N′-tetramethyluronium hexafluorophosphate
HOAT Hydroxyazabenztriazole
HOBT Hydroxybenzotriazole
LiHDMS Lithium hexamethyldisilazide
mg Milligrams
min Minutes
mL Milliliters
μL Microliters
mmole Millimoles
MS Mass spectroscopy
MeOH Methanol
NaH Sodium hydride
NMM N-Methylmorpholine
rb Round-bottom
RT Room temperature
sat. Saturated
THF Tetrahydrofuran
˜ to (range, e.g., X˜Y═X to Y)

### General Procedures

### Example 1

General Procedure A (Ester Hydrolysis with LiOH):

To a solution of an ester (0.5 mmol) in a 3:2 mixture of THF/water (5 mL) is added LiOH (0.5 mmol) in one portion. After 3 h, a second portion of LiOH (0.1 mmol) is added. The reaction mixture is stirred for an additional hour. The reaction is then cooled down to 0° C. and 1N HCl (0.6 mmol) added dropwise over 2 min. The reaction is diluted with CH₂Cl₂ (10 mL) and washed with brine (5 mL). The organic layer is dried over Na₂SO₄ and evaporated under reduced pressure to yield an oily residue. A white solid precipitates from this oily residue upon standing overnight. This solid is washed with a 30% EtOAc/Hexanes mixture (2×5 mL) and used directly in the next reaction without further purification.

### Example 2

### General Procedure B (Amination):

To a −78° C. solution of the chlorocarbene homologation product (1.43 mmol) dissolved in THF (4 mL) under a balloon of dry N₂ is added LiHMDS (1.43 mL of a 1 M solution in THF) and the reaction allowed to warm to RT overnight. The THF is removed and dichloromethane added (˜30 mL) forming a white precipitate that is removed by filtration through a plug of Celite. The filtrate is concentrated to near dryness, cooled to −78° C. followed by addition of HCl (4 N HCl in dioxane, 1.5 mL), warmed to room temperature and concentrated to give a brown sticky solid.

### Example 3

### General Procedure C (Amide Formation)

To a solution of the substituted proline acid (0.396 mmol) in dry THF is added isobutylchloroformate (53 mL, 0.395 mmol) followed by N-methyl morpholine (86 mL, 0.414 mmol, 1.05 eq). Upon addition of the N-methyl morpholine a white precipitate immediately forms. The mixture is stirred for an additional 30 min followed by addition of the pinanediol protected amino-boronic acid(0.396 mmol) and N-methyl morpholine (86 μL, 0.414 mmol, 1.05 eq). The reaction is warmed to RT overnight, concentrated to near dryness and then diluted with dichloromethane (20 mL) and saturated NaHCO₃ solution (20 mL), then extracted with additional dichloromethane (10 mL). The organics are combined and washed with 0.5 N HCl (20 mL), brine (20 mL), dried over Na₂SO₄, concentrated in vacuo, and purified by flash column chromatography (silica gel, 2% MeOH in dichloromethane) to give the coupled product as a white solid.

### Example 4

### General Procedure D (Metathesis):

In a degassed solution of DCM (20 mL) was dissolved the di-alkene (0.129 mmol) and Grubbs-Hoveyda 1^{st} generation catalyst (6.44 !mole, 5% by wt.). The light yellow solution was heated at reflux for 18 h. The now dark reaction solution was concentrated and purified by flash column chromatography (silica gel, 2% MeOH in DCM) to afford the metathesis product in 63% yield.

### Example 5

### General Procedure E (Hydrogenation):

To a methanolic solution of alkene-macrocycle (0.04 mmol) was added catalyst Pd on C (10% by wt). The reaction flask was flushed several times with N₂ followed by careful addition of H₂ via balloon. The reaction was stirred for 30 min and then evacuated with excess N₂, filtered, concentrated and purified by flash chromatography (silica gel, eluted with 2% methanol in DCM) to afford the alkane macrocycle as a clear glassy solid.

### Example 6

### General Procedure F (Pinanediol Removal)

To a biphasic solution of acetonitrile and hexane was added the pinanediol ester (0.06 mmol, 1 eq), phenylboronic acid (0.12 mmol, 2 eq) and catalytic 1 N HCl (˜2 drops). The solution was stirred vigorously for 2 days during which time the hexane layer was regularly decanted and replaced with fresh hexane. The reaction was monitored by LCMS. Upon competition the acetonitrile layer was concentrated and purified by flash column chromatography (silica gel, 10% MeOH in DCM) to give the boronic acid.

### Specific Procedures

### Example 7

### 1,3-Dihydro-isoindole-2-carboxylic acid 1-(3-allyloxy-2-tert-butoxycarbonylamino-propionic)-5-carboxy-pyrrolidin-3-yl ester (5)

### A. (2S)-3-Allyloxy-2-tert-butoxycarbonylamino-propionic acid

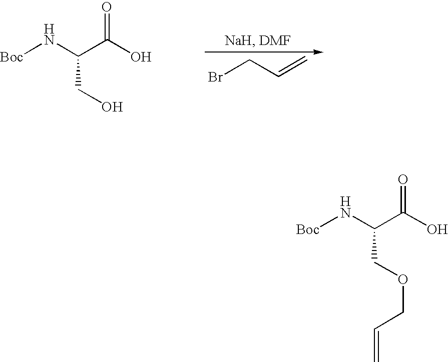

To a cooled (0° C.) solution of (2S)-3-hydroxy-2-tert-butoxycarbonylamino-propionic acid (1g, 4.87 mmol) dissolved in DMF (10 mL) under a blanket of N₂ gas was slowly added NaH (428 mg, 10.7 mmol, 60% dispersion in oil, 2.2 eq). The resulting solution was allowed to warm to RT over a 30 min period and then re-cooled to 0° C. Allyl bromide (453 μL, 5.36 mmol, 1.1 eq) dissolved in THF (2 mL) was added and the solution was allowed to warm to RT overnight. The now gelatinous reaction mixture was diluted with H₂O (25 mL) followed by Et₂O (25 mL). The H₂O layer was separated and the organics were washed with H₂O (2×15 mL). The aqueous layers were combined and further treated with Et₂O (2×20 mL) to remove residual DMF. Then, 1 N HCl (10.7 mL, 10.7 mmol, 2.2 eq) was added and the now cloudy white solution was extracted with EtOAc (3×30 mL). The organic extracts were combined and further washed with brine, dried over Na₂SO₄ and concentrated to give (2S)-3-allyloxy-2-tert-butoxycarbonylamino-propionic acid as an oily solid which was used directly without further purification.

### B. 2-Methoxycarbonyl-4-(1,3-Dihydroisoindolinyl-2-carboxy)pyrrolidine hydrochloride

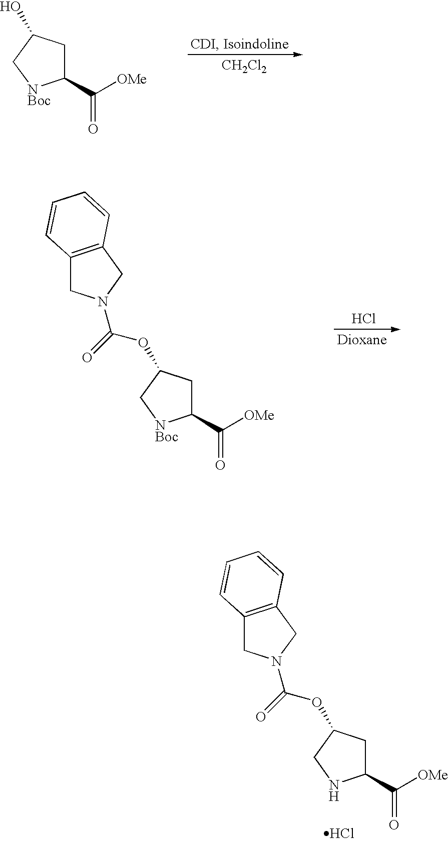

N-Boc-4-hydroxyproline methyl ester (397 mg, 1.6 mmol) was dissolved in CH₂Cl₂(10 mL) and CDI (315 mg, 1.9 mmol) was added in one portion at room temperature. The reaction mixture was stirred for 20 h. Isoindoline (0.55 ml, 4.8 mmol) was then added portion-wise over 8 h. After 20 h of additional stirring, the reaction was cooled down 0° C., diluted with CH₂Cl₂ (8 mL) and sequentially washed with aqueous 1N HCl (8 ml) and brine (8 ml). The organic layer was dried over Na₂SO₄ and evaporated under reduced pressure. The resulting oily residue was purified by silica gel column chromatography (solvent eluent gradient from 3:7 EtOAc/hexane to 6:4 EtOAc/hexane) to afford the intermediate N-Boc methyl ester (315 mg, 51%). MS m/z (rel intensity) 413 (M+23)⁺ (6), 291 (23), 128 (100). This product (315 mg, 0.81 mmol) was dissolved in 4N HCl in dioxane (8 mL). The reaction was stirred at room temperature for 1.5 h. Solvents were removed under reduced pressure to yield 2-methoxycarbonyl-4-(1,3 -dihydroisoindolinyl-2-carboxy)pyrrolidine hydrochloride as a white solid, which was used directly in the next reaction without further purification. MS m/z (rel. intensity) 291 (M+1)⁺ (4), 146 (17), 128 (100).

### C. 1-(3-Allyloxy-2-tert-butoxycarbonylamino-propionyl)-2-methoxycarbonyl-4-(1,3-dihydroisoindolinyl-2-carboxy)pyrrolidine

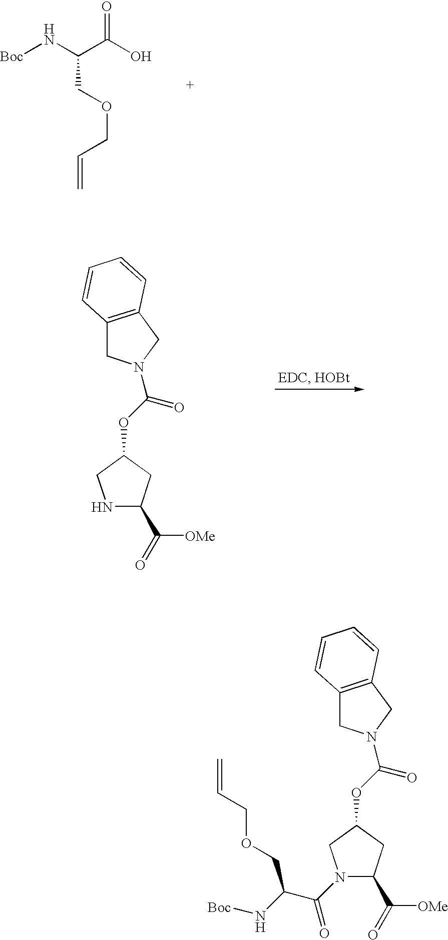

To solution of (2S)-3-allyloxy-2-tert-butoxycarbonylamino-propionic acid (525 mg, 2.14 mmol, 1.2 eq) in DCM (3 mL) was added EDC (538 mg, 2.8 mmol, 1.6 eq) and HOBt (379 mg, 2.8 mmol, 1.6 eq). After stirring at RT for 15 min the solution was cooled to 0° C. and a solution of 2-methoxycarbonyl-4-(1,3-dihydroisoindolinyl-2-carboxy)pyrrolidine hydrochloride (580 mg, 1.78 mmol, 1 eq) in DCM (2 mL) was slowly added, followed by NMM (700 μL, 4.7 mmol, 2.2 eq). The reaction was allowed to warm to RT over a 4 h period and was then quenched by addition of DCM (20 mL) and NaHCO₃ sat. solution (20 mL). The H₂O layer was extracted with additional DCM (2×15 mL), and the organics were combined and washed with 0.5 N HCl (20 mL), followed by NaHCO₃ sat. solution (20 mL), and finally with brine (20 mL). The organic layer was dried over Na₂SO₄ and concentrated to a thick oil. The oil was further purified by flash column chromatography (silica gel, eluted with a 1:2 hexane/EtOAc) to give 1-(3-allyloxy-2-tert-butoxycarbonylamino-propionyl)-2-methoxycarbonyl-4-(1,3-dihydroisoindolinyl-2-carboxy)pyrrolidine (704 mg, 1.36 mmol, 77% yield) as a sticky white solid.

### D. 1-(3-Allyloxy-2-tert-butoxycarbonylamino-propionyl)-2-carboxy-4-(1,3-dihydroisoindolinyl-2-carboxy)pyrrolidine (5)

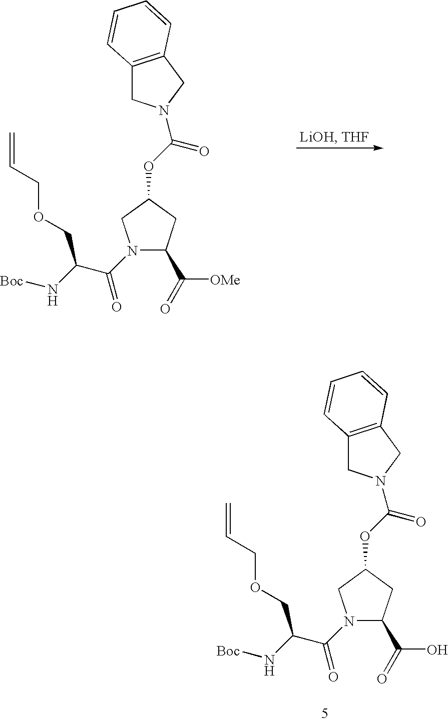

1-(3-Allyloxy-2-tert-butoxycarbonylamino-propionyl)-2-methoxycarbonyl-4-(1,3-dihydroisoindolinyl-2-carboxy)pyrrolidine was saponified with LiOH in THF/water solution according to general procedure A to yield compound 5, which was used without further purification.

### Example 8

### (+)-Pinanediol hex-5-ene-1-boronate

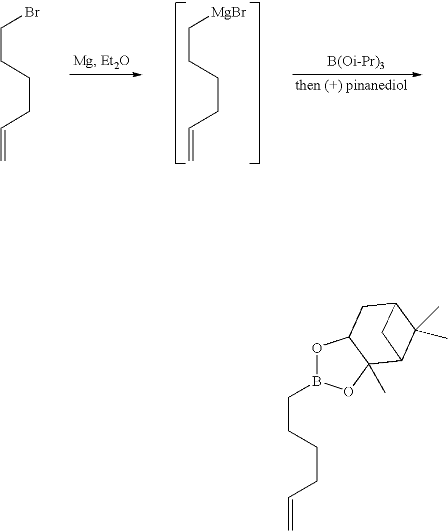

In a flame dried, 2-neck flask equipped with a reflux condenser and charged with dry N₂ gas was added Mg metal (353 mg, 14.72 mmol, 1.2 eq) and anhydrous Et₂O (10 mL). To this mixture was added slowly dripped 6-bromohex-1-ene (2g, 12.3 mmol) dissolved in dry Et₂O (8 mL). After approximately 25% of the bromohexene solution had been added the reaction was gently heated to reflux. The reflux was then kept constant by deliberate addition of the bromohexene solution. Upon complete addition the reaction was heated at reflux for 1 hr, cooled to RT, and added slowly to an ice cold ethereal solution of triisopropylborate (3.4 mL, 14.72 mmol, 1.2 eq). The reaction was allowed to warm to RT overnight followed by addition of 10% H₂SO₄ solution (20 mL) and additional Et₂O (40 mL). The biphasic solution was extracted, washed with NaHCO₃ sat. solution (40 mL), brine, dried over Na₂SO₄ and concentrated to approximately one half the original volume followed by addition of (+)-pinanediol (2.1 g, 12.3 mmol, 1 eq). After 4 h the solution was concentrated and purified by flash column chromatography (silica gel, eluted with 2% EtOAc in hexane) to afford (+)-pinanediol hex-5-ene-1-boronate (556 mg, 2.12 mmol, 18% yield) as a clear colorless liquid.

### Example 9

### (+)-Pinanediol (1R)-1-chloro-hept-6-ene-1-boronate

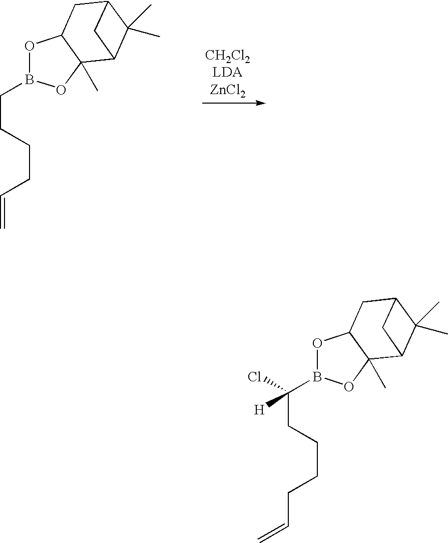

To a flame dried rb flask was added THF (7 mL), (+)-pinanediol hex-5-ene-1-boronate (1.0 g, 3.82 mmol), and DCM (490 μL, 7.64 mmol, 1.5 eq). The solution was cooled to −78° C. under dry N₂ (g) followed by addition of freshly prepared LDA (8.1 mL, 0.71 M solution in THF, 1.5 eq) over a 30 min period followed by ZnCl₂ (250 μL, 1.0 M solution in Et₂O, 0.65 eq). The reaction was allowed to slowly warm to 0° C. and then quickly quenched with H₂O (20 mL) and Et₂O (50 mL). The solution was extracted and the organic layer washed with 0.5 N HCl (30 mL), brine, dried over Na₂SO₄, and concentrated to give a viscous yellow oil. The oil was further purified by flash column chromatography (silica gel, eluted with 1% EtOAc in hexane) to afford (+)-pinanediol (1R)-1-chloro-hept-6-ene-1-boronate (627 mg, 2.12 mmol, 55% yield) as a 3:1 mixture of product and starting material.

### Example 10

### (+)-Pinanediol (1S)-1-amino-hept-6-ene-1-boronate hydrochloride

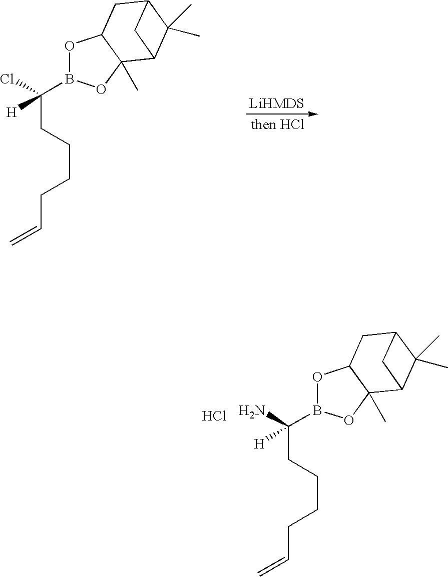

(+)-Pinanediol (1S)-1-amino-hept-6-ene-1-boronate hydrochloride was prepared from (+)-pinanediol (1R)-1-chloro-hepta-6-ene-1-boronate in a manner according to general procedure B and was used without further purification.

### Example 11

### Compound 8

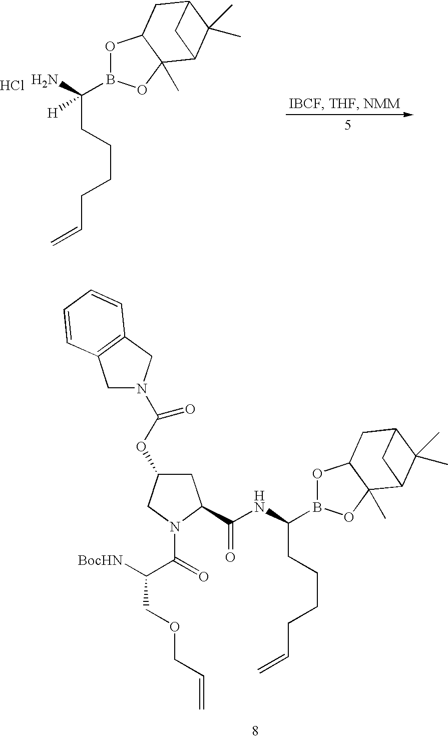

(+)-Pinanediol (1S)-1-amino-hept-6-ene-1-boronate hydrochloride was allowed to react with compound 5 in a manner according to general procedure C to afford 8 as a white sticky solid (417 mg, 0.54 mmol) in a 41% overall yield.

### Example 12

### Compound 9

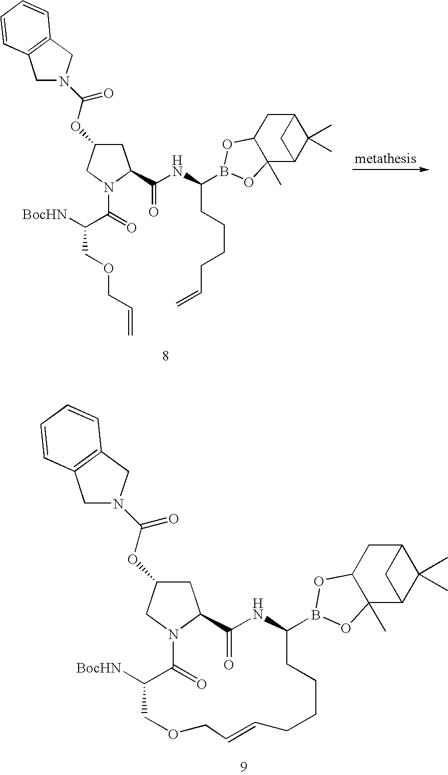

In a degassed solution of DCM (20 mL) was dissolved 8 (100 mg, 0.129 mmol) and Grubbs-Hoveyda 1^{st} generation catalyst (4 mg, 6.44 μmole, 5% by wt.). The light yellow solution was heated at reflux for 18 h. The now dark reaction solution was concentrated and purified by flash column chromatography (silica gel, 2% MeOH in DCM) to afford 9 (30.4 mg, 0.04 mmol) in 63% yield.

### Example 13

### Compound 10

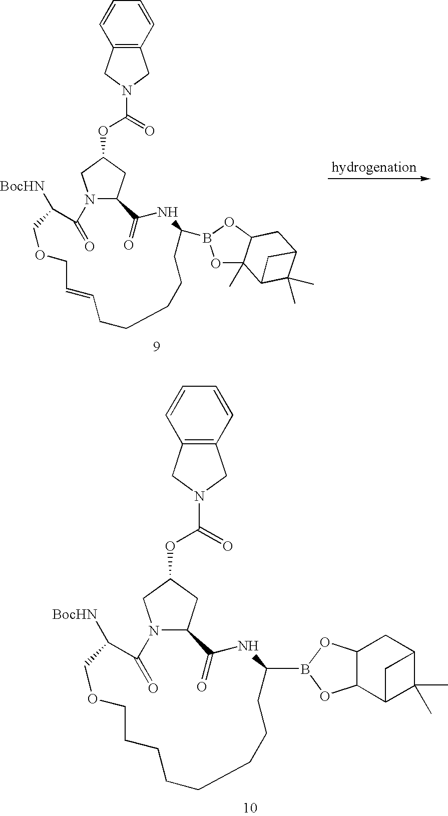

To a methanolic solution of 9 (30 mg, 0.04 mmol) was added catalyst Pd on C (10% by wt). The reaction flask was flushed several times with N₂ followed by careful addition of H₂ via balloon. The reaction was stirred for 30 min and then evacuated with excess N₂, filtered, concentrated and purified by flash chromatography (silica gel, eluted with 2% methanol in DCM) to afford 10 (14.7 mg, 0.019 mmol, 47%) as a clear glassy solid.

### Example 14

### Compound 11

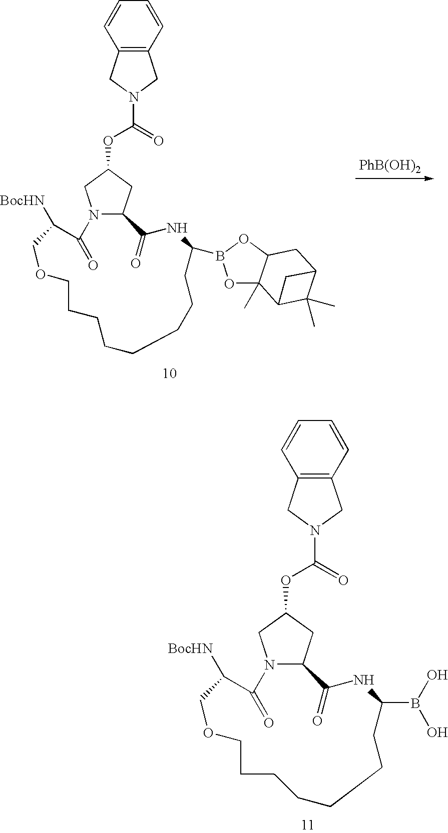

To a biphasic solution of acetonitrile and hexane was added 10 (50 mg, 0.06 mmol, 1 eq), phenylboronic acid (0.12 mmol, 14 mg, 2 eq) and catalytic 1 N HCl (˜2 drops). The solution was stirred vigorously for 2 days during which time the hexane layer was regularly decanted and replaced with fresh hexane. The reaction was monitored by LCMS. Upon competition the acetonitrile layer was concentrated and purified by flash column chromatography (silica gel, 10% MeOH in DCM) to give 11 (9 mg, 0.01 mmol, 17% yield).

### Example 15

### (+)-Pinanediol oct-7-ene-1-boronate

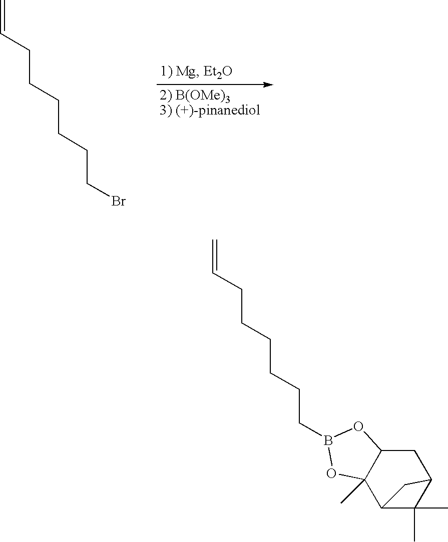

In a freshly cleaned, flame dried, 2-neck flask equipped with a reflux condenser and charged with dry N₂ gas was added Mg^{o} (1.4 g, 57 mmol, 1.1 eq) and anhydrous Et₂O (20 mL). To this mixture was added slowly dripped a solution of 8-bromooct-1-ene (10 g, 52.3 mmol) dissolved in dry Et₂O (10 mL). After approx. 25% of the ethereal solution had been added the reaction was gently heated to reflux. The refluxing solvent was then kept refluxing by deliberate addition of the bromide solution. Upon completion the reaction was heated at reflux for 1 h, cooled to rt, and added slowly to a −78° C. solution of trimethoxyborane (17.2 mL, 156 mmol, 3 eq) in diethyl ether. The reaction warmed to rt overnight and quenched by addition of a 10% H₂SO₄ solution (50 mL) and additional Et₂O (60 mL). The biphasic solution was extracted with addition Et₂O, washed with brine, dried over Na₂SO₄ and concentrated to approx ½ the original volume. To this was added (+)-pinanediol (8.94 g, 52.3 mmol, 1 eq) and after 2 h the solution was concentrated and purified by flash column chromatography (silica gel, eluted with 2% EtOAc in hexane) to afford (+)-pinanediol oct-7-ene-1-boronate (7.9 mg, 27.2 mmol, 52% yield) as a clear colorless oil.

### Example 16

### (+)-Pinanediol (1R)-1-chloro-non-8-ene-1-boronate

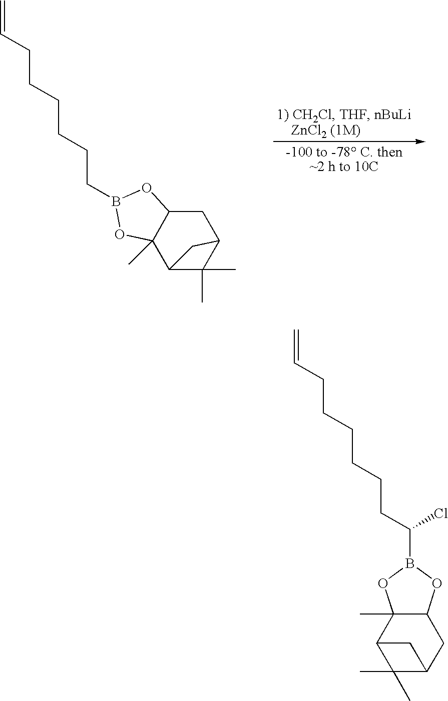

To a flame dried rb flask was added THF (10 mL) and DCM (1.75 mL, 27.5 mmol, 2.2 eq) and the solution was cooled in a liquid N₂/EtOH bath to −100° C. under a balloon of dry N₂ (g). N-BuLi (6 mL of a 2.5 M solution in hex, 1.2 eq) was added slowly over a 30 min period by running the solution down the cold side of the reaction flask. Upon competition the cooling bath was warmed to −78° C. by addition of dry ice and the reaction stirred for an additional 1 h. Then a solution of (+)-pinanediol oct-7-ene-1-boronate (3.62g, 12.5 mmol, 1 eq) in dry ether (10 mL) was added to the reaction over a 10 min period. After stirring at −78° C. for an addition 30 min ZnCl₂ (7.5 mL of a 1.0 M solution in ether) was added and the reaction was slowly warmed to 10° C. over a ˜2 h period then quenched with H₂O (40 mL), and 1 N HCl (˜5 mL), followed by additional Et₂O (50 mL). The organic layer was separated and the aqueous layer was washed with additional Et₂O. The organics were combined and washed with sat NaHCO₃, followed by brine, dried over Na₂SO₄ and concentrated to a thick yellow oil. Further purification by flash column chromatography (silica gel, eluent gradient from 1% EtOAc in hexane to 2.5% EtOAc in hexane) gave (+)-pinanediol(1R)-1-chloro-non-8-ene-1-boronate as a light colorless oil (3.36 g, 9.94 mmol of a 82:18 mixture of starting material and product, respectively, in 80% overall yield).

### Example 17

### (+)-Pinanediol (1 S)-1-amino-non-8-ene-1-boronate hydrochloride

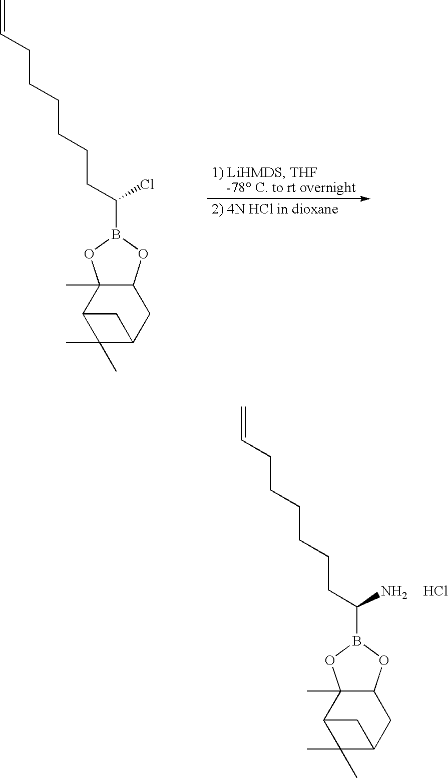

To a −78° C. solution of (+)-pinanediol (1R)-1-chloro-non-8-ene-1-boronate (8.15 mmol, 1 eq) in THF (8 mL) was added LiHMDS (8.5 mL of a 1M solution in THF, 8.5 mmol, 1.05 eq). The reaction was allowed to warm to rt overnight and then cooled again to −78° C. followed by addition of HCl (5.1 mL of a 4N solution in dioxane, 20.3 mmol, 2.5 eq). The cloudy yellow solution was warmed to rt, concentrated to dryness to give (+)-pinanediol (1S)-1-amino-non-8-ene-1-boronate hydrochloride as a thick viscous oil, which was used directly without further purification.

### Example 18

### Compound 15

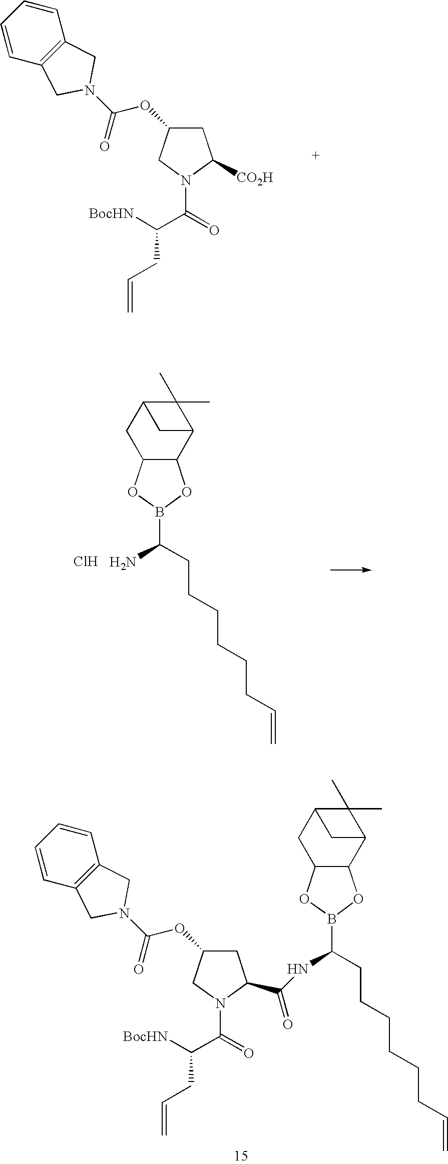

To a −10° C. solution of 14(2S)-2-tert-butoxycarbonylaminopent-4-enoyl)-2-carboxy-4-(1,3-dihydroisoindolinyl-2-carboxy)pyrrolidine (1.4 g, 2.96 mmol, 1.2 eq) and IBCF (418 μL, 3.2 mmol, 1.3 eq) was slowly added NMM (351 mL, 3.2 mmol, 1.3 eq) dissolved in THF (2 mL). The cloudy white solution was then warmed to rt for 20 min then cooled to −78° C. and (+)-pinanediol (1S)-1-amino-non-8-ene-1-boronate hydrochloride (2.27 mmol, 1 eq) dissolved in minimal DCM (˜5 mL) was added followed by an addition aliquot of NMM (272 μM, 2.47 mmol, 1 eq) dissolved in THF (˜2 mL) added slowly over a 20 min period. The reaction was allowed to warm to rt overnight. The solution was then evaporated to dryness and purified via flash column chromatography (silica gel, eluent gradient from 2% MeOH in DCM to 5% MeOH) gave 15 (1.36 g, 1.75 mmol, 71% yield) as an off-white solid.

### Example 19

### Olefin Metathesis of Compound 15

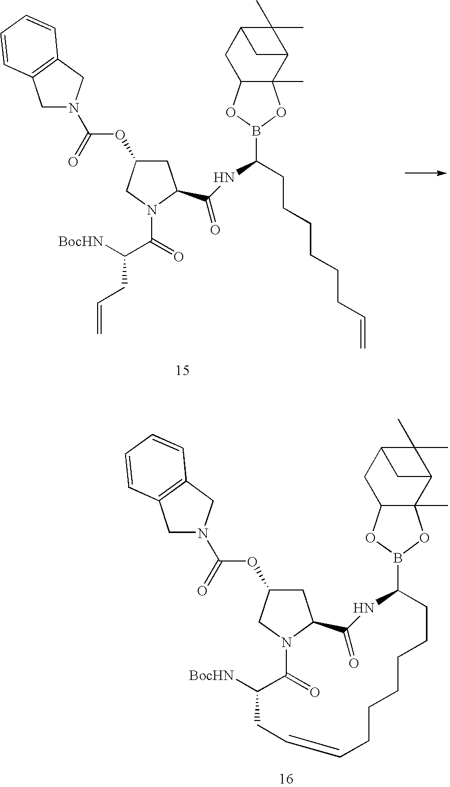

A solution of compound 15 (7 g, 9 mmol), 1,4-benzoquinone (97 mg, 0.09 mmol), and 2 drops of trifluoroacetic acid in toluene (6 mmol/L, 1.5 L) was purged with argon for 45 min. The reaction flask was then submerged in an oil bath which was heated to 80° C. When the reaction solution reached 62° C. to 65° C., the Grubbs catalyst 2^{nd} Generation (153 mg, 2 mol %) was added in one portion as a solid and the reaction stirred at this temperature. After 70 min, an additional 39 mg of the Grubbs catalyst 2^{nd} Generation was added in one portion as a solid. After 120 min, HPLC analysis indicated that the starting material was consumed. 2-Mercaptonicotinic acid (0.7 g, 4.5 mmol) was added to the reaction mixture, the heat was turned off in the oil bath, and the reaction mixture was stirred under argon for an additional 15 min. The flask was removed from the oil bath and the toluene was removed by distillation under reduced pressure to ˜¼ of the original volume (˜370 mL). The remaining mixture was washed with a 0.5 M NaHCO₃ solution (350 mL). After the layers were separated, the aqueous layer was washed with ethyl acetate (˜80 mL). The combined organic layers were treated with 2-mercaptonicotinic acid (0.7 g, 4.5 mmol) and activated charcoal (˜1 g) at room temperature for 1 h. The mixture was filtered through celite, washed with 0.5 M NaHCO₃ solution (2×350 mL), 1 N HCl (˜300 mL), brine (˜200 mL), dried with Na₂SO₄, filtered, and concentrated under reduced pressure. The resulting residue (7.55 g) was applied to silica gel (˜60 g) and eluted with a 5% to 40% ethyl acetate gradient in methylene chloride. The clean fractions, as indicated by HPLC analysis, were combined and concentrated to afford 5.13 g (76%) of the desired compound 16 as a white foamy solid.

### Example 20

### Hydrogenation of Compound 16

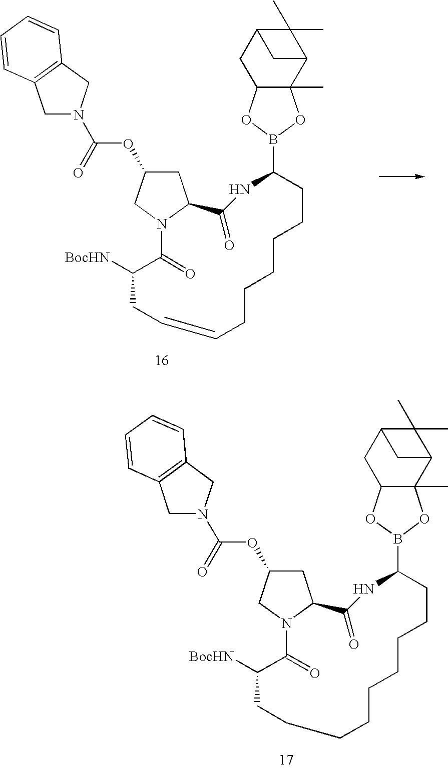

A solution of olefin 16 (1.34 g, 1.8 mmol) in MeOH (120 ml, 15 mM, HPLC grade) was purged with argon for 15 minutes. 10% Pd/C (267 mg, 20% by weight) was added in one portion and the system purged again with argon for 10 mM. A three way valve with a doubled hydrogen balloon was attached to the flask that was submitted to several cycles of vacuum/H₂. It was finally stirred under hydrogen for 75 min. LCMS showed complete conversion. The suspension was filtered through a Celite plug that was thoroughly washed with MeOH. The solvent was evaporated under reduced pressure to about 75 mL. The solution was then filtered through a fine fritted funnel to remove some leftover charcoal. After solvent removal, the white solid residue (compound 17) was used in the next step without further purification. The yield was 95% (1.29 g).

### Example 21

### Compound 18

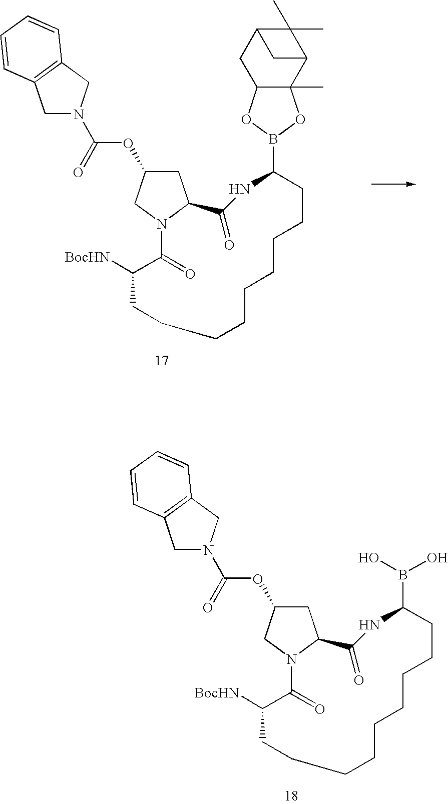

To a solution of the pinanediol boronic ester 17 (790 mg, 1.06 mmol) in MTBE (46 mL, anhydrous/new bottle) and CH₂Cl₂ (4.3 mL, anhydrous) at room temperature under Argon was sequentially added phenyl boronic acid (902 mg, 7 equiv.) and p-toluenesulfonic acid monohydrate (261 mg, 1.3 equiv.). The mixture was stirred for 16-17 hours (overnight under Ar) at which time no starting material was detected by LCMS analysis. The solution was then cooled down with an ice/water bath, diluted with MTBE (30 mL) and treated with a pre-cooled 5% NaHCO₃ aqueous solution (40 mL) in the same reaction flask with vigorous stirring for 5 min. After this time, brine (10 mL) was added and the mixture vigorously stirred again for 5 min. The mixture was transferred to a separation funnel. The aqueous layer was removed and the organic layer was washed a second time with 5% NaHCO₃ aqueous solution (40 mL). The organic layer was dried over Na₂SO₄, filtered and concentrated under reduced pressure. The white solid residue was purified by a short silica gel column chromatography (10×4 cm of silica gel) using mixtures of MeOH/CH₂Cl₂ as eluents. Appropriate fractions are pooled (checked by LCMS) and solvents removed. The residue is transferred to a smaller flask but filtering it first through a 0.2 μm PTFE filter to remove any leftover silica gel. The desired compound 18 (450 mg, 69% yield) was isolated as a solid.

TABLE 1
Exemplary Structures of the Invention

| Compound no. | Structure |
|---|---|
| 9 | 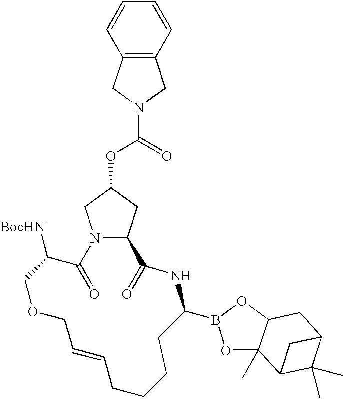 |
| 10 | 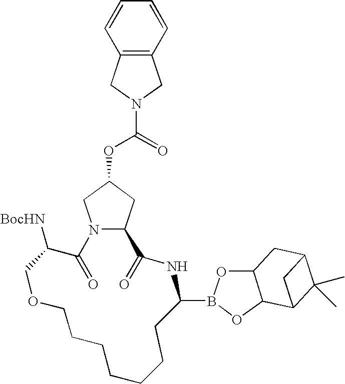 |
| 11 | 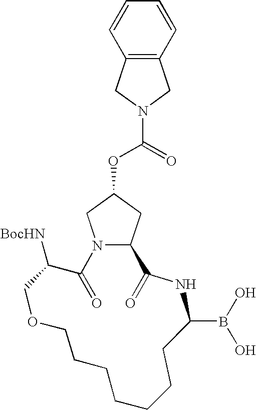 |
| 12 | 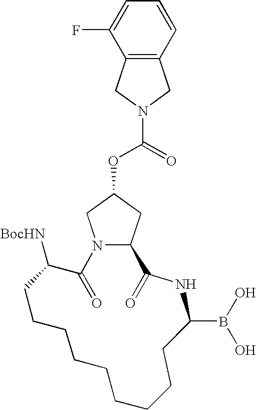 |
| 13 | 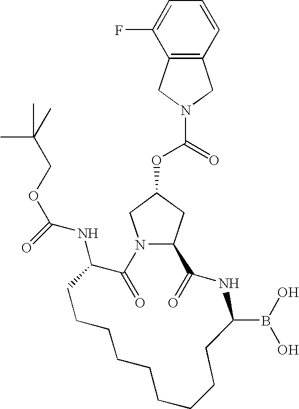 |
| 14 | 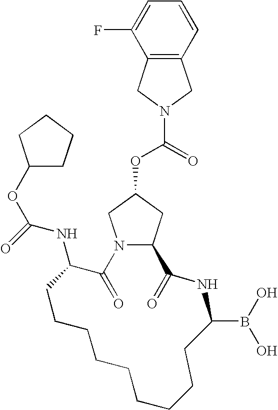 |
| 16 | 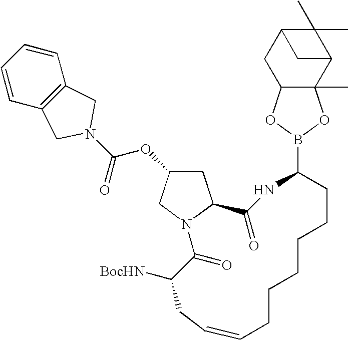 |
| 17 | 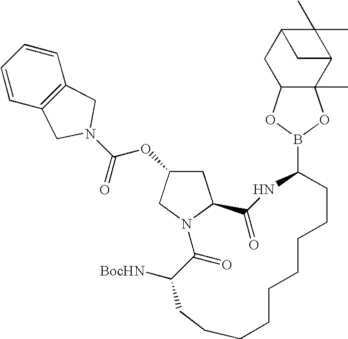 |
| 18 | 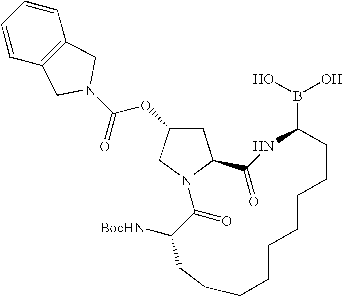 |

### Example 22

### Compound 19

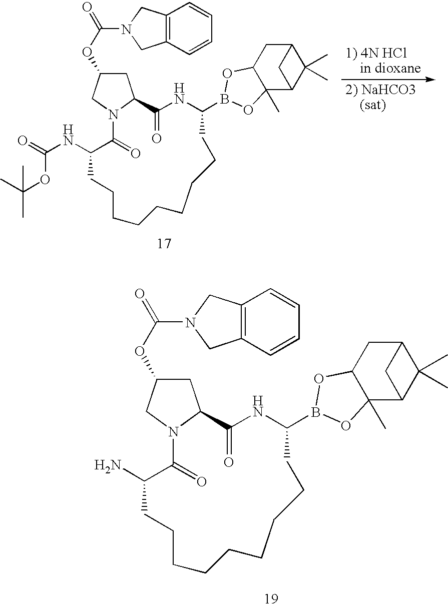

To solution of 17 (260 mg, 0.35 mmol) in DCM (3 mL) was added 4N HCl in dioxane (15 mL) and the resulting solution was allowed to stir at room temperature for 4 hr. The solvents were removed under reduced pressure and the resulting white solid was taken up in NaHCO₃ sat. solution and extracted with EtOAc (3×50 mL), washed with brine (25 mL), dried over Na₂SO₄ and concentrated under reduced pressure to give 19 (200 mg, 0.31 mmol, 89%) a thick viscous oil.

### Example 23

### Compound 20

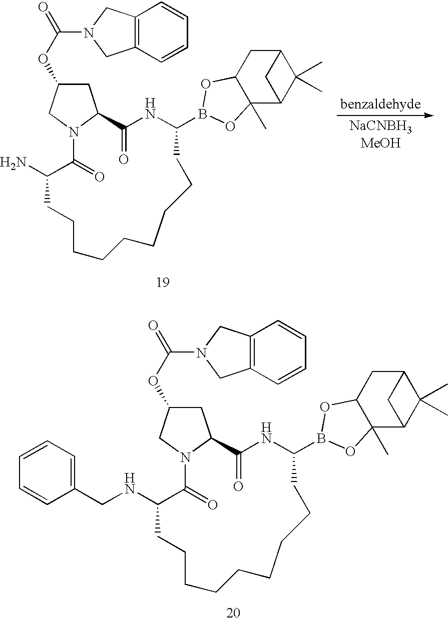

To a solution of 19 (260 mg, 0.35 mmol) dissolved in MeOH (3 mL) was added benzaldehyde (49 mL, 0.49 mmol, 1.6 eq), and NaCNBH₃ (38 mg, 0.62 mmol, 2 eq) at room temperature under a blanket of N₂. After 4 hr the reaction was quenched with H₂O (1 mL) and concentrated to near dryness under reduced pressure. The crude residue was taken up in NaHCO₃ sat solution (5 mL) and extracted with EtOAc (3×8 mL), and concentrated under reduced pressure to give a think dark oil. The crude 3 was further purified by flash column chromatography (silic gel, eluent gradient of 5%-10% MeOH in DCM) gave the 20 (76 mg, 0.11 mmol, 36% yield) as a while solid.

### Example 24

### Compound 21

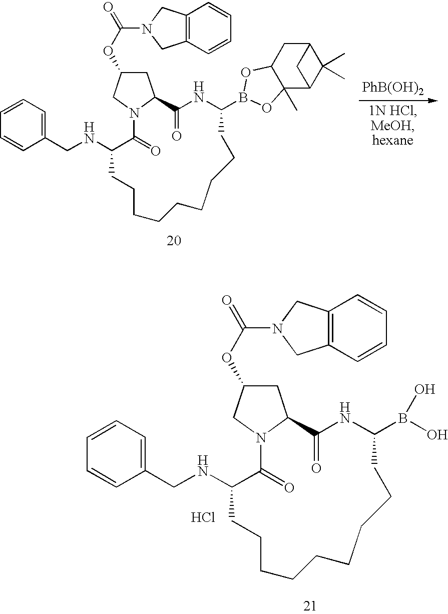

To a slightly cloudy solution of 20 (76 mg, 0.11 mmol) in 1:1 MeOH/1N HCl (2 mL) was added PhB(OH)₂ (13 mg, 0.12 mmol, 1.1 eq) followed by hexane (5 mL). The biphasic solution was stirred vigorously. After 18 1 hr the hexane layer was removed and fresh hexane was replaced and the solution was returned to vigorous stirring. After 3 additional cycles of hexane removal and replacement, the reaction was complete (as determined by LCMS analysis). The methanolic solution was evaporated to dryness and the solid residue was washed with 5 mL of Et₂O [to remove any residual PhB(OH)₂], dried under reduced pressure to give 21 (54 mg, 0.084 mmol, 77% yield) as a white free flowing powder.

### Compounds 22 to 43 Were Prepared According to the Procedure Outlined for the Synthesis of Compound 21.

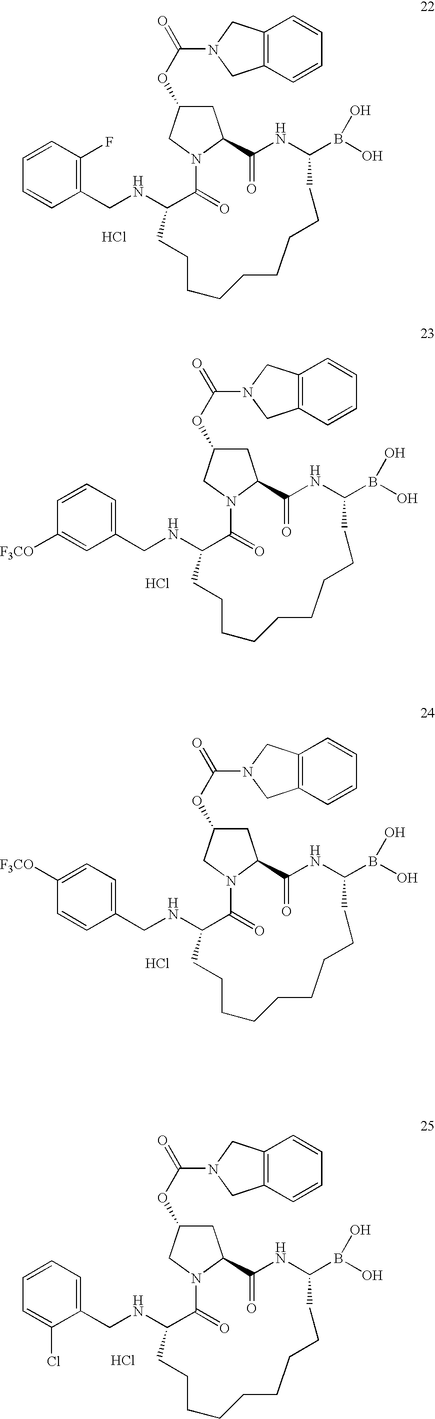

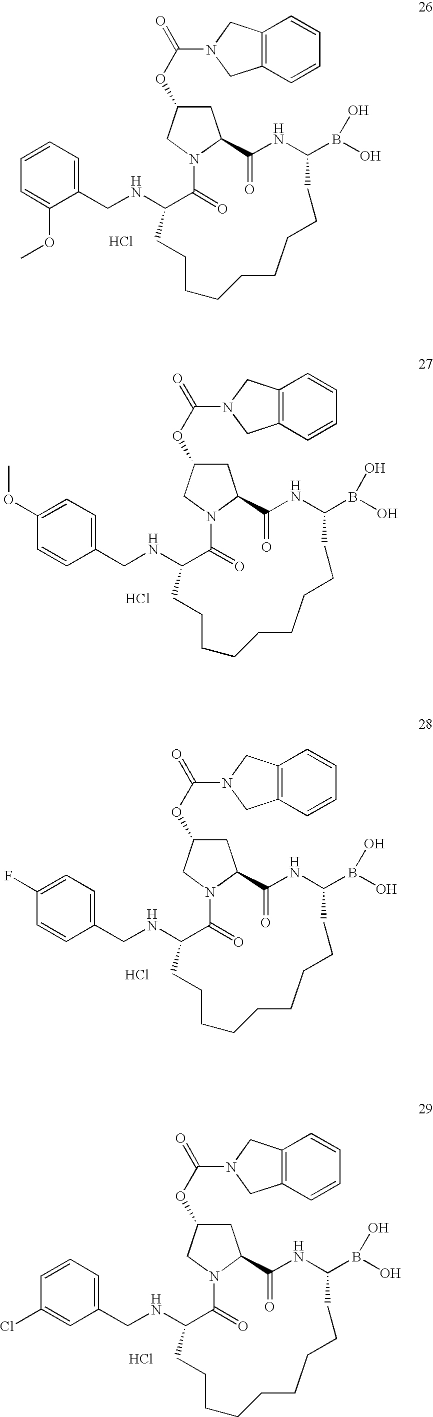

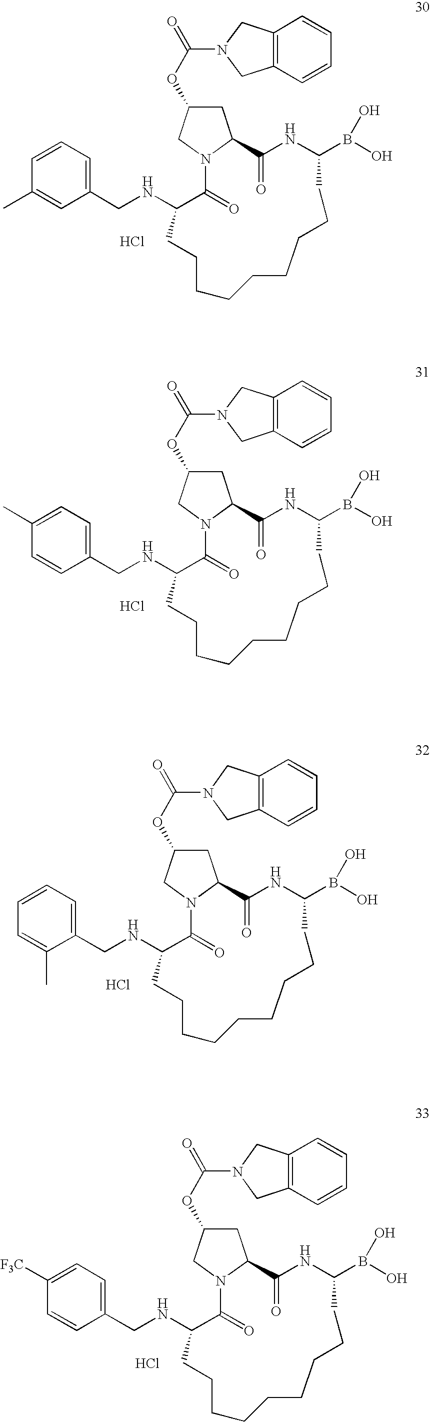

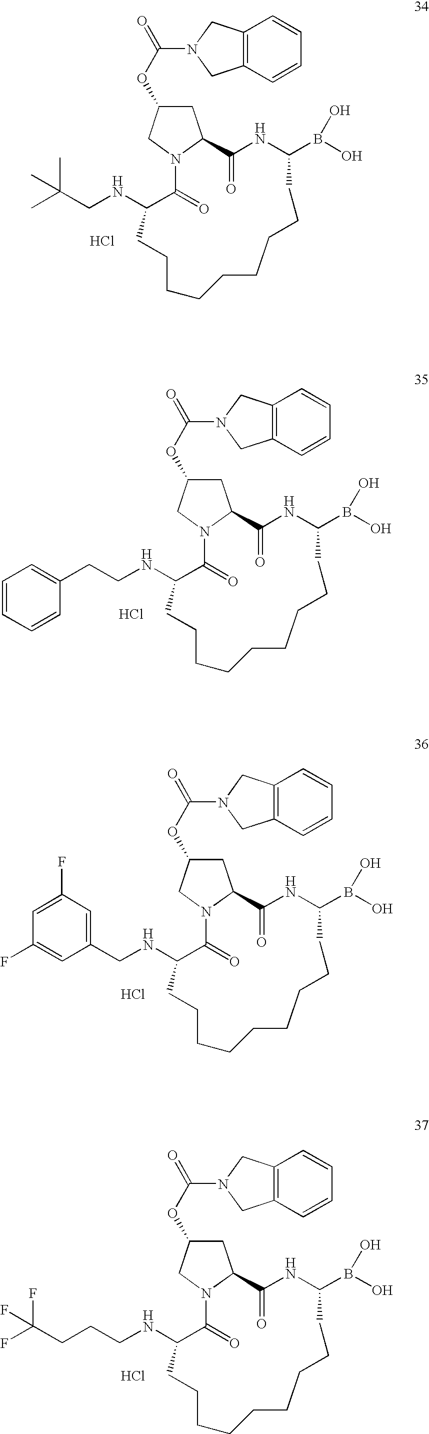

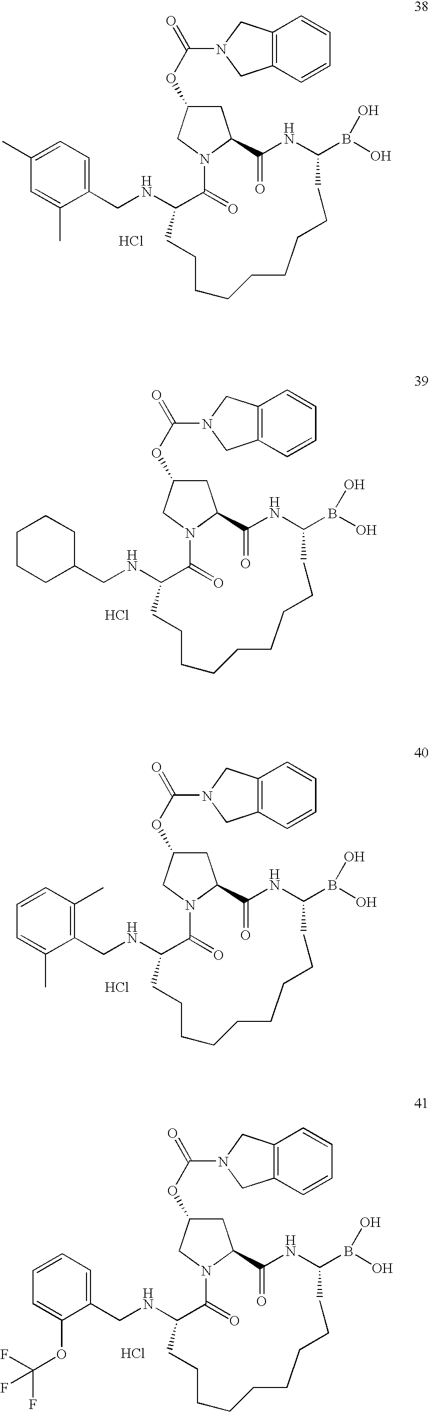

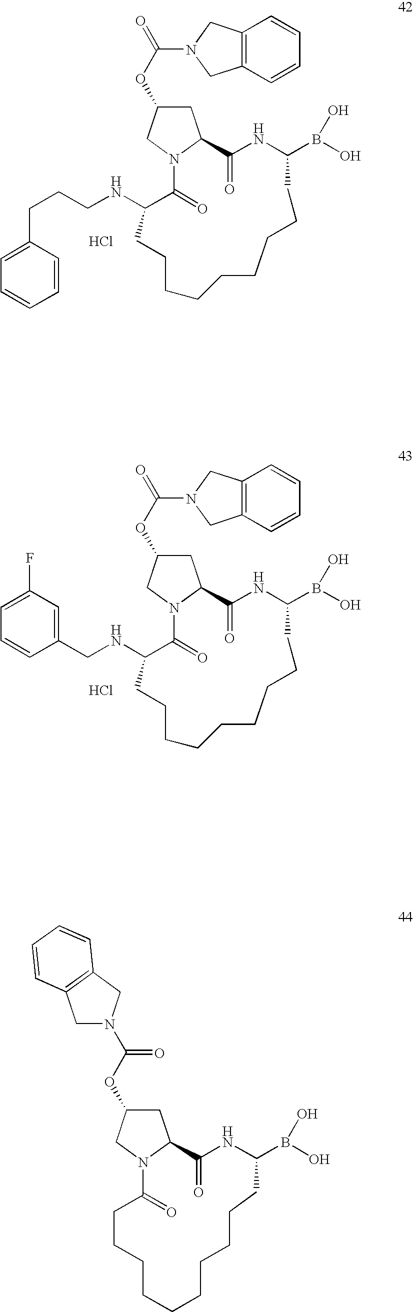

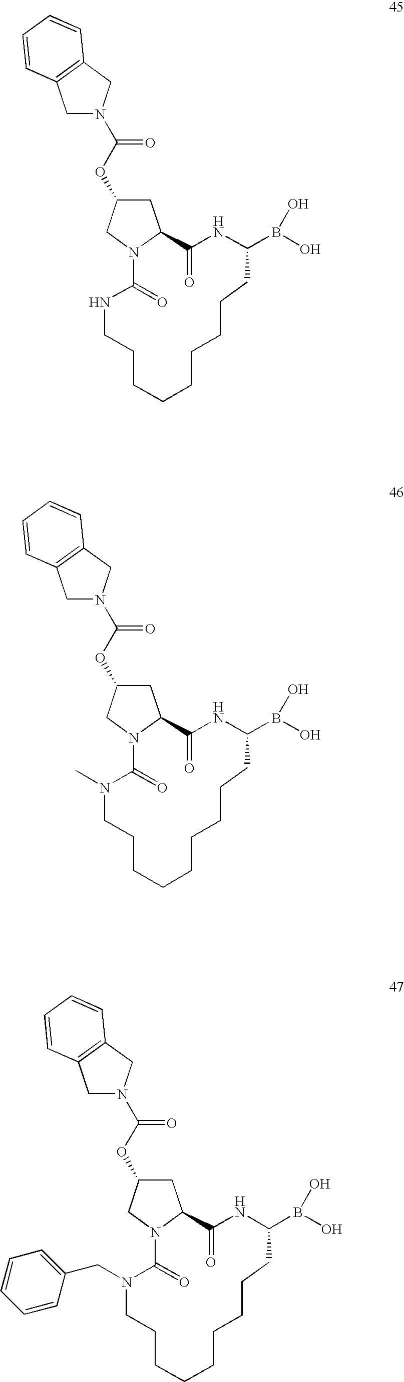

### Example 25

### Compound 44

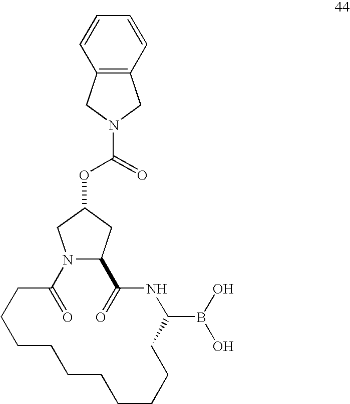

### Step 1

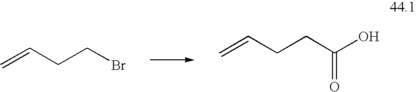

Mg (2.4 g, 100 mmol) and dry THF (100 mL) were introduced under N₂ atmosphere into a three-necked flask which was equipped with a dropping funnel and a thermometer. A solution of 4-bromo-1-butene (4.2 g, 30 mmol) in dry THF (100 mL) was introduced into the dropping funnel. About 10 ml of this solution was added first to trigger the reaction. The remaining solution was added dropwise while maintaining the temperature between 60° C.˜70° C. When the temperature of the reaction mixture reached room temperature, the reaction was finished.

Another three-necked flask with dropping funnel was introduced with nitrogen, after adding 50 mL of THF, CO₂ gas was introduced to the solution, after saturated with CO₂, the Grignard reagent was introduced to the dropping funnel. The reaction mixture was cooled to −40° C.˜−50° C., then Grignard reagent was dropped into the CO₂ solution over 1 hour. The resulting solution was stirred overnight. 1 N HCl (20 mL) was added to quench the reaction, the most solvent was removed under vacuum, the residue was extracted with EtOAc, dried over Na₂SO₄, evaporation to give the desired compound 1.1 g (yield: 35%).

### Step 2

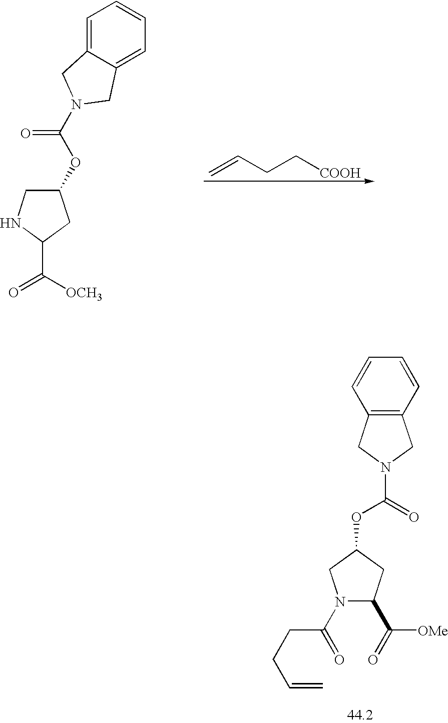

HATU (1.57 g, 4.1 mmol) was added to a solution of 2-methoxycarbonyl-4-(1,3-dihydroisoindolinyl-2-carboxy)pyrrolidine hydrochloride (1.2 g, 4.1 mmol) in 15 mL DCM, then 44.1 (0.28 g, 2.8 mmol) in 10 mL of DCM and DIPEA (3.56 g, 27.6 mmol) was added, the mixture was stirred at room temperature for 2 h. After completion the reaction, the mixture was concentrated to dryness, the residue was diluted with ethyl acetate, washed with 1 N HCl (30 mL), saturated NaHCO₃(30 mL) and brine(30 mL), dried over anhydrous Na₂SO₄, filtered and concentrated under vacuum, after evaporation of the solvent, the residue was purified by chromatography to give 44.2 as solid (1.17 g, 76%).

LC-MS (ESI): [M+H]⁺=373

### Step 3

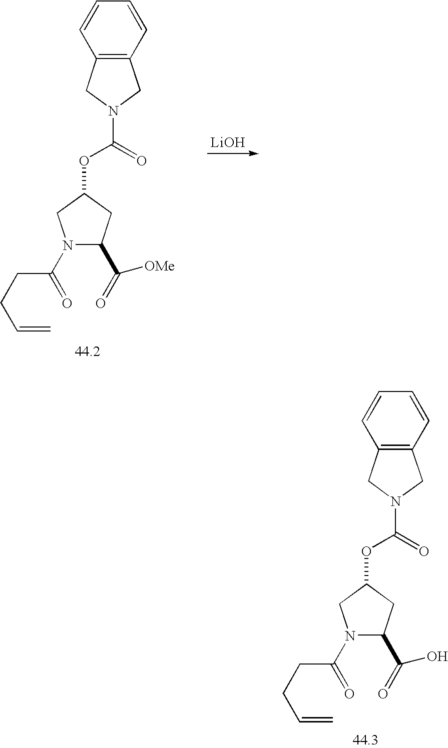

The compound of 44.2 (2.14 g, 5.74 mmol) was dissolved in 10 mL of THF. After adding 10 mL of aqueous LiOH (1N) solution, the resulted mixture was stirred for 4 h at room temperature. After completion of the reaction, most of THF was removed, the residue was adjusted to pH=3 with 1N HCl, and after extraction with DCM(30 mL×3), combined the organic phase, dried over anhydrous Na₂SO₄, and evaporated the solvent to give 44.3 as solid (1.87 g, 91%).

LC-MS (ESI): [M+H]⁺=359

### Step 4

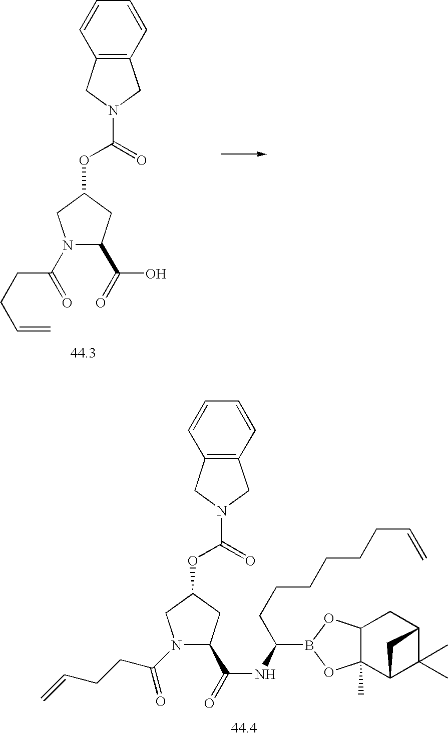

The suspension solution of 44.3 (1.85 g, 5.18 mmol) and HOBt (1.049 g, 7.76 mmol) in dichloromethane (20 mL) was cooled with an ice/water bath, then EDCI (1.488 g, 7.76 mmol) was added. After 30-45 minutes, the reaction mixture was cooled down to −15° C. to −10° C., (+)-pinanediol (1S)-1-amino-hept-6-ene-1-boronate hydrochloride (3.949 g, 11.1 mmol) was added, then a solution of DIPEA (2.0 g, 3eq.) in dichloromethane (1:4 v/v) was added over 30 minutes. The reaction mixture was stirred at room temperature for 2.5 hours, then 40 mL dichloromethane was introduced, the organic layer was washed with 1N HCl (50 mL), saturated NaHCO₃(50 mL), and brine(50 mL), dried over Na₂SO₄. After the removal of the solvent, the residue was purified by column chromatography twice (eluted with hexanes/EA(3:1˜2:1) to yield 1.5 g of 44.4 (62%). LC-MS (ESI): [M+H]⁺=660.

### Step 5

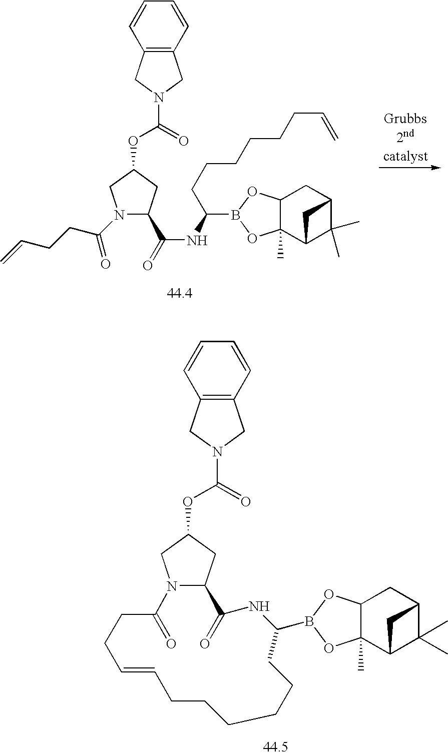

In a three necked round flask with condenser was added 44.4 (500 mg, 0.76 mmol), 1,4-benzoquinone (8.2 mg, 0.1 eqv.) and TFA 1% (mol/mol) in toluene (117 mL, 6.5 mmol/L), the resulted solution was degassed by bubbling argon into the solution at room temperature for 30 min. The flask was then placed in 65° C. oil bath and stirred with Argon bubbling into the solution and out of the flask, and then Grubbs 2^{nd} catalyst (19.3 mg, 3%) was added in three portions within 3 hours. HPLC indicated that the reaction was completed. The reaction mixture was cooled to 60° C. and 2-mercaptonicotinic acid (18 mg, 0.5 eqv.) was added in one portion. Toluene was removed by distillation under reduced pressure to around ⅓ of original volume. The organic phase was washed with 0.5 N NaHCO₃ (30 mL), the layers were separated and the organic layer was treated with 18 mg 2-mercaptonicotinic acid and 200 mg activated charcoal at room temperature for 1 hour. The mixture was filtered through Celite, washed with 0.5 N NaHCO₃ (30 mL), 1 N HCl solution (20 mL), and brine (20 mL). After drying with Na₂SO₄, filtered, after concentrating the filtrate in vacuo, the resulting residue is applied to silicon gel and eluted with 5% to 30% ethyl acetate gradient in methylene chloride to get 149 mg of 44.5. (Yield: 29%)

LC-MS (ESI): [M+H]⁺=632

### Step 6

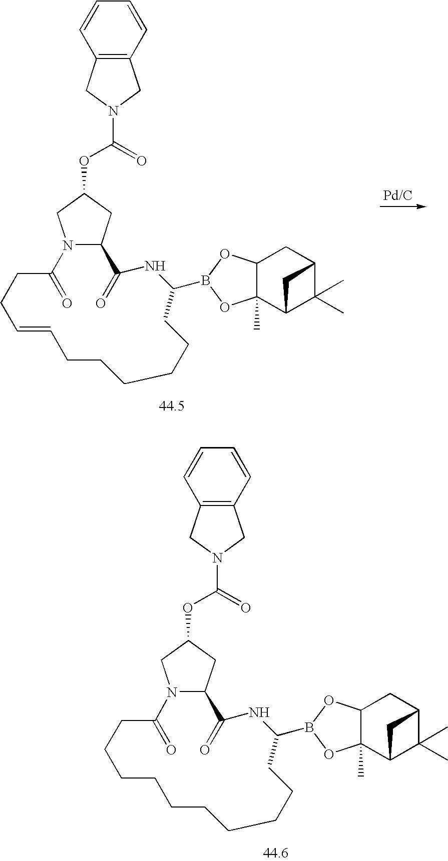

A solution of olefin 44.5 (149 mg, 0.23 mmol) in MeOH (5 ml) was purged with argon for 5 min. Pd/C (29.8 mg, 20% by weight) was added in one portion and the system was purged with argon for 5 min again. A three way valve with a doubled hydrogen balloon was attached to the flask that was submitted to several cycles of vacuum/H₂. It was finally stirred under hydrogen for 30 min. LCMS showed complete conversion. The catalyst was filtered off through a PTFE filter and the solvent was evaporated under vacuum to get 119 mg of 44.6 as white solid. (Yield: 80%)

LC-MS (ESI): [M+H]⁺=634

### Step 7

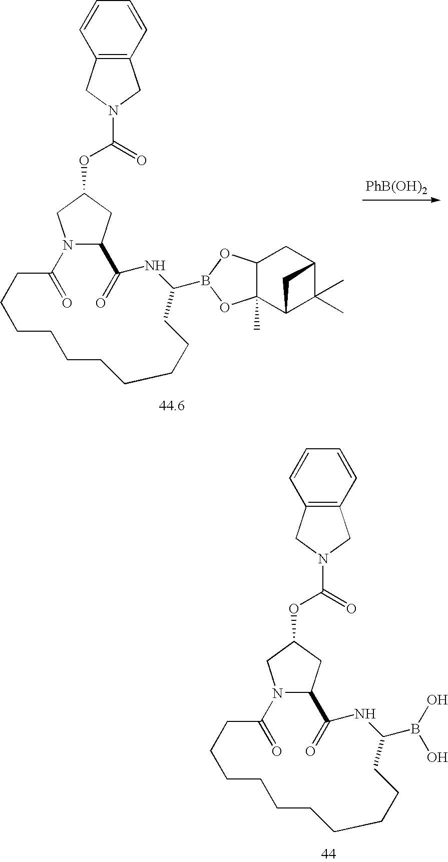

To a solution of the pinanediol ester 44.6 (114 mg, 0.1 mmol) in MTBE (11 mL, anhydrous/new bottle) and CH₂Cl₂ (1 mL, anhydrous) at room temperature under argon was sequentially added phenyl boronic acid (152 mg, 7 equiv.) and p-toluenesulfonic acid monohydrate (44 mg, 1.3 equiv.). The mixture was stirred for 16-17 hours (overnight under argon) at which time no starting material was detected by LCMS analysis. The solution was then cooled down with an ice/water bath, diluted with MTBE (3 mL) and washed with a precooled 5% NaHCO₃ aqueous solution (10 mL) in the same reaction flask with vigorous stirring. The aqueous layer was separated and the organic layer washed a second time with 5% NaHCO₃ aqueous solution (10 mL). The organic layer was dried over Na₂SO₄, filtered (dichloromethane was used to wash the sodium sulfate) and solvents were removed under reduced pressure. The white solid residue was purified by a short silica gel column chromatography using mixtures of MeOH/CH₂Cl₂ as eluents, initially with 3% MeOH/CH₂Cl₂ (around 100 ml, to remove the excess phenyl boronic acid and byproducts of the reaction) and then with increasing polarity (50 mL of 7, 11, 15, 20, 30, 40 and 50% MeOH/CH₂Cl₂, 100 mL of this last one). At last the column was washed with 60% MeOH/CH₂Cl₂. Appropriate fractions are pooled (checked by LCMS, filter samples through PTFE filter) and solvents removed. The residue is transferred (by using dichloromethane) to a smaller flask but filtering it through a 0.2 μm PTFE filter to remove any leftover silica gel. The final compound has a light yellow color. 20 mg of 44 was obtained.

LC-MS (ESI): [M-17]⁺=482

### Example 26

### Compound 45

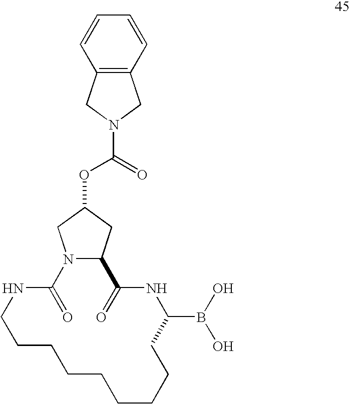

### Step 1

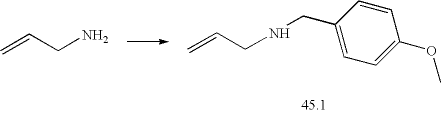

4-methoxybenzaldehyde (13.6 g, 100 mmol), prop-2-en-1-amine (5.7 g, 100 mmol) and methanol (100 mL) were introduced under N₂ atmosphere into a three-necked flask, the resulting solution was stirred at rt for 30 min, a few drops of concentrated HCl was introduced, then NaBH₄ (10.8 g, 300 mmol) in methanol (20 mL) was added, the resulting solution was stirred at room temperature overnight. After completion the reaction, the methanol was evaporated under vacuum, 100 mL water was added, EtOAc (100 mL×3) was used to extract, the organic layers were combined and dried over Na₂SO₄, the solvent was removed, and the residue was chromatographed with DCM/methanol (50:1) to give 8.85 g (yield: 50%) of 45.1 as yellowish oil.

### Step 2

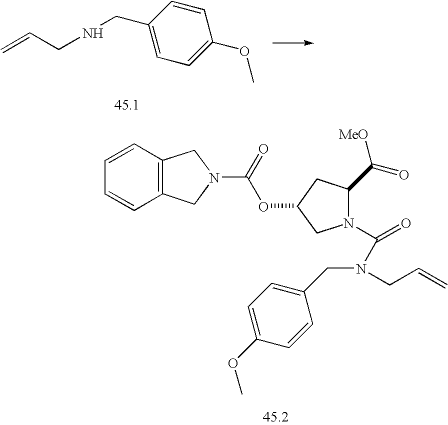

2-methoxycarbonyl-4-(1,3-dihydroisoindolinyl-2-carboxy)pyrrolidine hydrochloride (4.113 g, 16.8 mmol) and DMAP (3.072 g, 25 mmol) in 15 mL dichloromethane was added into triphosgene (1.994 g, 6.7 mmol) in 15 mL dichloromethane at 0° C., after the addition was finished, the mixture was stirred at room temperature for 3 h. Then the mixture was recooled to 0° C., DMAP (3.072 g, 25 mmol) in 15 mL dichloromethane and 45.1 (2.97 g, 16.8 mmol) in 15 mL dichloromethane was added in turn, the reaction mixture was stirred at rt overnight. Then 10 ml dichloromethane was introduced into the mixture, the organic layer was washed with 1N HCl (30 mL), saturated NaHCO₃ (30 mL) and brine(30 mL), dried over anhydrous Na₂SO₄, then purified by column chromatography to produce 5.38 g of 45.2. (Yield : 65%)

LC-MS (ESI): [M+H]⁺=494

### Step 3

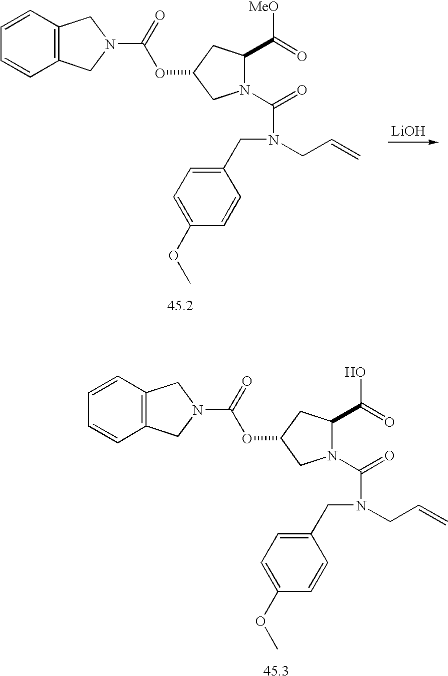

2 mL of LiOH (1N solution) was added to a solution of 45.2 (493 mg, 1 mmol) in dichloromethane (2 mL), the resulting mixture was stirred at room temperature for 2 h. After removal of the solvent under reduced pressure, the residue was used directly to the next step.

LC-MS (ESI): [M+H]⁺=393

### Step 4:

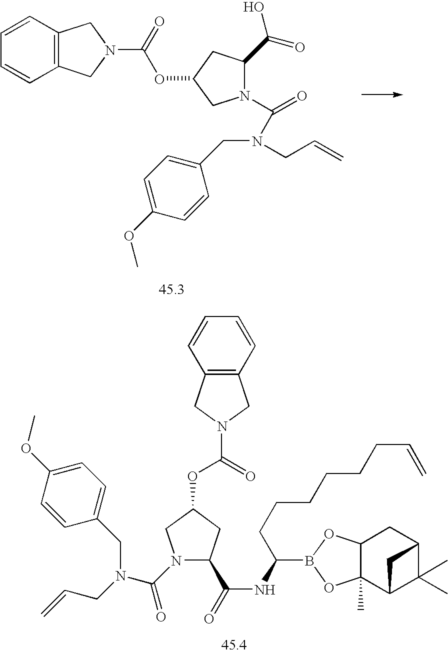

To a suspension solution of 45.3 (2.48 g, 5.18 mmol) and HOBt (1.049 g, 7.76 mmol) in dichloromethane (30 mL) cooled in an ice/water bath was added EDCI (1.488 g, 7.76 mmol). After 3045 minutes, the reaction was cooled down to −15° C.˜−10° C., and (+)-pinanediol (1S)-1-amino-hept-6-ene-1-boronate hydrochloride (3.949 g, 11.1 mmol) was added, then the solution of DIPEA (2.0 g_{;} 3 eq.) in dichloromethane (1:4V) was added over 30 minutes. The reaction mixture was stirred at room temperature for 2.5 hours, then 40 mL dichloromethane was introduced, the organic layer was washed with 1N HCl (30 mL), saturated NaHCO₃ (30 mL) and brine (30 mL), dried over Na₂SO₄. After removal of the solvent, the residue was purified by column chromatography twice (eluted with PE/EA (3:1-2:1)) to yield 2.4 g of 45.4 (Yield: 58%)

LC-MS (ESI): [M+H]⁺=781

### Step 5

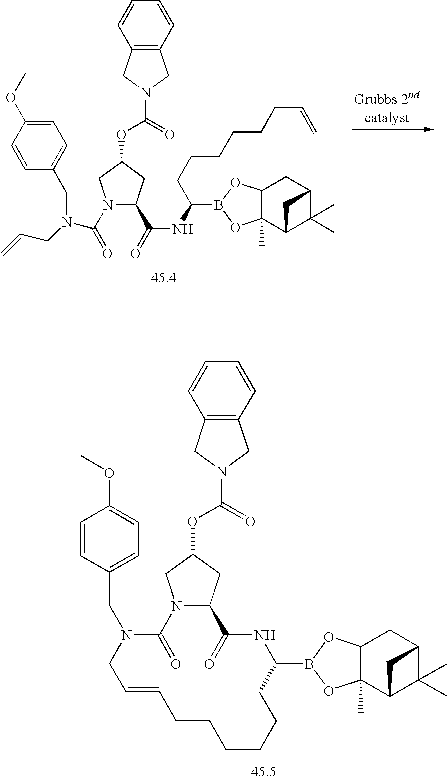

In a three neck round flask with condenser was added 45.4 (592 mg, 0.76 mmol), 1,4-benzoquinone (8.2 mg, 0.1 eqv.) and TFA 1% (mol/mol) in toluene (117 mL, 6.5 mmol/L), the resulting solution was degassed by bubbling argon into the solution at room temperature for 30 min. The flask was then placed in 65° C. oil bath and stirred with argon bubbling into the solution and out of the flask, then Grubbs 2^{nd} catalyst (19.3 mg, 3%) was added in three portions. HPLC indicated that the reaction was completed. The reaction mixture was cooled to 60° C. and 2-mercaptonicotinic acid (18 mg, 0.5 eq.) was added. Toluene was removed by distillation under reduced pressure to around ⅓ of original volume. The organic phase was washed with 0.5 N NaHCO₃ (30 mL), the layers were separated and the organic layer was treated with 18 mg 2-mercaptonicotinic acid and 200 mg activated charcoal at room temperature for 1 hour. The mixture was filtered through Celite, washed with 0.5 N NaHCO₃ (30 mL), 1 N HCl solution (20 mL) and brine (20 mL). Dried with Na₂SO₄, filtered, and then concentrated in vacuo. The resulting residue was applied to a silica gel column and eluted with 5% to 30% ethyl acetate gradient in methylene chloride to get 228 mg of 45.5. (Yield: 40%)

LC-MS (ESI): [M+H]⁺=750

### Step 6

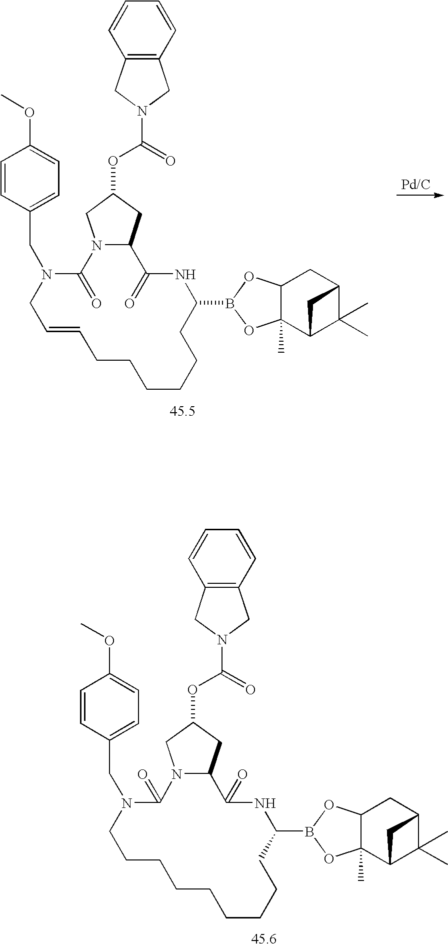

A solution of 45.5 (60 mg, 0.08 mmol) in MeOH (2 mL) was purged with argon for 5 min. Pd/C (12 mg, 20% by weight) was added in one portion and the system was purged with argon for 5 min again. A three way valve with a doubled hydrogen balloon was attached to the flask that was submitted to several cycles of vacuum/H₂. It was finally stirred under hydrogen for 30 min. LCMS showed complete conversion. The catalyst was filtered off through a PTFE filter and the solvent was evaporated under vacuum to get 46 mg of 45.6 as a white solid. (Yield: 75%)

LC-MS (ESI): [M+H]⁺=755

### Step 7

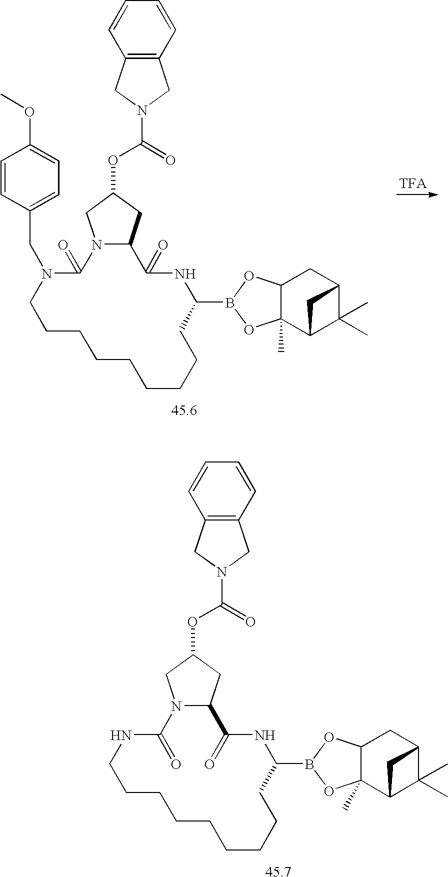

3 mL of TFA was added to a solution of 45.6 (634 mg, 1 mmol) in dichloromethane (3 mL) and the resulting mixture was stirred at room temperature for 2 h. After the removal of the solvent and TFA under reduced pressure the residue was used directly for the next step.

LC-MS (ESI): [M+H]⁺=635

### Step 8

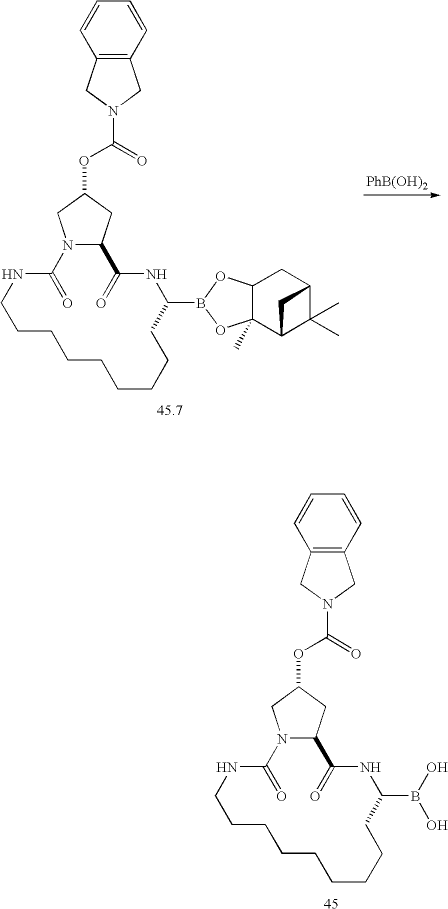

To a solution of 45.7 (171 mg, 0.27 mmol) in MTBE (11 mL, anhydrous/new bottle) and CH₂Cl₂ (1 mL, anhydrous) at room temperature under argon was sequentially added phenyl boronic acid (229 mg, 7 equiv.) and p-toluenesulfonic acid monohydrate (67 mg, 1.3 equiv.). The mixture was stirred for 16-17 hours (overnight under argon) at which time no starting material was detected by LCMS analysis. The solution was then cooled down with an ice/water bath, diluted with MTBE (8 mL) and washed with a precooled 5% NaHCO₃ aqueous solution (10 mL) in the same reaction flask with vigorous stirring. The aqueous layer was separated (with a pipette) and the organic layer washed a second time with 5% NaHCO₃ aqueous solution (10 mL). The organic layer was dried over Na₂SO₄, filtered and the solvents were removed under reduced pressure. The white solid residue was purified by a short silica gel column chromatography using mixtures of MeOH/CH₂Cl₂ as eluents. After purification by column chromatography, 45 was further purified by pre-HPLC to yield 22 mg of 45.

LC-MS (ESI): [M-17]⁺=483

### Example 27

### Compound 46

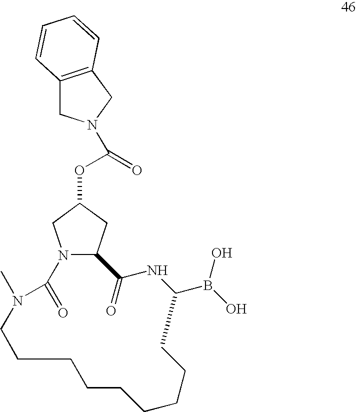

### Step 1

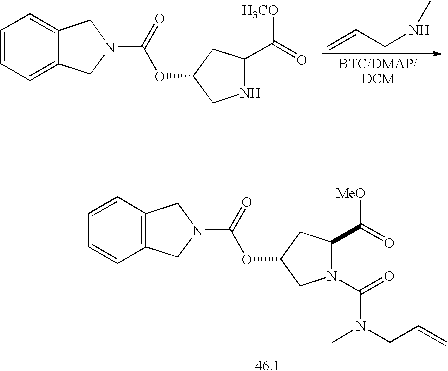

2-methoxycarbonyl-4-(1,3-dihydroisoindolinyl-2-carboxy)pyrrolidine hydrochloride (4.113 g, 16.8 mmol) and DMAP (3.072 g, 25 mmol) in 17 mL dichloromethane was added into triphosgene (1.994 g, 6.7 mmol) in 17 mL dichloromethane at 0° C., after the addition was finished, the mixture was stirred at room temperature for 3 hrs. Then the mixture was recooled to 0° C., DMAP (3.072 g, 25 mmol) in 17 mL dichloromethane and N-methylprop-2-en-1-amine (1.19 g, 16.8 mmol) in 17 mL dichloromethane was added in turn, the reaction mixture was stirred at room temperature overnight. Then 30 mL dichloromethane was introduced into the mixture, the organic layer was washed with 1N HCl (30 mL), saturated NaHCO₃ (30 mL), and brine (30 mL), then dried over anhydrous Na₂SO₄, then purified by column chromatography to yield 3.5 g of 46.1. (Yield: 55%)

LC-MS (ESI): [M+H]⁺=388

### Step 2

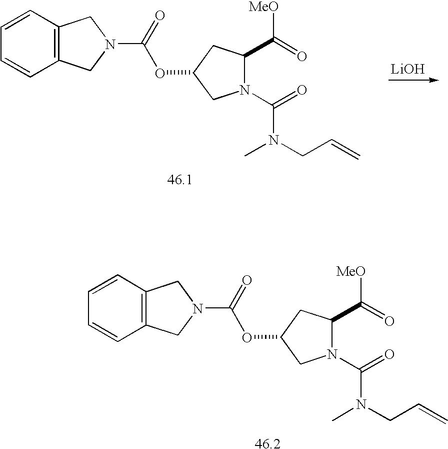

The compound of 46.1 (2.22 g, 5.74 mmol) was dissolved in 6 mL of THF, then 3 mL of aqueous LiOH (1N) was added, the resulting solution was stirred for 4 h at room temperature. Most of the THF was evaporated and the pH was adjusted to 3 with 1N HCl. The solution was extracted with DCM(20 mL×2), the organic phases combined, dried over anhydrous Na₂SO₄, and evaporated to give 46.2 as a solid (2.02 g, 91%).

LC-MS (ESI): [M+H]⁺=374

### Step 3

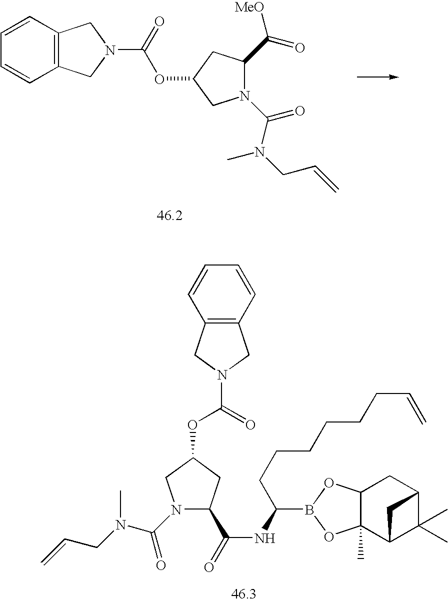

To a suspension solution of 46.2 (2.00 g, 5.18 mmol) and HOBt (1.049 g, 7.76 mmol) in 12 mL of dichloromethane cooled in an ice/water bath was added EDCI (1.488 g, 7.76 mmol). After 30-45 minutes, the reaction was cooled down to −15° C.˜−1° C., (+)-pinanediol (1S)-1-amino-hept-6-ene-1-boronate hydrochloride (3.949 g, 11.1 mmol) was added, then a solution of DIPEA (2.0 g, 3eq.) in dichloromethane (1:4 v/v) was added over 30 minutes. The reaction mixture was stirred at room temperature for 2.5 hours, then 40 mL dichloromethane was introduced, the organic layer was washed with 1N HCl (30 mL), saturated NaHCO₃(30 mL) and brine(30 mL), dried over Na₂SO₄. After the removal of the solvent, the residue was purified by column chromatography twice. (eluted with PE/EA(3:1˜2:1)). 2.26 g of 46.3 was obtained. (Yield: 65%)

LC-MS (ESI): [M+H]⁺=675

### Step 4

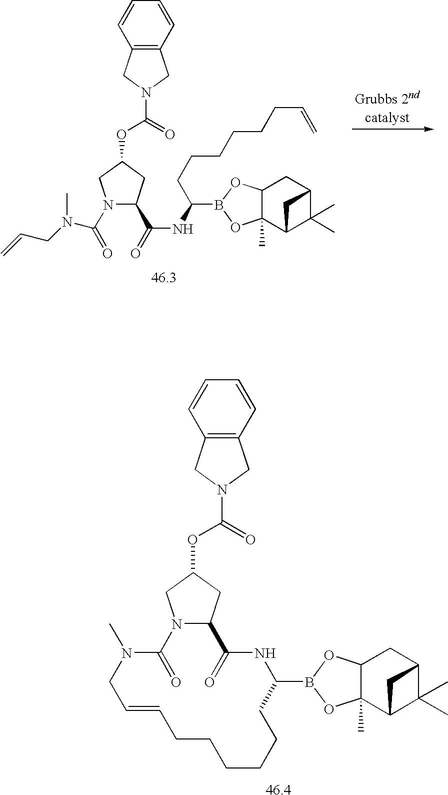

In a three neck round flask with condenser was added 46.3 (512 mg, 0.76 mmol), 1,4-benzoquinone (8.2 mg, 0.1 eq.) and TFA 1% (mol/mol) in toluene (117 mL, 6.5 mmol/L), the resulted solution was degassed by bubbling argon into the solution at room temperature for 30 min. The flask was then placed in 65° C. oil bath and stirred with argon bubbling into the solution and out of the flask, and then Grubbs 2^{nd} catalyst (19.3 mg, 3%) was added in three portions. HPLC indicated that the reaction was completed. The reaction mixture was cooled to 60° C. and 2-mercaptonicotinic acid (18 mg, 0.5 eq.). Toluene was removed by distillation under reduced pressure to around ⅓ of original volume. The organic phase was washed with 0.5 N NaHCO₃ (30 mL), the layers were separated and the organic layer was treated with 18 mg 2-mercaptonicotinic acid and 200 mg activated charcoal at room temperature for 1 hour. The mixture was filtered through Celite, washed with 0.5 N NaHCO₃ (30 mL), 1 N HCl solution (20 mL), and brine(20 mL). After drying with Na₂SO₄, the solution was filtered and concentrated in vacuo. The resulting residue was applied to silica gel and eluted with 5% to 30% ethyl acetate gradient in methylene chloride to yield 196 mg of 46.4. (Yield: 40%)

LC-MS (ESI): [M+H]⁺=647

### Step 5

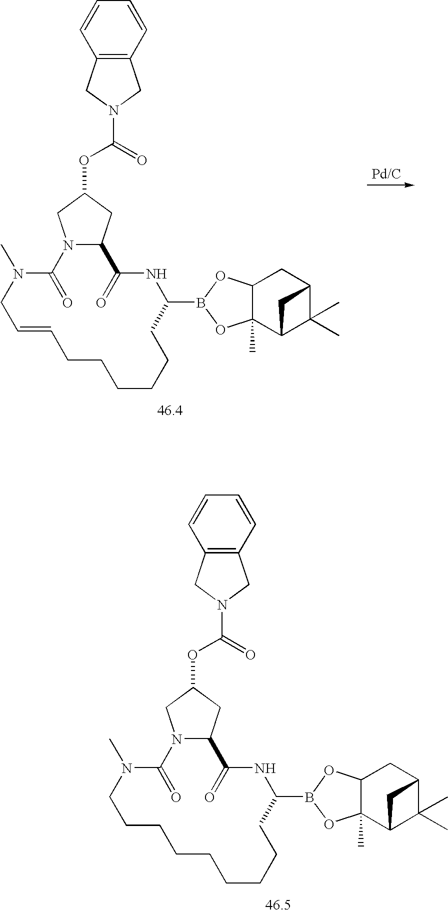

A solution of olefin 46.4 (51 mg, 0.08 mmol) in MeOH (2 mL) was purged with argon for 5 min. Pd/C (10 mg, 20% by weight) was added in one portion and the system was purged with argon for 5 min again. A three way valve with a doubled hydrogen balloon was attached to the flask that was submitted to several cycles of vacuum/H₂. It was finally stirred under hydrogen for 30 min. LCMS showed complete conversion. The catalyst was filtered off through a PTFE filter and the solvent was evaporated under vacuum to get 38 mg of 46.5 as a white solid. (Yield: 75%)

LC-MS (ESI): [M+H]⁺=649

### Step 6:

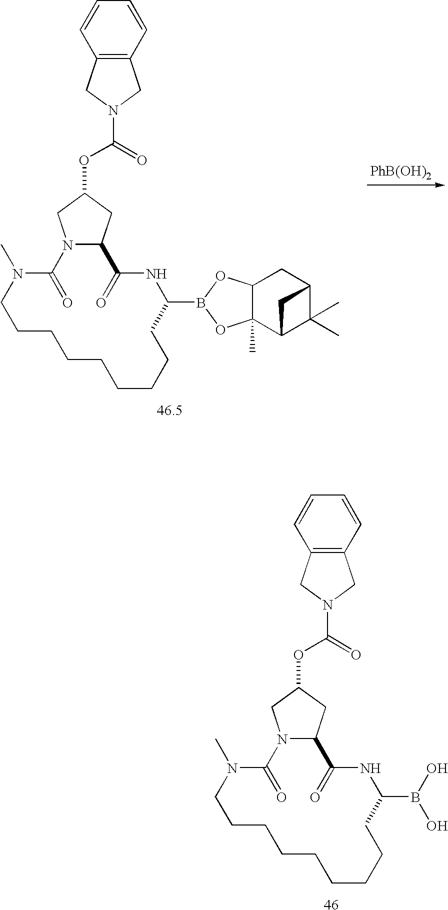

To a solution of the pinanediol ester 46.5 (174 mg, 0.27 mmol) in MTBE (11 mL, anhydrous/new bottle) and CH₂Cl₇ (1 mL, anhydrous) at room temperature under Argon was sequentially added phenyl boronic acid (229 mg, 7 equiv.) and p-toluenesulfonic acid monohydrate (67 mg, 1.3 equiv.). The mixture was stirred for 17 hours (overnight under argon) at which time no starting material was detected by LCMS analysis. The solution was then cooled down with an ice/water bath, diluted with MTBE (8 mL) and washed with a precooled 5% NaHCO₃ aqueous solution (10 mL) in the same reaction flask with vigorous stirring. The aqueous layer was separated and the organic layer washed a second time with 5% NaHCO3 aqueous solution (10 mL). The organic layer was dried over Na₂SO4, filtered (dichloromethane can be used to wash the sodium sulfate) and solvents were removed under reduced pressure. The white solid residue was purified by a short silica gel column chromatography using mixtures of MeOH/CH₂Cl₂ as eluents. The compound was further purified by Pre-HPLC to yield 18 mg of 46.

LC-MS (ESI): [M-17]⁺=497

### Example 28

### Compound 47

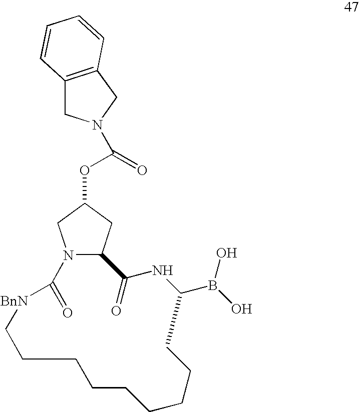

### Step 1

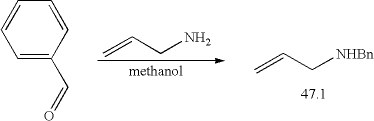

Benzyl aldehyde (10.6 g, 100 mmol), allyl amine (5.7 g, 100 mmol) and methanol (100 mL) were introduced under N₂ atmosphere into a three-necked flask, the resulting solution was stirred at r.t. for 30 min, a few drops of concentrated HCl was introduced, then NaBH₄ (10.8 g, 300 mmol) in methanol (20 mL), the resulting solution was stirred at rt overnight. After completion the reaction, the methanol was evaporated under vacuum, 100 mL water was added, EtOAc was used to extract, the organic layers were combined and dried over Na2SO4, the solvent was removed, and the residue was chromatographed with DCM/Methanol (50:1) to give 6.6 g of 47.1 as a yellowish oil. (Yield: 45%).

### Step 2

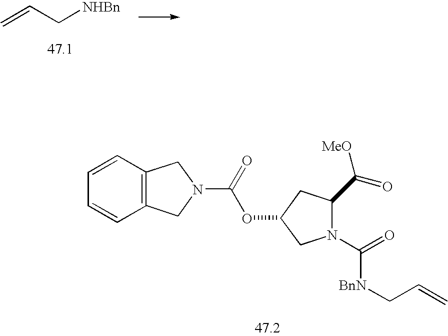

HATU (1.57 g, 4.14 mmol) was added to a solution of 2-methoxycarbonyl-4-(1,3-dihydroisoindolinyl-2-carboxy)pyrrolidine hydrochloride (890 mg, 4.14 mmol) in 10 mL of DCM, then 47.1 (406 mg, 2.76 mmol) in 3 mL of DCM and DIPEA (3.56 g, 27.6 mmol) was added, the mixture was stirred at room temperature for 2 h. The reaction mixture was concentrated to dryness, the residue was diluted with 50 mL of ethyl acetate, washed with 1 N HCl (20 mL), saturated NaHCO₃(20 mL) and brine(20 mL), dried over anhydrous Na₂SO₄, filtered and concentrated under vacuum and purified by chromatography to give 47.2 as solid (1.34 g, 70%).

LC-MS (ESI): [M+H]⁺=450

### Step 3

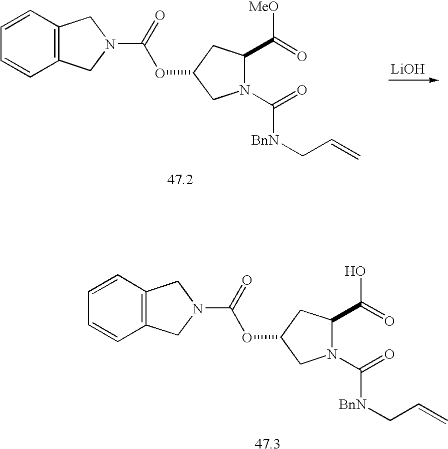

The compound of 47.2 (2.66 g, 5.74 mmol) was dissolved in 5 mL THF.

To this solution was added 5 mL of aqueous LiOH (1N), stirred for 4 h at room temperature. Evaporated most of THF and adjusted the pH to 3 with 1N HCl and extracted with DCM(20 mL×3), combined the organic phase, dried over anhydrous Na₂SO₄, and evaporated the solvent to give 47.3 as solid (2.34 g, 91%).

LC-MS (ESI): [M+H]⁺=450

### Step 4

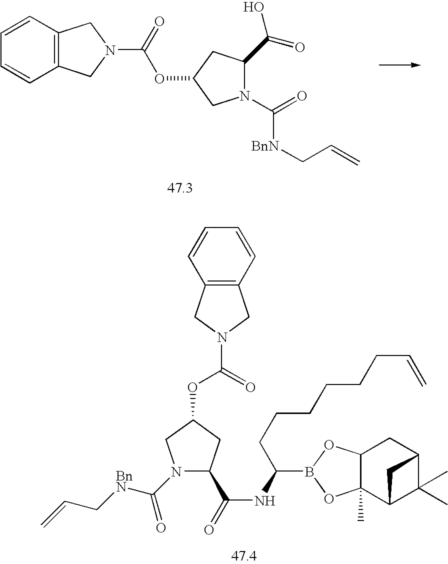

To a suspension of 47.3 (2.32 g, 5.18 mmol) and HOBt (1.049 g, 7.76 mmol) in 20 mL dichloromethane was added EDCI (1.488 g, 7.76 mmol) at 0° C. After 30-45 minutes, the reaction was cooled down to −15° C. to −10° C., (+)-pinanediol (1S)-1-amino-hept-6-ene-1-boronate hydrochloride (3.949 g, 11.1 mmol) was added, then a solution of DIPEA (2.0 g, 3eq.) in dichloromethane (1:4V) was added over 30 minutes. The reaction mixture was stirred at room temperature for 2.5 hours, then 40 mL dichloromethane was added the organic layer was washed with 1N HCl (20 mL), saturated NaHCO₃(20 mL) and brine(20 mL), dried over Na₂SO₄. After the removal of the solvent, the residue was purified by column chromatography (eluted with PE/EA(3:1˜2:1)) to give 233 g of 47.4.(Yield: 61%)

LC-MS (ESI): [M+H]⁺=751

### Step 5

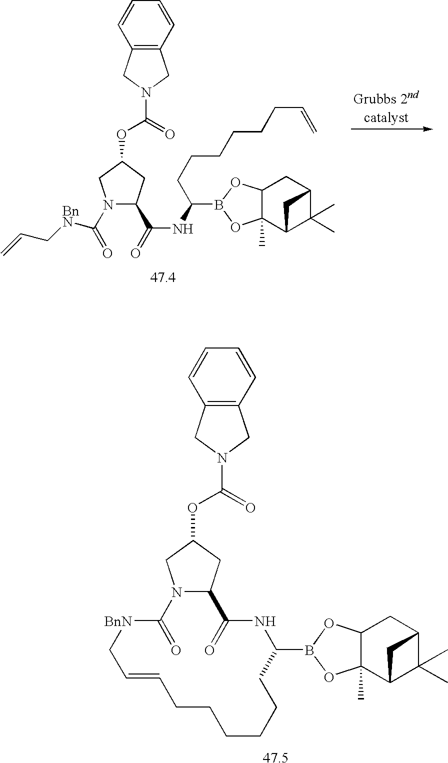

To a three neck round flask with condenser was added 47.4 (570 mg, 0.76 mmol), 1,4-benzoquinone (8.2 mg, 0.1 eqv.) and TFA 1% (mol/mol) in toluene (117 mL, 6.5 mmol/L), the resulted solution was degassed by bubbling argon into the solution at room temperature for 30 min. The flask was then placed in 65° C. oil bath and stirred with argon bubbling into the solution and out of the flask, then Grubbs 2^{nd} catalyst (19.3 mg, 3%) was added in three portions.

HPLC indicated that the reaction was completed. The reaction mixture was cooled to 60° C. then 2-mercaptonicotinic acid (18 mg, 0.5 eq.) was added. Toluene was removed by distillation under reduced pressure to around ⅓ of original volume. The organic phase was washed with 0.5 N NaHCO₃ (30 mL), the layers were separated and the organic layer was treated with 18 mg 2-mercaptonicotinic acid and 200 mg activated charcoal at room temperature for 1 hour. The mixture was filtered through Celite, washed with 0.5 N NaHCO₃ (30 mL), 1 N HCl solution (20 mL), and brine (20 mL). After drying with Na₂SO₄ and filtration, the filtrate was concentrated in vacuo. The resulting residue was applied to silica gel and eluted with 5% to 30% ethyl acetate gradient in methylene chloride to yield 270 mg of 47.5. (Yield: 48%)

LC-MS (ESI): [M+H]⁺=723

### Step 6:

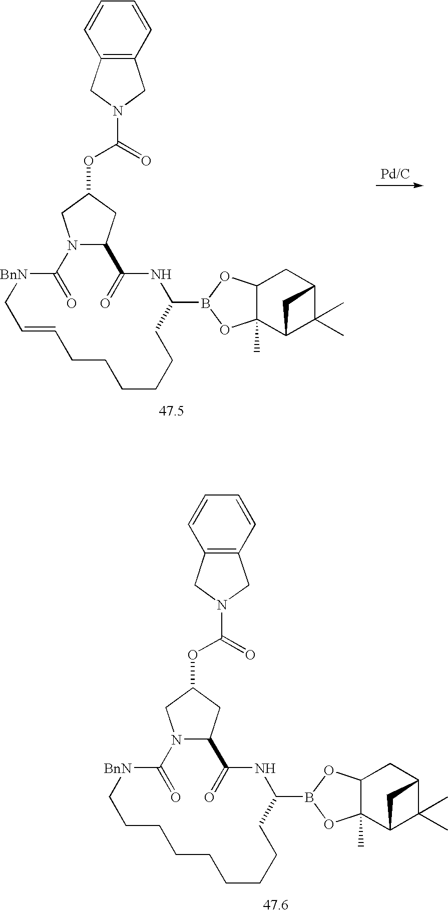

A solution of olefin 47.5 (57 mg, 0.08 mmol) in MeOH (2 mL) was purged with argon for 5 min. Pd/C (12 mg, 20% by weight) was added in one portion and the system was purged with argon for 5 min again. A three way valve with a doubled hydrogen balloon was attached to the flask that was submitted to several cycles of vacuum/H₂. It was finally stirred under hydrogen for 30 min. LCMS showed complete conversion. The catalyst was filtered off through a PTFE filter and the solvent was evaporated under vacuum yield 42 mg of 47.6 as a white solid. (Yield: 75%)

LC-MS (ESI): [M+H]⁺==725

### Step 7

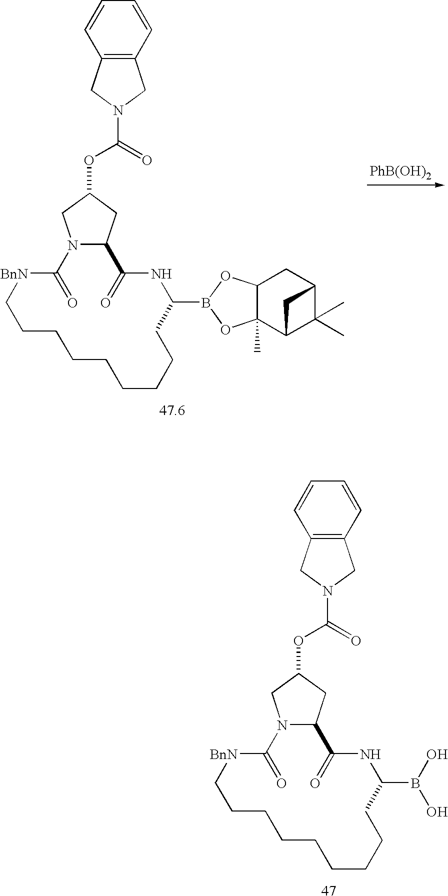

To a solution of 47.6 (195 mg, 0.27 mmol) in MTBE (11 mL, anhydrous/new bottle) and CH₂Cl₂ (1 mL, anhydrous) at room temperature under argon was sequentially added phenyl boronic acid (229 mg, 7 eq.) and p-toluenesulfonic acid monohydrate (67 mg, 1.3 eq.). The mixture was stirred for 24 hours at which time no starting material was detected by LCMS analysis. The solution was then cooled down with an ice/water bath, diluted with MTBE (8 mL) and washed with a precooled 5% NaHCO₃ aqueous solution (10 mL) in the same reaction flask with vigorous stirring. The aqueous layer was separated (with a pipette) and the organic one washed a second time with 5% NaHCO₃ aqueous solution (10 mL). The organic layer was dried over Na₂SO₄, filtered (dichloromethane was used to wash the sodium sulfate) and solvents were removed under reduced pressure. The white solid residue was purified by a short silica gel column chromatography using mixtures of MeOH/CH₂Cl₂ as eluent. 70 mg of 47 was isolated.

LC-MS (ESI): [M-17]⁺=573

1. A compound of Formula (I): 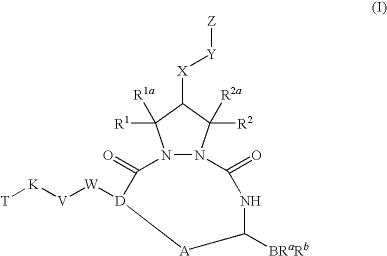
and stereoisomers, solvates, hydrates, tautomers, prodrugs, salts, pharmaceutically acceptable salts, and mixtures thereof, wherein:
R^{a} and R^{b} are independently a hydroxyl or a group that can be hydrolyzed to hydroxyl, or R^{a} and R^{b} together with the boron atom to which they are attached form a cyclic group which can be hydrolyzed to a B(OH)₂ group;
R¹, R^{1a}, R² and R^{2a} are independently H or a substituted or unsubstituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heteroaryl, or heteroarylalkyl group, wherein any carbon atom can be substituted with J;
D is CH₂, CH or N;
when D is CH₂, then W, V, K and T are absent;
when D is CH, then W is C(R⁶)₂, O, S, or NR⁷, and V, K, and T are as defined below;
when D is N then W, V and K are bonds, the bonds taken together forming a single bond, T is as defined below, such that T is bonded directly to D;
wherein R⁶ is independently at each occurrence hydrogen, alkyl, alkenyl, cycloalkyl, cycloalkylalkyl, aryl, aralkyl, heterocyclyl, heterocyclylalkyl, heteroaryl, or heteroarylalkyl, wherein any alkyl, alkenyl, aryl, aralkyl, heterocyclyl, heterocyclylalkyl, heteroaryl or heteroarylalkyl group is substituted with 0-3 J groups;
R⁷ is independently at each occurrence hydrogen, alkyl, cycloalkyl, cycloalkylalkyl, aryl, aralkyl, heterocyclyl, heterocyclylalkyl, heteroaryl, heteroarylalkyl, aralkanoyl, heteroaralkanoyl, C(O)R⁸, SO₂R⁸ or carboxamido, wherein any alkyl, aryl, aralkyl, heterocyclyl, heterocyclylalkyl, heteroaryl, heteroarylalkyl, aralkanoyl, or heteroaralkanoyl is substituted with 0-3 J groups;
R⁸ is alkyl, cycloalkyl, cycloalkylalkyl, aryl, aralkyl, heterocyclyl, heterocyclylalkyl, heteroaryl or heteroarylalkyl;
V is a bond, C(R¹⁰)₂, C(O), S(O), or S(O)₂;
K is a bond, O, S, C(O), S(O), S(O)₂, S(O)(NR¹⁰), or N(R¹⁰);
except when V and K are both bonds, the bonds taken together form a single bond;
R¹⁰ is independently at each occurrence hydrogen, alkyl, cycloalkyl, cycloalkylalkyl, aryl, aralkyl, heterocyclyl, heterocyclylalkyl, heteroaryl or heteroarylalkyl; wherein any R¹⁰ group except hydrogen is substituted with 0-3 J groups;
T is R¹¹, alkyl-R¹¹, alkenyl-R¹¹, alkynyl-R¹¹, OR¹¹, N(R¹¹)₂, C(O)R¹¹, or C(═NOalkyl)-R¹¹;
R¹¹ is independently hydrogen, alkyl, aryl, aralkyl, alkoxy, aryloxy, alkylamino, arylamino, cycloalkyl, cycloalkylidenyl, heterocyclyl, heterocyclylalkyl, heterocyclylalkylidenyl, heteroaryl, or heteroarylalkyl, wherein any R¹¹ group except hydrogen is substituted with 0-3 J groups, or a first R¹¹ and a second R¹¹ together with a nitrogen atom to which they are bound form a mono- or bicyclic ring system substituted with 0-3 J groups;
J is halogen, OR′, OC(O)N(R′)₂, CN, CF₃, OCF₃, R′, O, S, C(O), S(O), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂NHC(O)R′, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein;
each R′ is independently at each occurrence hydrogen, (C₁-C₁₂)-alkyl, (C₃-C₁₀)-cycloalkyl or (C₃-C₁₀)-cycloalkenyl, [(C₃-C₁₀)cycloalkyl or (C₃-C₁₀)-cycloalkenyl]-(C₁-C₁₂)-alkyl, (C₆-C₁₀)-aryl, (C₆-C₁₀)-aryl-(C₁-C₁₂)-alkyl, (C₃-C₁₀)-heterocyclyl, (C₃-C₁₀)-heterocyclyl-(C₁-C₁₂)-alkyl, (C₅-C₁₀)-heteroaryl, or (C₅-C₁₀)-heteroaryl-(C₁-C₁₂)-alkyl, wherein R′ is substituted with 0-3 substituents selected independently from J; or, two R′ groups together with a nitrogen atom to which both R′ groups are attached or with two adjacent nitrogen atoms to which each R′ group is respectively attached form a mono- or bicyclic ring system;
A is a connecting segment comprising a chain of about 6 to about 17 carbon atoms comprising 0 or 1 double bond, wherein any chain carbon atom can bear a C₁-C₆ alkyl group, the chain further comprising 0-2 heteroatoms independently selected from O, S, S(O), S(O)₂, and NR⁷, the chain further comprising 0-3 J groups;
when W is C(R⁶)₂, a bond, or absent:
X is a bond, O, S, C(R⁶)₂ or N(R⁷);
Y is a bond, C(R⁶)₂, C(O), C(O)C(O), S(O), S(O)₂, or S(O)(NR⁷);
except when X and Y are both bonds, the bonds taken together form a single bond;
Z is
a) hydrogen, alkyl, aryl, aralkyl, cycloalkyl, cycloalkylalkyl, heterocyclyl, heterocyclylalkyl, heteroaryl, heteroarylalkyl, OR⁹ , or N(R⁹)₂, wherein any carbon atom is unsubstituted or is substituted with J, and wherein R⁹ is independently at each occurrence hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroarylalkyl, or two R⁹ groups which are bound to a nitrogen atom can form together with the nitrogen atom a 5-11 membered mono- or bicyclic heterocyclic ring system substituted with 0-3 J groups;
b) a substituted aryl or heteroaryl group; wherein any aryl or heteroaryl is substituted with 1-3 J groups;
c) a group of the formula: 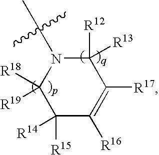
wherein R¹², R¹³, R¹⁴, R¹⁵, R¹⁸, and R¹⁹ are independently H, F, or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or R⁴¹ and R¹⁵ or R¹⁸ and R¹⁹, together with the carbon to which they are attached, can form a C₃₋₆ cycloalkyl group;
R¹⁶ and R¹⁷ are independently H or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹⁶ and R¹⁷ together with the atoms to which they are attached can form a fused substituted or unsubstituted aryl or heteroaryl group;
p is 0 or 1; and
q is 0 or 1;
d) a group of the formula: 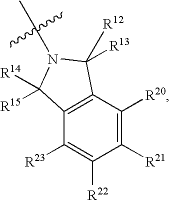
wherein R¹², R¹³, R¹⁴, and R¹⁵ are independently H, F, or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group; or R¹² and R¹³ or R¹⁴ and R¹⁵, together with the carbon to which they are attached, can form a C₃₋₆ cycloalkyl group;
wherein R²⁰, R²¹, R²², R²³ are independently H, F, Cl, Br, I, CN, CF₃, OR²⁴, O—(CH₂)ᵣ—NR²⁵R²⁶, O—(CH₂)ᵣ—OC(O)NR²⁵R²⁶, O—(CH₂)ᵣ—NR²⁵C(O)R²⁶, (CH₂)ᵣ—OR²⁴, OCF₃, NR²⁵R²⁶, (CH₂)ᵣ—NR²⁵R²⁶, SR²⁴, (CH₂)ᵣ—SR²⁴, C(O)R²⁴, C(O)OR²⁴, NR²⁷C(O)R²⁴, C(O)NR²⁵R²⁶, NR²⁷C(O)NR²⁵R²⁶, OC(O)NR²⁵R²⁶, NR²⁷C(O)OR²⁴, NR²⁷SO₂R²⁴, SO₂NR²⁵R²⁶, or a substituted or unsubstituted alkyl, cycloalkyl, cycloalkenyl, cycloalkylalkyl, cycloalkenylalkyl, cycloalkylalkenyl, aryl, aralkyl, aralkenyl, heterocyclyl, heterocyclylalkyl, heterocyclylalkenyl, heteroaryl, heteroarylalkyl, or heteroarylalkenyl group, wherein r is 1, 2, 3, 4, 5, or 6; and
each R²⁴, R²⁵, R²⁶, and R²⁷ is independently H or a substituted or unsubstituted alkyl, cycloalkyl, cycloalkenyl, cycloalkylalkyl, cycloalkenylalkyl, cycloalkylalkenyl, aryl, aralkyl, arylalkenyl, heterocyclyl, heterocyclylalkyl, heterocyclylalkenyl, heteroaryl, heteroarylalkyl, or heteroarylalkenyl group; or, when R²⁵ and R²⁶ are both bound to a nitrogen atom, R²⁵ and R²⁶ together with the nitrogen atom to which they are attached can form a 3-7 membered heterocyclic ring;
e) a group of the formula 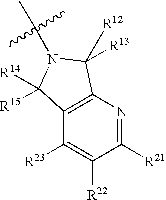
wherein R¹², R¹³, R¹⁴, R¹⁵, R²² and R²³ are as defined above; or
f) a group of the formula 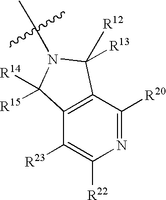
wherein R¹², R¹³, R¹⁴, R¹⁵, R²⁰, R²² and R²³ are as defined above;
wherein a wavy line signifies a point of attachment; or when W is NR⁷, O, or S:
X is O, CH₂, or NH;
Y is C(R⁶)₂ or absent;
Z is a substituted alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl; wherein any alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, alkoxy, aryloxy, alkylthio, arylthio, alkylamino, arylamino, heteroaryl, or heteroarylalkyl is substituted with 1-3 J groups, provided that K and V are both bonds, taken together forming a single bond such that T is bonded directly to W, T is not C(O)R¹¹; or
X is O;
Y is C(O); and
Z is:
aa) a group of the formula 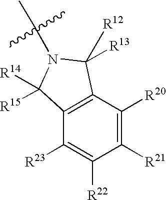
wherein R¹², R¹³, R¹⁴, R¹⁵, R²⁰, R²¹, R²² and R²³ are as defined above; or
bb) a group of the formula 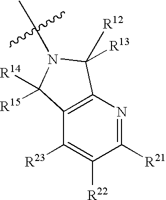
wherein R¹², R¹³, R¹⁴, R¹⁵, R²¹, R²² and R²³ are as defined above; or
cc) a group of the formula 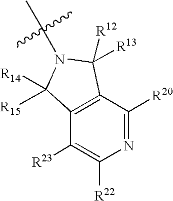
wherein R¹², R¹³, R¹⁴, R¹⁵, R²⁰, R²² and R²³ are as defined above;
wherein a wavy line signifies a point of attachment.

2. The compound of claim 1, wherein X is O.

3. The compound of claim 1, wherein Y is C(O).

4. The compound of claim 1, wherein Y is CH₂.

5. The compound of claim 1, wherein Z is a group of the formula:

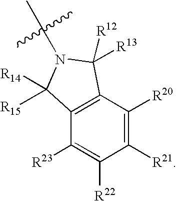

6. The compound of claim 5, wherein R¹², R¹³, R¹⁴, R¹⁵, R²⁰, R²¹, R²² and R²³ are hydrogen.

7. The compound of claim 5 wherein R²⁰ is F.

8. The compound of claim 1, wherein R^{a} and R^{b} are OH or a salt thereof.

9. The compound of claim 1, wherein W is NR⁷.

10. The compound of claim 1, wherein V is C(O).

11. The compound of claim 1, wherein K is O.

12. The compound of claim 1, wherein R¹¹ is alkyl or cycloalkyl.

13. The compound of claim 1, wherein Z is: 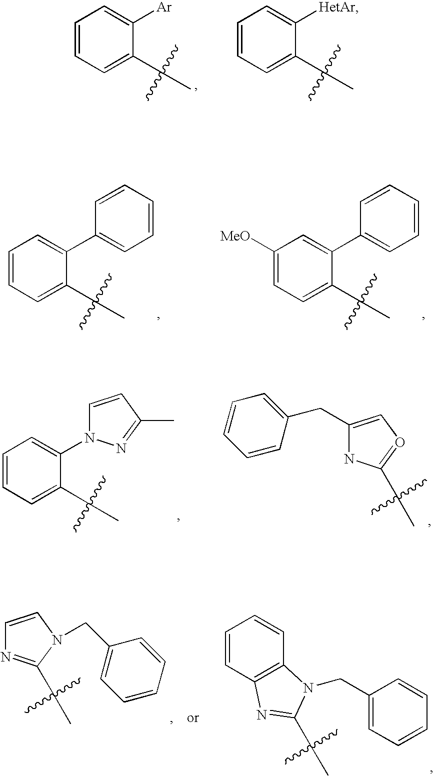
wherein a wavy line signifies a point of attachment, wherein any carbon atom of Z can be substituted with J, wherein Ar is substituted or unsubstituted aryl and HetAr is substituted or unsubstituted hetero aryl.

14. The compound of claim 1 wherein D is CH.

15. The compound of claim 1 wherein D is N.

16. The compound of claim 15 wherein T is H, (C₁-C₆)alkyl, aryl, or aralkyl, wherein any alkyl, aryl, or aralkyl is substituted with 0-3 J groups.

17. The compound of claim 1 wherein D is CH₂.

18. The compound of claim 1 wherein the relative stereochemistry of the compound of Formula (I) is a compound of Formula (IA):

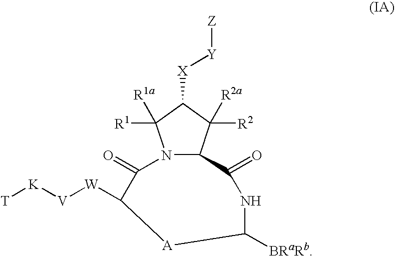

19. The compound of claim 1, wherein R¹¹ is alkyl or cycloalkyl.

20. The compound of claim 1, wherein R¹¹ is t-butyl, neopentyl, or cyclopentyl.

21. The compound of claim 1, comprising:

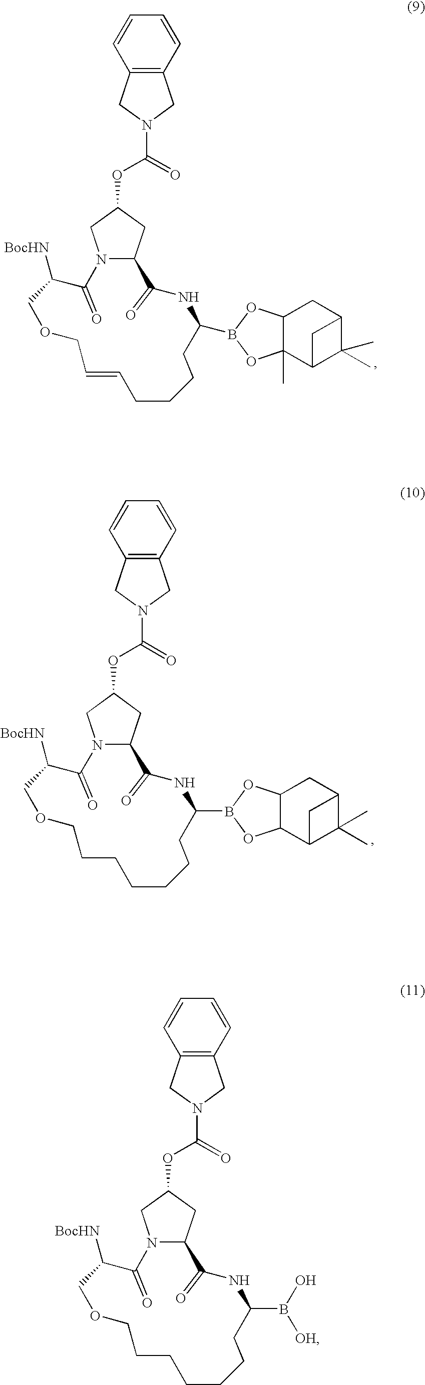

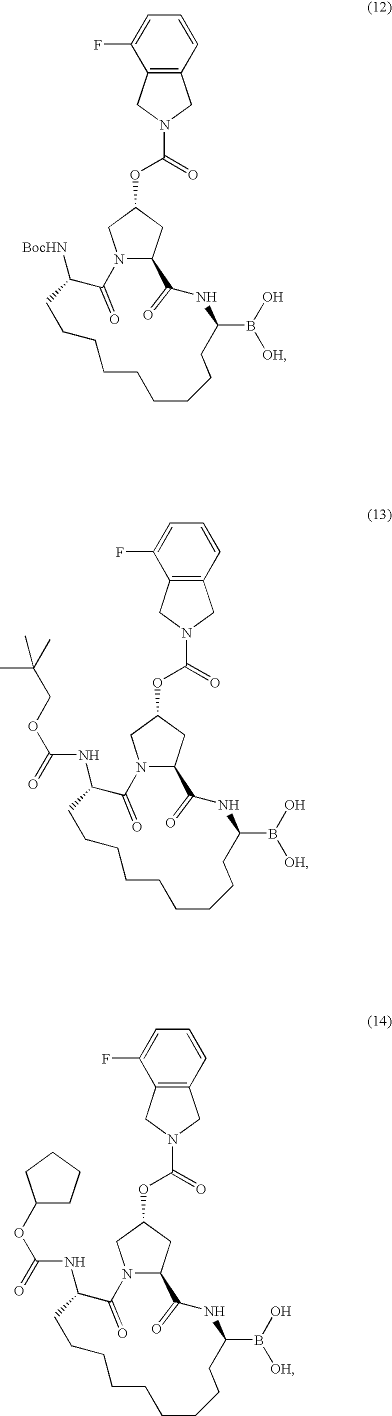

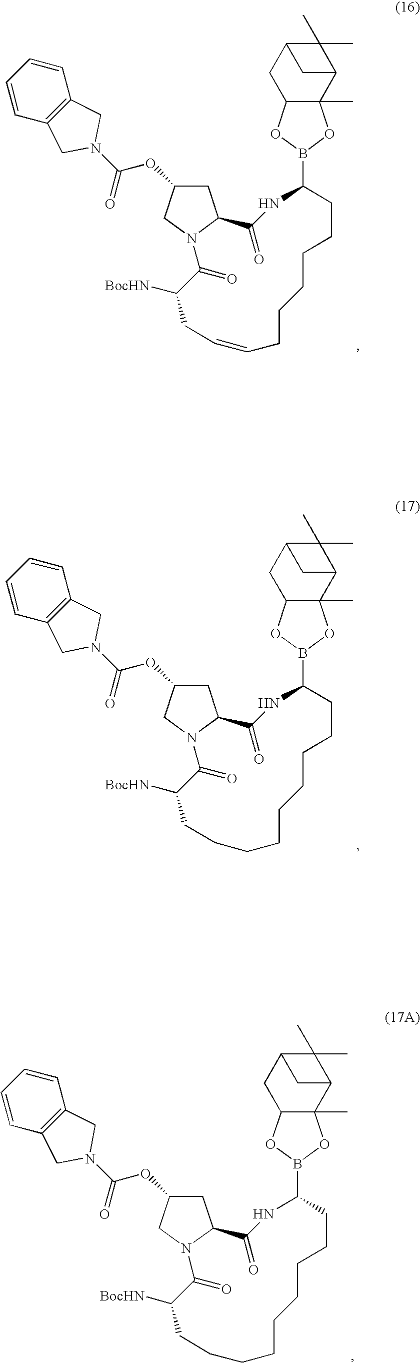

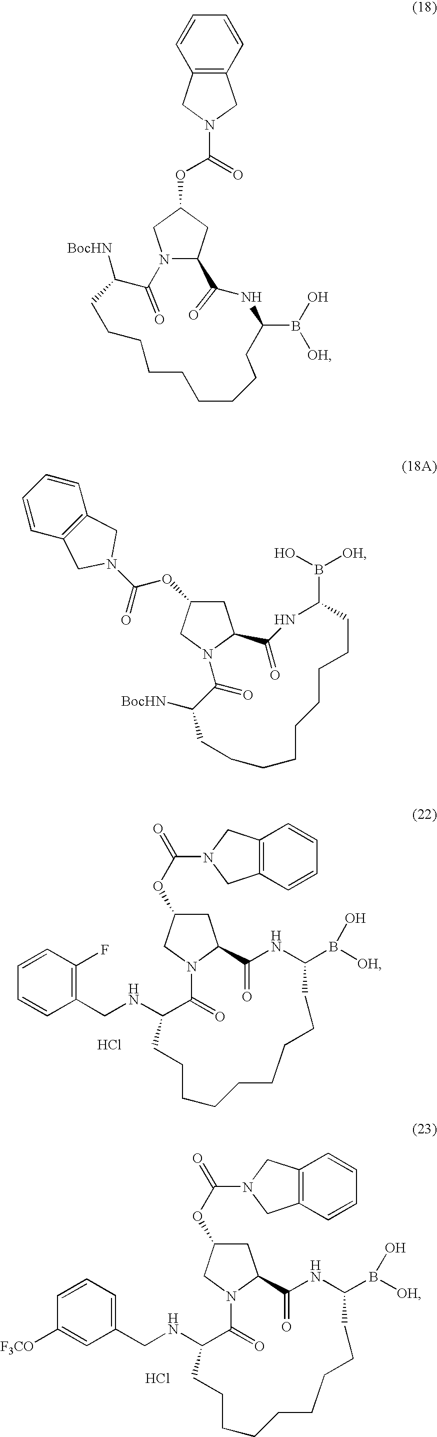

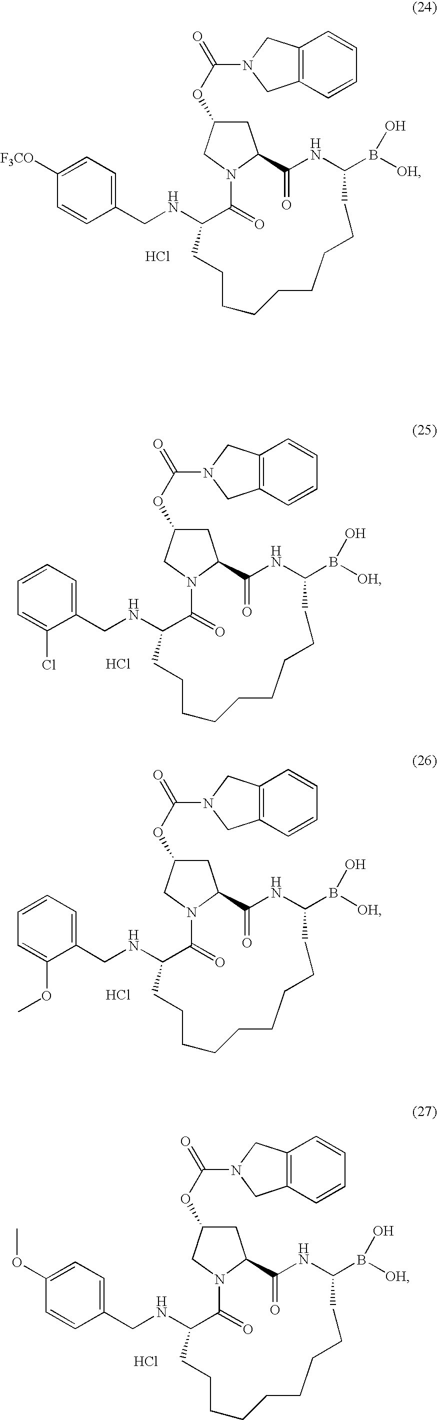

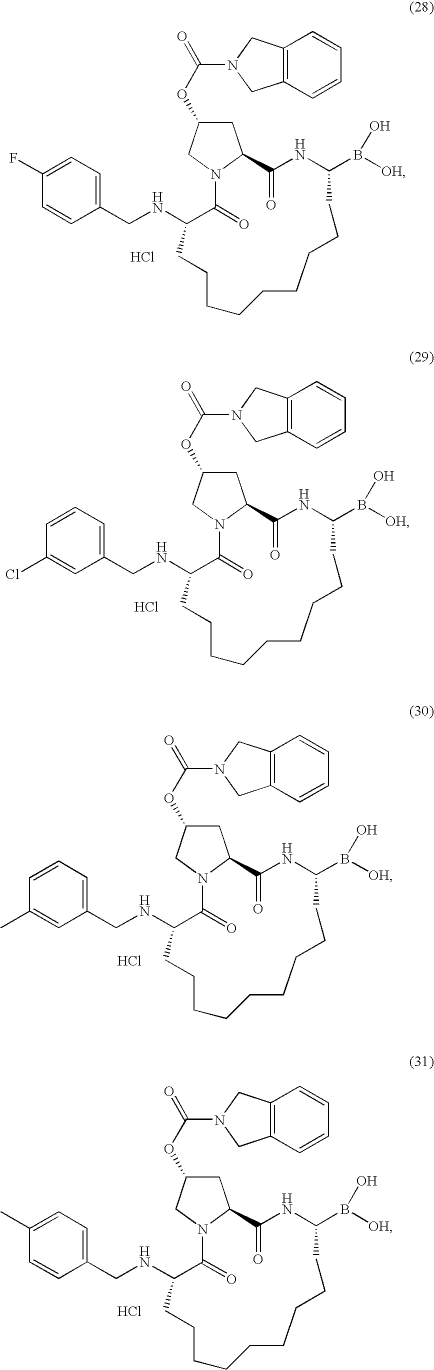

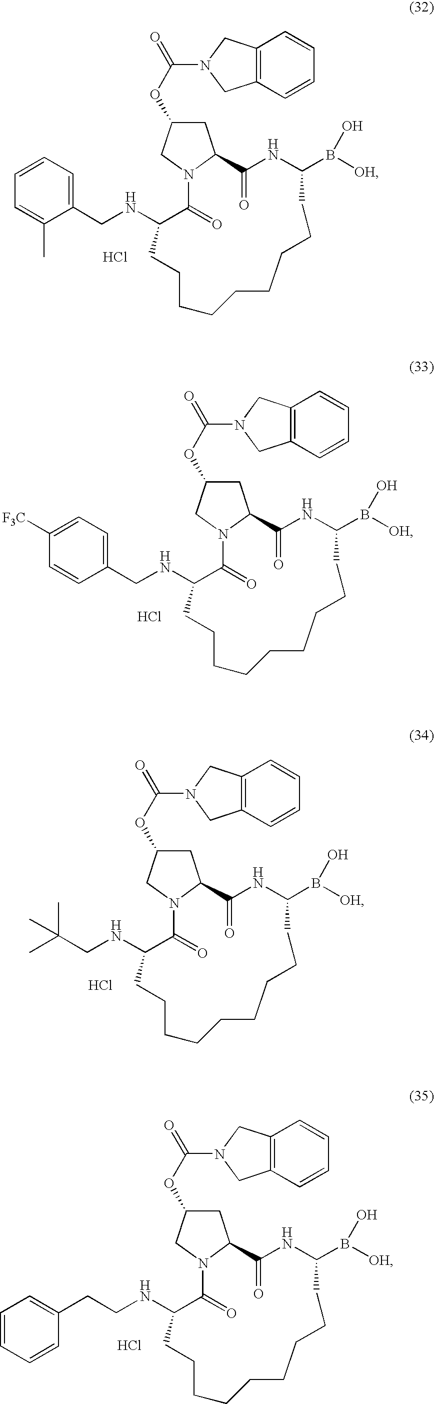

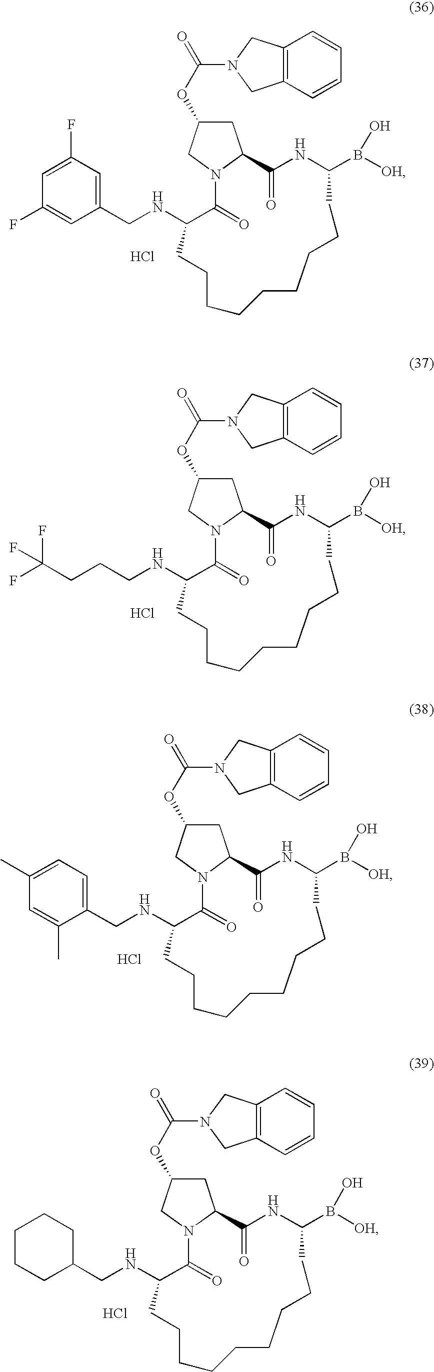

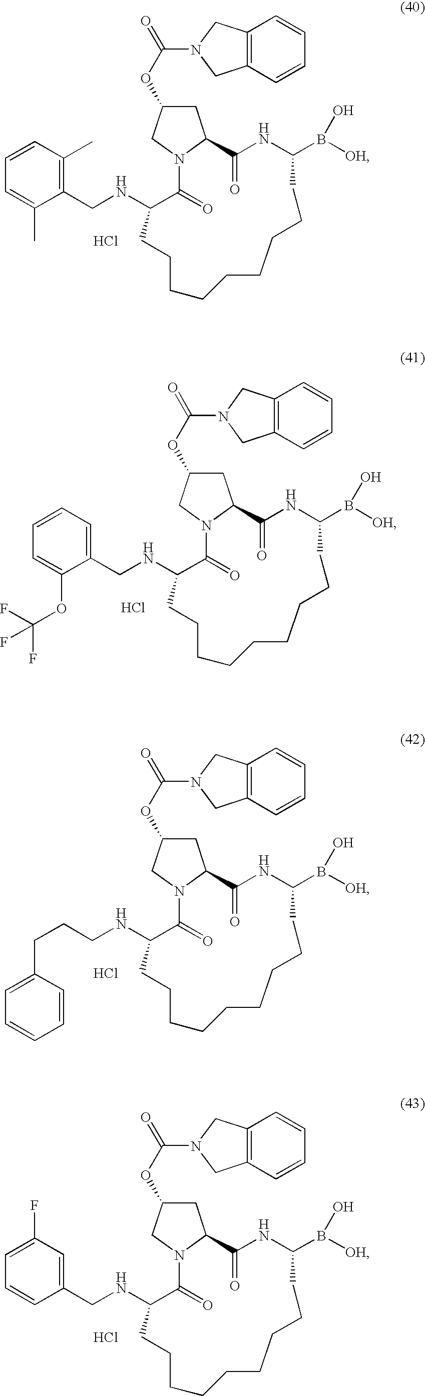

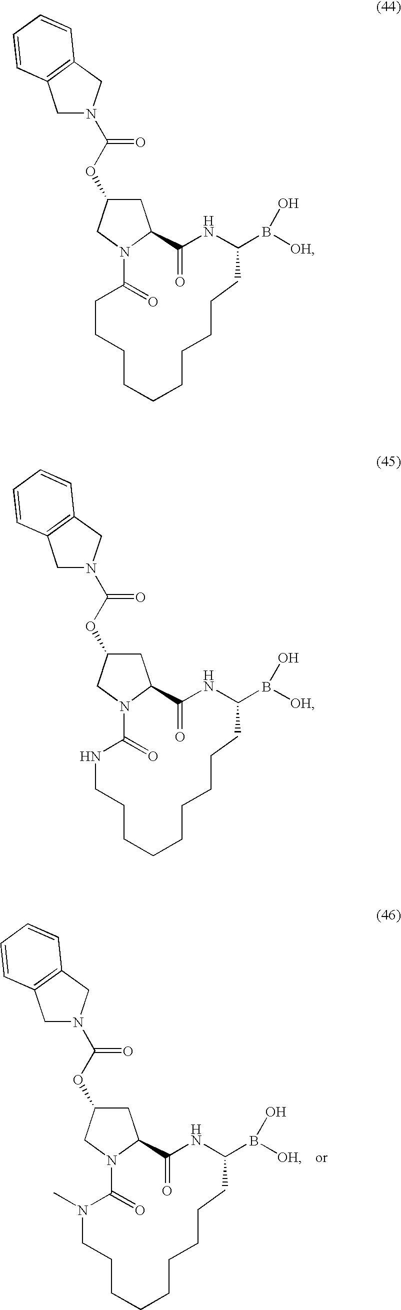

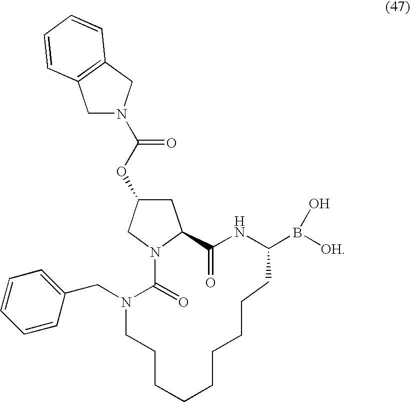

22. A pharmaceutical composition comprising a compound of claim 1 and a suitable excipient.

23. A pharmaceutical combination comprising a compound of claim 1 in a therapeutically effective dose and a second medicament in a therapeutically effective dose.

24. A pharmaceutical composition comprising the combination of claim 23 and a suitable excipient.

25. A method of treatment of a malcondition in a patient in need thereof, wherein inhibition of a hepatitis C viral protease is medically indicated, comprising administering to the patient the compound of claim 1 or the composition of claim 22 in a therapeutically effective amount.

26. A method of treatment of a malcondition in a patient, the malcondition comprising a hepatitis C viral infection, comprising administering to the patient a compound of claim 1 in a therapeutically effective amount.

27. A method of treatment of a malcondition in a patient, the malcondition comprising a hepatitis C viral infection, comprising administering to the patient the pharmaceutical combination of claim 23 or the composition of claim 24 in a therapeutically effective amount.

28. A method of preparing a compound of Formula (I) of claim 1, wherein A comprises a linear alkyl chain comprising a single double bond, comprising contacting a compound of formula (II): 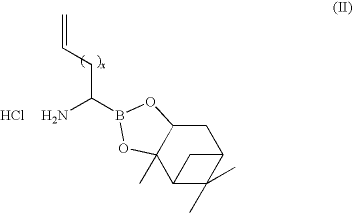
wherein x is 1 to about 7;
and a compound of Formula (III): 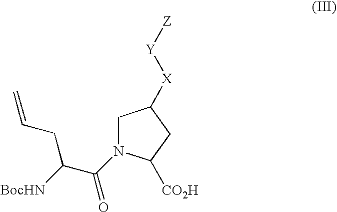
under conditions suitable to bring about formation of an amide bond;
to provide a compound of Formula (IV): 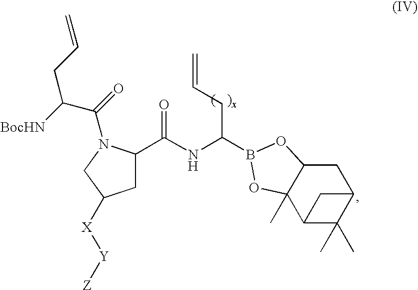
then,
contacting the compound of Formula (IV) with a Ring-Closure-Metathesis (RCM) catalyst to provide a compound of Formula (I) comprising a newly formed ethylenic bond, comprising: 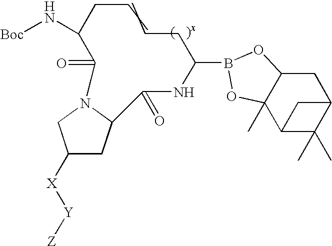
wherein the ethylenic bond can be cis or trans.

29. The method of claim 28 wherein the Ring-Closure-Metathesis catalyst is a Grubbs-Hoveyda 1^{st} generation catalyst.

30. The method of claim 28 further comprising contacting a compound of Formula (I) comprising: 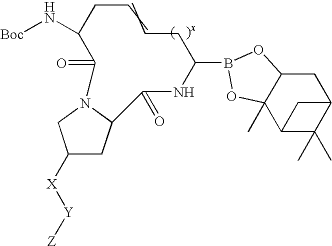
and hydrogen gas in the presence of a hydrogenation catalyst to provide a compound of Formula (I), wherein A comprises a linear alkyl chain with zero double bonds, comprising:

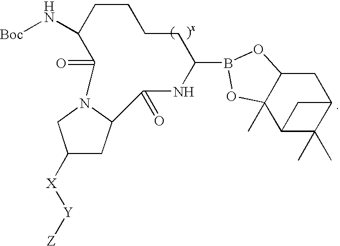

31. The method of claim 28 wherein the compound of Formula (II) is:

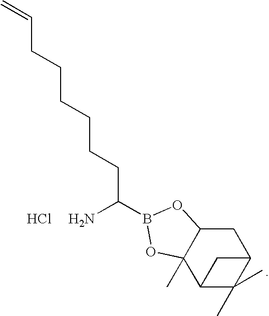

32. A method of preparing a compound of Formula (II) of claim 28: 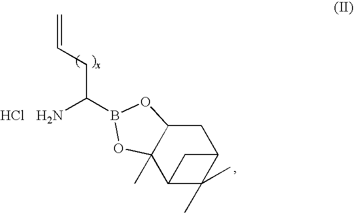
comprising contacting a compound of Formula (V): 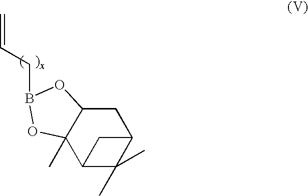
with an carbanion of dichloromethane to provide a compound of Formula (VI): 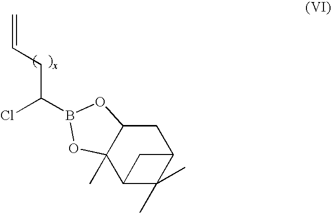
then,
contacting the compound of Formula (VI) and lithium hexamethyldisilazide, followed by aqueous hydrochloric acid, to provide the compound of Formula (II).

33. The method of claim 32 wherein the compound of Formula (V) is:

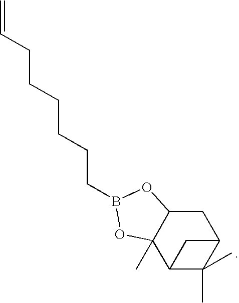

34. The method of claim 32 wherein the compound of Formula (VI) is: [chemical structure]
